(19) United States
(12) Patent Application Publication
(10) Pub. No.: US 2021/0147371 A1
(43) Pub. Date: May 20, 2021

(54) OXADIAZOLINE DERIVATIVES

(71) Applicant: Bayer Aktiengesellschaft, Leverkusen (DE)

(72) Inventors: Andreas GOERTZ, Dormagen (DE); Anne-Sophie REBSTOCK, Champagne au Mont d'or (FR); Sebastien NAUD, Collonges au Mont d'Or (FR); Mathieu GOURGUES, Lyon (FR); Emmanuelle HILT, Dizimieu (FR); Philippe DESBORDES, Lyon (FR); Jeremy DUFOUR, Lyon (FR); Sophie DUCERF, Chasselay (FR); Stephane BRUNET, Saint Andre De Corcy (FR); Vincent THOMAS, Lyon (FR)

(21) Appl. No.: 17/045,943

(22) PCT Filed: Apr. 9, 2019
(86) PCT No.: PCT/EP2019/058891

§ 371 Date: Oct. 7, 2020

(30) Foreign Application Priority Data
Apr. 10, 2018 (EP) 18166500.1

(51) Int. Cl. C07D 271/06; A01N 43/74; C07D 413/04

(52) U.S. Cl. CPC C07D 271/06; C07D 413/04; A01N 43/74

(57) ABSTRACT

The present disclosure relates to oxadiazoline derivatives that may be used as fungicides.

### TECHNICAL FIELD

The present invention relates to oxadiazolines derivatives that may be used as fungicides.

### BACKGROUND

Oxadiazole derivatives are known to be useful as crop protection agents to combat or prevent microorganisms' infestations. For instance, WO2018/029242 discloses oxadiazole derivatives that may be used as fungicides.

Numerous fungicidal agents have been developed until now. However, the need remains for the development of new fungicidal compounds as such, so as to provide compounds being effective against a broad spectrum of fungi, having lower toxicity, higher selectivity, being used at lower dosage rate whilst still allowing effective pest control. It may also be desired to have new compounds to prevent the emergence of fungicides resistances.

The present invention provides new fungicidal compounds which have advantages over known compounds and compositions in at least some of these aspects.

### SUMMARY

The present invention relates to compounds of the formula (I) or salts, N-oxides, solvates thereof:

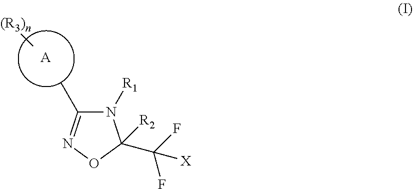

wherein A, R1, R2, R3, X and n are as described herein and uses thereof for controlling unwanted phytopathogenic microorganisms or in methods for controlling unwanted phytopathogenic microorganisms.

The present invention relates to a composition comprising a compound of formula (I) as described herein and one or more agriculturally acceptable carriers.

The present invention also relates to processes for preparing the compounds of formula (I).

### DETAILED DESCRIPTION

### Definitions

The term “halogen” as used herein refers to fluorine, chlorine, bromine or iodine atom.

The term “oxo” as used herein refers to an oxygen atom which is bound to a carbon atom or sulfur atom via a double bound.

The term “C₁-C₆-alkyl” as used herein refers to a saturated, branched or straight hydrocarbon chain having 1, 2, 3, 4, 5 or 6 carbon atoms. Examples of C₁-C₆-alkyl include but are not limited to methyl, ethyl, propyl (n-propyl), 1-methylethyl (iso-propyl), butyl (n-butyl), 1-methylpropyl (sec-butyl), 2-methylpropyl (iso-butyl), 1,1-dimethylethyl (tert-butyl), pentyl, 1-methylbutyl, 2-methylbutyl, 3-methylbutyl, 2,2-dimethylpropyl, 1-ethylpropyl, 1,1-dimethylpropyl, 1,2-dimethylpropyl, hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl. Particularly, said hydrocarbon chain has 1, 2, 3 or 4 carbon atoms (“C₁-C₄-alkyl”), e.g. methyl, ethyl, propyl, iso-propyl, butyl, sec-butyl, iso-butyl or tert-butyl.

The term “C₂-C₆-alkenyl” as used herein refers to an unsaturated, branched or straight hydrocarbon chain having 2, 3, 4, 5 or 6 carbon atoms and comprising at least one double bond. Examples of C₂-C₆-alkenyl include but are not limited to ethenyl (or “vinyl”), prop-2-en-1-yl (or “allyl”), prop-1-en-1-yl, but-3-enyl, but-2-enyl, but-1-enyl, pent-4-enyl, pent-3-enyl, pent-2-enyl, pent-1-enyl, hex-5-enyl, hex-4-enyl, hex-3-enyl, hex-2-enyl, hex-1-enyl, prop-1-en-2-yl (or “isopropenyl”), 2-methylprop-2-enyl, 1-methylprop-2-enyl, 2-methylprop-1-enyl, 1-methylprop-1-enyl, 3-methylbut-3-enyl, 2-methylbut-3-enyl, 1-methylbut-3-enyl, 3-methylbut-2-enyl, 2-methylbut-2-enyl, 1-methylbut-2-enyl, 3-methylbut-1-enyl, 2-methylbut-1-enyl, 1-methylbut-1-enyl, 1,1-dimethylprop-2-enyl, 1-ethylprop-1-enyl, 1-propylvinyl, 1-isopropylvinyl, 4-methylpent-4-enyl, 3-methylpent-4-enyl, 2-methylpent-4-enyl, 1-methylpent-4-enyl, 4-methylpent-3-enyl, 3-methylpent-3-enyl, 2-methylpent-3-enyl, 1-methylpent-3-enyl, 4-methylpent-2-enyl, 3-methylpent-2-enyl, 2-methylpent-2-enyl, 1-methylpent-2-enyl, 4-methylpent-1-enyl, 3-methylpent-1-enyl, 2-methylpent-1-enyl, 1-methylpent-1-enyl, 3-ethylbut-3-enyl, 2-ethylbut-3-enyl, 1-ethylbut-3-enyl, 3-ethylbut-2-enyl, 2-ethylbut-2-enyl, 1-ethylbut-2-enyl, 3-ethylbut-1-enyl, 2-ethylbut-1-enyl, 1-ethylbut-1-enyl, 2-propylprop-2-enyl, 1-propylprop-2-enyl, 2-isopropylprop-2-enyl, 1-isopropylprop-2-enyl, 2-propylprop-1-enyl, 1-propylprop-1-enyl, 2-isopropylprop-1-enyl, 1-isopropylprop-1-enyl, 3,3-dimethylprop-1-enyl, 1-(1,1-dimethylethyl)ethenyl, buta-1,3-dienyl, penta-1,4-dienyl, hexa-1,5-dienyl or methylhexadienyl group.

The term “C₂-C₆-alkynyl” as used herein refers to a branched or straight hydrocarbon chain having 2, 3, 4, 5 or 6 carbon atoms and comprising at least one triple bond. Examples of C₂-C₆-alkynyl include but are not limited to ethynyl, prop-1-ynyl, prop-2-ynyl (or “propargyl”), but-1-ynyl, but-2-ynyl, but-3-ynyl, pent-1-ynyl, pent-2-ynyl, pent-3-ynyl, pent-4-ynyl, hex-1-ynyl, hex-2-ynyl, hex-3-ynyl, hex-4-ynyl, hex-5-ynyl, 1-methylprop-2-ynyl, 2-methylbut-3-ynyl, 1-methylbut-3-ynyl, 1-methylbut-2-ynyl, 3-methylbut-1-ynyl, 1-ethylprop-2-ynyl, 3-methylpent-4-ynyl, 2-methylpent-4-ynyl, 1-methyl-pent-4-ynyl, 2-methylpent-3-ynyl, 1-methylpent-3-ynyl, 4-methylpent-2-ynyl, 1-methyl-pent-2-ynyl, 4-methylpent-1-ynyl, 3-methylpent-1-ynyl, 2-ethylbut-3-ynyl, 1-ethylbut-3-ynyl, 1-ethylbut-2-ynyl, 1-propylprop-2-ynyl, 1-isopropylprop-2-ynyl, 2,2-dimethylbut-3-ynyl, 1,1-dimethylbut-3-ynyl, 1,1-dimethylbut-2-ynyl or 3,3-dimethylbut-1-ynyl group.

The term “C₁-C₆-haloalkyl” as used herein refers to a C₁-C₆-alkyl group as defined above in which one or more hydrogen atoms are replaced with one or more halogen atoms that may be the same or different. Examples of C₁-C₆-haloalkyl include but are not limited to chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl and 1,1,1-trifluoroprop-2-yl.

The term “C₂-C₆-haloalkenyl” as used herein refers to a C₂-C₆-alkenyl group as defined above in which one or more hydrogen atoms are replaced with one or more halogen atoms that may be the same or different.

The term “C₂-C₆-haloalkynyl” as used herein refers to a C₂-C₆-alkynyl group as defined above in which one or more hydrogen atoms are replaced with one or more halogen atoms that may be the same or different.

The term “C₁-C₆-cyanoalkyl” as used herein refers to a C₁-C₆-alkyl group as defined above in which one or more hydrogen atoms are replaced with one or more cyano group.

The term “C₂-C₆-cyanoalkenyl” as used herein refers to a C₂-C₆-alkenyl group as defined above in which one or more hydrogen atoms are replaced with one or more cyano group.

The term “C₂-C₆-cyanoalkynyl” as used herein refers to a C₂-C₆-alkynyl group as defined above in which one or more hydrogen atoms are replaced with one or more cyano group.

The term “C₁-C₆-alkoxy” as used herein refers to a group of formula (C₁-C₆-alkyl)-O—, in which the term “C₁-C₆-alkyl” is as defined herein. Examples of C₁-C₆-alkoxy include but are not limited to methoxy, ethoxy, n-propoxy, 1-methylethoxy, n-butoxy, 1-methylpropoxy, 2-methylpropoxy, 1,1-dimethylethoxy, n-pentoxy, 1-methylbutoxy, 2-methylbutoxy, 3-methylbutoxy, 2,2-dimethylpropoxy, 1-ethylpropoxy, 1,1-dimethylpropoxy, 1,2-dimethylpropoxy, n-hexyloxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy and 1-ethyl-2-methylpropoxy.

The term “C₁-C₆-haloalkoxy” as used herein refers to a C₁-C₆-alkoxy group as defined above in which one or more hydrogen atoms are replaced with one or more halogen atoms that may be the same or different. Examples of C₁-C₆-haloalkoxy include but are not limited to chloromethoxy, bromomethoxy, dichloromethoxy, trichloromethoxy, fluoromethoxy, difluoromethoxy, trifluoromethoxy, chlorofluoromethoxy, dichlorofluoromethoxy, chlorodifluoromethoxy, 1-chloroethoxy, 1-bromoethoxy, 1-fluoroethoxy, 2-fluoroethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, pentafluoroethoxy and 1,1,1-trifluoroprop-2-oxy.

The term “C₁-C₆-hydroxyalkyl” as used herein refers to a C₁-C₆-alkyl group as defined above in which at least one hydrogen atom is replaced with a hydroxyl group. Examples of C₁-C₆-hydroxyalkyl include but are not limited to hydroxymethyl, 1-hydroxyethyl, 2-hydroxyethyl, 1,2-dihydroxyethyl, 3-hydroxypropyl, 2-hydroxypropyl, 1-hydroxypropyl, 1-hydroxypropan-2-yl, 2-hydroxypropan-2-yl, 2,3-dihydroxypropyl and 1,3-dihydroxypropan-2-yl.

The term “C₂-C₆-hydroxyalkenyl” as used herein refers to a C₂-C₆-alkenyl group as defined above in which at least one hydrogen atom is replaced with a hydroxyl group.

The term “C₂-C₆-hydroxyalkynyl” as used herein refers to a C₂-C₆-alkynyl group as defined above in which at least one hydrogen atom is replaced with a hydroxyl group.

The term “C₁-C₆-alkylsulfanyl” as used herein refers to a group of formula (C₁-C₆-alkyl)-S—, in which the term “C₁-C₆-alkyl” is as defined herein. Examples of C₁-C₆-alkylsulfanyl include but are not limited to methylsulfanyl, ethylsulfanyl, propylsulfanyl, isopropylsulfanyl, butylsulfanyl, sec-butylsulfanyl, isobutylsulfanyl, tert-butylsulfanyl, pentylsulfanyl, isopentylsulfanyl, hexylsulfanyl group.

The term “C₁-C₆-haloalkylsulfanyl” as used herein refers to a C₁-C₆-alkylsulfanyl as defined above in which one or more hydrogen atoms are replaced with one or more halogen atoms that may be the same or different. Examples of C₁-C₆-haloalkylsulfanyl include but are not limited to C₁-C₃-haloalkylsulfanyl such as chloromethylsulfanyl, bromomethylsulfanyl, dichloromethylsulfanyl, trichloromethylsulfanyl, fluoromethylsulfanyl, difluoromethylsulfanyl, trifluoromethylsulfanyl, chlorofluoromethylsulfanyl, dichlorofluoromethylsulfanyl, chlorodifluoromethylsulfanyl, 1-chloro-ethylsulfanyl, 1-bromoethylsulfanyl, 1-fluoroethylsulfanyl, 2-fluoroethylsulfanyl, 2,2-difluoroethyl-sulfanyl, 2,2,2-trifluoroethylsulfanyl, 2-chloro-2-fluoroethylsulfanyl, 2-chloro-2,2-difluoroethylsulfanyl, 2,2-dichloro-2-fluoroethylsulfanyl, 2,2,2-trichloroethylsulfanyl, pentafluoroethylsulfanyl and 1,1,1-trifluoroprop-2-ylsulfanyl.

The term “C₁-C₆-alkylsulfonyl” as used herein refers to a group of formula (C₁-C₆-alkyl)-SO₂—, in which the term “C₁-C₆-alkyl” is as defined herein. Examples of C₁-C₆-alkylsulfonyl include but are not limited to methylsulfonyl, ethylsulfonyl, propylsulfonyl, 1-methylethylsulfonyl, butylsulfonyl, 1-methylpropyl-sulfonyl, 2-methylpropylsulfonyl, 1,1-dimethylethylsulfonyl, pentylsulfonyl, 1-methylbutylsulfonyl, 2-methylbutylsulfonyl, 3-methylbutylsulfonyl, 2,2-dimethylpropylsulfonyl, 1-ethylpropylsulfonyl, 1,1-dimethylpropylsulfonyl, 1,2-dimethylpropylsulfonyl, hexylsulfonyl, 1-methylpentylsulfonyl, 2-methyl-pentylsulfonyl, 3-methylpentylsulfonyl, 4-methylpentylsulfonyl, 1,1-dimethylbutylsulfonyl, 1,2-dimethylbutylsulfonyl, 1,3-dimethylbutylsulfonyl, 2,2-dimethylbutylsulfonyl, 2,3-dimethylbutylsulfonyl, 3,3-dimethylbutylsulfonyl, 1-ethylbutylsulfonyl, 2-ethylbutylsulfonyl, 1,1,2-trimethylpropylsulfonyl, 1,2,2-trimethylpropylsulfonyl, 1-ethyl-1-methylpropylsulfonyl and 1-ethyl-2-methylpropylsulfonyl.

The term “C₁-C₆-alkylcarbonyl” as used herein refers to a group of formula (C₁-C₆-alkyl)-C(═O)—, in which the term “C₁-C₆-alkyl” is as defined herein.

The term “C₁-C₆-alkoxycarbonyl” as used herein refers to a group of formula (C₁-C₆-alkoxy)-C(═O)—, in which the term “C₁-C₆-alkoxy” is as defined herein.

The term “C₁-C₆-alkylamino” as used herein refers to an amino radical wherein one of the hydrogen atoms is replaced with a C₁-C₆-alkyl which is as defined herein. Examples of C₁-C₆-alkylamino include but are not limited to N-methylamino, N-ethylamino, N-isopropylamino and N-propylamino.

The term “C₁-C₆-dialkylamino” as used herein refers to an amino radical having two independently selected C₁-C₆-alkyl groups as defined herein. Examples of C₁-C₆-dialkylamino include but are not limited to N,N-dimethylamino, N,N-diethylamino, N,N-diisopropylamino, N-ethyl-N-methylamino, N-methyl-N-n-propylamino, N-isopropyl-N-n-propylamino and N-tert-butyl-N-methylamino.

The term “non-aromatic C₃-C₁₂-carbocycle” or “carbocyclyl” as used herein refers to a non-aromatic, saturated or partially unsaturated, hydrocarbon ring system in which all of the ring members, which vary from 3 to 12, are carbon atoms. The ring system may be monocyclic or polycyclic (fused, spiro or bridged). Non-aromatic C₃-C₁₂-carbocycles include C₃-C₁₂-cycloalkyl (mono or bicyclic), C₃-C₁₂-cycloalkenyl (mono or bicyclic) and bicylic system comprising an aryl (e.g. phenyl) fused to a monocyclic C₃-C₈-cycloalkyl (e.g. tetrahydronapthalenyl, indanyl). The non-aromatic C₃-C₁₂-carbocyle can be attached to the parent molecular moiety through any carbon atom.

The term “C₃-C₁₂-cycloalkyl” as used herein refers to a saturated, monovalent, mono- or bicylic hydrocarbon ring which contains 3, 4, 5, 6, 7, 8, 9, 10, 11 or 12 carbon atoms. Examples of monocyclic C₃-C₈-cycloalkyls include but are not limited to cyclopropyl, cyclobutyl, cyclopentyl, cyclohexyl, cycloheptyl, or cyclooctyl. Examples of bicyclic C₆-C₁₂-cycloalkyls include but are not limited to bicyclo[3.1.1]heptane, bicyclo[2.2.1]heptane, bicyclo[2.2.2]octane, bicyclo[3.2.2]nonane, bicyclo[3.3.1]nonane, bicyclo[4.2.0]octyl, octahydropentalenyl and bicyclo[4.2.1]nonane.

The term “C₃-C₁₂-cycloalkenyl” as used herein refers to a partially unsaturated, monovalent, mono- or bicylic hydrocarbon ring which contains 3, 4, 5, 6, 7, 8, 9, 10, 11 or 12 carbon atoms. Examples of monocyclic C₃-C₈-cycloalkenyl group include but are not limited to cyclobutenyl, cyclopentenyl, cyclohexenyl, cycloheptenyl and cyclooctenyl group. Examples of bicyclic C₆-C₁₂-cycloalkenyl group include but are not limited to bicyclo[2.2.1]hept-2-enyl or bicyclo[2.2.2]oct-2-enyl.

The term “aromatic C₆-C₁₄-carbocycle” or “aryl” as used herein refers to an aromatic hydrocarbon ring system in which all of the ring members, which vary from 6 to 14, preferably from 6 to 10, are carbon atoms. The ring system may be monocyclic or fused polycyclic (e.g. bicyclic or tricyclic). Examples of aryl include but are not limited to phenyl, azulenyl, naphthyl and fluorenyl. The aryl can be attached to the parent molecular moiety through any carbon atom. It is further understood that when said aryl group is substituted with one or more substituents, said substituent(s) may be at any positions on said aryl ring(s). Particularly, in the case of aryl being a phenyl group, said substituent(s) may occupy one or both ortho positions, one or both meta positions, or the para position, or any combination of these positions.

The term “3- to 14-membered non-aromatic heterocycle” or “heterocyclyl” as used herein refers to a saturated or unsaturated non-aromatic ring system comprising 1 to 4 heteroatoms independently selected from the group consisting of oxygen, nitrogen and sulfur. If the ring system contains more than one oxygen atoms, they are not directly adjacent. Non aromatic heterocycles include 3- to 7-membered monocyclic non-aromatic heterocycles and 6- to 14-membered polycyclic (e.g. bicyclic or tricyclic) non-aromatic heterocycles. The 3- to 14-membered non-aromatic heterocycle can be connected to the parent molecular moiety through any carbon atom or nitrogen atom contained within the heterocycle.

The term “3- to 7-membered monocyclic non-aromatic heterocycle” as used herein refers to a 3-, 4-, 5-, 6- or 7-membered monocyclic ring system containing 1, 2 or 3 heteroatoms independently selected from the group consisting of oxygen, nitrogen and sulfur where the ring system is saturated or unsaturated but not aromatic. For instance, the heterocycle may comprise one to three nitrogen atoms, or one or two oxygen atoms, or one or two sulfur atoms, or one to three nitrogen atoms and one oxygen atom, or one to three nitrogen atoms and a sulfur atom or one sulfur atom and one oxygen atom. Examples of saturated non-aromatic heterocycles include but are not limited to 3-membered ring such as oxiranyl, aziridinyl, 4-membered ring such as azetidinyl, oxetanyl, thietanyl, 5-membered ring such as tetrahydrofuranyl, 1,3-dioxolanyl, tetrahydrothienyl, pyrrolidinyl, pyrazolidinyl, imidazolidinyl, triazolidinyl, isoxazolidinyl, oxazolidinyl, oxadiazolidinyl, thiazolidinyl, isothiazolidinyl, thiadiazolidinyl, 6-membered ring such as piperidinyl, hexahydropyridazinyl, hexahydropyrimidinyl, piperazinyl, triazinanyl, hexahydrotriazinyl, tetrahydropyranyl, dioxanyl, tetrahydrothiopyranyl, dithianyl, morpholinyl, 1,2-oxazinanyl, oxathianyl, thiomorpholinyl or 7-membered ring such as oxepanyl, azepanyl, 1,4-diazepanyl and 1,4-oxazepanyl. Examples of unsaturated non-aromatic hererocyles include but are not limited to 5-membered ring such as dihydrofuranyl, 1,3-dioxolyl, dihydrothienyl, pyrrolinyl, dihydroimidazolyl, dihydropyrazolyl, isoxazolinyl, dihydrooxazolyl, dihydrothiazolyl or 6-membered ring such as pyranyl, thiopyranyl, thiazinyl and thiadiazinyl.

The term “6- to 14-membered polycyclic non-aromatic heterocycle” as used herein refers to a 6-, 7-, 8-, 9-, 10-, 11-, 12-, 13- or 14-membered polycyclic (e.g. bicyclic or tricyclic) ring system containing 1, 2 or 3 heteroatoms independently selected from the group consisting of oxygen, nitrogen and sulfur where the ring system is saturated or unsaturated but not aromatic. Non-aromatic bicyclic heterocycles may consist of a monocyclic heteroaryl as defined herein fused to a monocyclic C₃-C₈-cycloalkyl, a monocyclic C₃-C₈-cycloalkenyl or a monocyclic non-aromatic heterocycle or may consist of a monocyclic non-aromatic heterocycle fused either to a phenyl, a monocyclic C₃-C₈-cycloalkyl, a monocyclic C₃-C₈-cycloalkenyl, a monocyclic non-aromatic heterocycle or a monocyclic heteroaryl. Examples of bicyclic non-aromatic heterocycle include but are not limited to 9-membered ring such as indolinyl, isoindolinyl, dihydrobenzofuranyl, tetrahydrobenzothienyl 1,3-benzodioxolyl or 10-membered ring such as dihydroquinolinyl, dihydroisoquinolinyl, tetrahydroquinolinyl, tetrahydroisoquinolinyl, 2,3-dihydro-1,4-benzodioxinyl, chromanyl and chromenyl. An example of tricyclic non-aromatic heterocycle includes but is not limited to 14-membered ring such as xanthenyl.

The term “5- to 14-membered aromatic heterocycle” or “heteroaryl” as used herein refers to an aromatic ring system comprising 1 to 4 heteroatoms independently selected from the group consisting of oxygen, nitrogen and sulfur. If the ring system contains more than one oxygen atom, they are not directly adjacent. Aromatic heterocycles include 5- or 6-membered monocyclic aromatic heterocycles and 6- to 14-membered polycyclic (e.g. bicyclic or tricyclic) aromatic heterocycles. The 5- to 14-membered aromatic heterocycle can be connected to the parent molecular moiety through any carbon atom or nitrogen atom contained within the heterocycle.

The term “5- or 6-membered monocyclic aromatic heterocycle” or “monocyclic heteroaryl” as used herein refers to a 5- or 6-membered monocyclic ring system containing 1, 2, 3 or 4 heteroatoms independently selected from the group consisting of oxygen, nitrogen and sulfur. Examples of 5-membered monocyclic heteroaryl include but are not limited to furyl (furanyl), thienyl, pyrrolyl, pyrazolyl, imidazolyl, triazolyl, tetrazolyl, isoxazolyl, oxazolyl, oxadiazolyl, oxatriazolyl, isothiazolyl, thiazolyl, thiadiazolyl and thiatriazolyl. Examples of 6-membered monocyclic heteroaryl include but are not limited to pyridinyl, pyridazinyl, pyrimidinyl, pyrazinyl, triazinyl, tetrazinyl.

The term “6- to 14-membered polycyclic aromatic heterocycle” or “polycyclic heteroaryl” as used herein refers to a 6-, 7-, 8-, 9-, 10-, 11-,12-, 13- or 14-membered polycyclic (e.g. bicyclic or tricyclic) ring system containing 1, 2 or 3 heteroatoms independently selected from the group consisting of oxygen, nitrogen and sulfur. Aromatic bicyclic heterocycles may consist of a monocyclic heteroaryl as defined herein fused to a phenyl or to a monocyclic heteroaryl. Examples of bicyclic aromatic heterocycle include but are not limited to 9-membered ring such as indolyl, indolizinyl, isoindolyl, benzimadozolyl, imidazopyridinyl, indazolyl, benzotriazolyl, purinyl, benzofuranyl, benzothiophenyl, benzothiazolyl, benzoxazolyl and benzisoxazolyl or 10-membered ring such as quinolinyl, isoquinolinyl, cinnolinyl, quinazolinyl, quinoxalinyl, phthalazinyl, naphthyridinyl, pteridinal and benzodioxinyl. Examples of tricyclic aromatic heterocyle include but are not limited to carbazolyl, acridinyl and phenazinyl.

As used herein, when a group is said to be “substituted”, the group may be substituted with one or more substituents. The expression “one or more substituents” refers to a number of substituents that ranges from one to the maximum number of substituents possible based on the number of available bonding sites, provided that the conditions of stability and chemical feasibility are met.

The term “leaving group” as used herein is to be understood as meaning a group which is displaced from a compound in a substitution or an elimination reaction, for example a halogen atom, a trifluoromethanesulphonate (“triflate”) group, alkoxy, methanesulphonate, p-toluenesulphonate, etc.

The term “C₁-C₆-(halo)alkyl” as used herein in the definition of composite groups (e.g. —C₁-C₆-(halo)alkyl-NR^{b}C(═S)N(R^{b})₂) is to be understood as meaning that the C₁-C₆-alkyl group is optionally substituted by one or more halogen atoms that may be the same or different. In other words, the term halo into brackets designates the optional presence of one or more halogen substitutents.

The expression “wherein any carbocyclyl, heterocyclyl, aryl and heteroaryl group may be substituted by one or more substituents that may be the same or different” indicates that any carbocyclyl, heterocyclyl, aryl and heteroaryl group per se or any carbocyclyl, heterocyclyl, aryl and heteroaryl group as moieties of a given substituent (e.g. —C₁-C₆-alkyl-heteroaryl; —O-aryl) may be substituted by one or more substituents that may be the same or different.

The present invention relates to compounds of the formula (I):

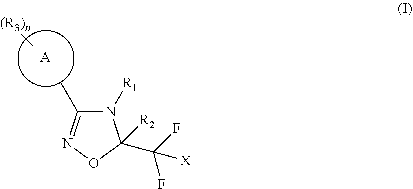

wherein:
A is selected from the group consisting of carbocyclyl, heterocyclyl, aryl and heteroaryl group;
R1 is selected from the group consisting of hydrogen, C₁-C₆-alkyl, C₂-C₆-alkenyl, C₂-C₆-haloalkenyl, C₂-C₆-alkynyl, C₂-C₆-haloalkynyl, C₃-C₃-cycloalkyl, —C(═O)R^{a}, —C(═O)OR^{a} and —C(═O)N(R^{a})₂,
   wherein said C₁-C₆-alkyl may be substituted by one or more substituents independently selected from the group consisting of halogen, hydroxy, cyano, mercapto, sulfinyl, sulfonyl, nitro, amino, C₁-C₆-alkoxy, C₁-C₆-haloalkoxy, C₁-C₆-alkylsulfanyl, C₁-C₆-alkylsulfinyl, C₁-C₆-alkylsulfonyl, —C(═O)—C₁-C₆-alkyl, —C(═O)—C₁-C₆-alkoxy, —C═O—C₁-C₆-alkylamino, —C═O—C₁-C₆-dialkylamino, carbocyclyl, heterocyclyl, aryl and heteroaryl,
      wherein said carbocyclyl, heterocyclyl, aryl and heteroaryl may be substituted by one or more substituents independently selected from the group consisting of halogen, hydroxy, oxo, cyano, C₁-C₆-alkyl, C₁-C₆-haloalkyl, C₁-C₆-alkoxy and C₁-C₆-haloalkoxy,
   wherein said C₃—C-cycloalkyl may be substituted by one or more substituents independently selected from the group consisting of halogen, hydroxy, oxo, cyano, C₁-C₆-alkyl, C₁-C₆-haloalkyl, C₁-C₆-alkoxy and C₁-C₆-haloalkoxy,
   wherein R^{a} is independently selected from the group consisting of hydrogen, C₁-C₆-alkyl, C₁-C₆-haloalkyl, C₁-C₆-hydroxyalkyl, C₁-C₆-cyanoalkyl, C₂-C₆-alkenyl, C₂-C₆-haloalkenyl, C₂-C₆-hydroxyalkenyl, C₂-C₆-cyanoalkenyl, C₂-C₆-alkynyl, C₂-C₆-haloalkynyl, C₂-C₆-hydroxyalkynyl, C₂-C₆-cyanoalkynyl, carbocyclyl, heterocyclyl, aryl, heteroaryl, —C₁-C₆-alkyl-aryl, —C₁-C₆-alkyl-heterocyclyl, —C₁-C₆-alkyl-heteroaryl and —C₁-C₆-alkyl-C₁-C₆-alkoxy,
      wherein any carbocyclyl, heterocyclyl, aryl and heteroaryl group may be substituted by one or more substituents independently selected from the group consisting of halogen, hydroxy, oxo, cyano, C₁-C₆-alkyl, C₁-C₆-haloalkyl, C₁-C₆-alkoxy and C₁-C₆-haloalkoxy;
R2 is selected from the group consisting of hydrogen and C₁-C₆-alkyl,
   wherein said C₁-C₆-alkyl may be substituted by one or more substituents independently selected from the group consisting of halogen, hydroxy, cyano, mercapto, sulfinyl, sulfonyl, nitro, amino, C₁-C₆-alkoxy, C₁-C₆-haloalkoxy, C₁-C₆-alkylsulfanyl, C₁-C₆-alkylsulfinyl, C₁-C₆-alkylsulfonyl, —C(═O)—C₁-C₆-alkyl, —C(═O)—C₁-C₆-alkoxy, —C═O—C₁-C₆-alkylamino, —C═O—C₁-C₆-dialkylamino, carbocyclyl, heterocyclyl, aryl and heteroaryl,
      wherein said carbocyclyl, heterocyclyl, aryl and heteroaryl may be substituted by one or more substituents independently selected from the group consisting of halogen, hydroxy, oxo, cyano, C₁-C₆-alkyl, C₁-C₆-haloalkyl, C₁-C₆-alkoxy and C₁-C₆-haloalkoxy;
X is F or Cl;
n is 0, 1, 2, 3, 4 or 5;
R3 is independently selected from the group consisting of halogen, hydroxy, oxo, cyano, mercapto, sulfinyl, sulfonyl, nitro, C₁-C₆-alkyl, C₁-C₆-haloalkyl, C₁-C₆-hydroxyalkyl, C₁-C₆-cyanoalkyl, C₁-C₆-aminoalkyl, C₂-C₆-alkenyl, C₂-C₆-haloalkenyl, C₂-C₆-hydroxyalkenyl, C₂-C₆-cyanoalkenyl, C₂-C₆-alkynyl, C₂-C₆-haloalkynyl, C₂-C₆-hydroxyalkynyl, C₂-C₆-cyanoalkynyl, C₁-C₆-alkoxy, C₁-C₆-haloalkoxy, C₁-C₆-alkylsulfanyl, arylsulfanyl, C₁-C₆-alkylsulfinyl, arylsulfinyl, C₁-C₆-alkylsulfonyl, arylsulfonyl, amino, carbocyclyl, heterocyclyl, aryl, heteroaryl, —O-carbocyclyl, —O-heterocyclyl, —O-aryl, —O-heteroaryl, ═N(OR^{b}), —N(OR^{b})C(═O)OR^{b}, —N(OR^{b})C(═O)R^{b}, —SO₂N(R^{b})₂, —OC(═O)R^{b}, —N(R^{b})₂, —NR^{b}C(═O)OR^{b}, —NR^{b}C(═O)N(R^{b})₂, —NR^{b}C(═O)R^{b}, —NR^{b}C(═O)OR^{b}, —NR^{b}C(═S)R^{b}, —NR^{b}C(═S)N(R^{b})₂, —NR^{b}C(═NR^{b})R^{b}, —N═CR^{b}—N(R^{b})₂, —NR^{b}S(═O)₂R^{b}, —OC(═O)N(R^{b})₂, —C(═NR^{b})R^{b}, —C(═NOH)R^{b}, —C(═NOH)N(R^{b})₂, —C(═O)R^{b}, —C(═O)(OR^{b}), —C(═O)N(R^{b})₂, —C(═O)NR^{b}N(R^{b})₂, —C(═O)N(OR^{b})R^{b}, —C(═S)N(R^{b})₂, —C₁-C₆-(halo)alkyl-OR^{b}, —C₁-C₆-(halo)alkyl-O—C₁-C₆-(halo)alkyl, —C₁-C₆-(halo)alkyl-OC(═O)R^{b}, —C₁-C₆-(halo)alkyl-N(R^{b})₂, —C₁-C₆-(halo)alkyl-NR^{b}C(═O)OR^{b}, —C₁-C₆-(halo)alkyl-N(OR^{b})C(═O)OR^{b}, —C₁-C₆-(halo)alkyl-NR^{b}C(═O)N(R^{b})₂, —C₁-C₆-(halo)alkyl-NR^{b}C(═S)N(R^{b})₂, —C₁-C₆-(halo)alkyl-NR^{b}C(═O)R^{b}, —C₁-C₆-(halo)alkyl-NR^{b}C(═S)R^{b}, —C₁-C₆-(halo)alkyl-NR^{b}C(═S)N(R^{b})₂, —C₁-C₆-(halo)alkyl-NR^{b}C(═NR^{b})R^{b}, —C₁-C₆-(halo)alkyl-NR^{b}S(═O)₂R^{b}, —C₁-C₆-(halo)alkyl-N(OR^{b})C(═O)R^{b}, —C₁-C₆-(halo)alkyl-carbocyclyl, —C₁-C₆-(halo)alkyl-aryl, —C₁-C₆-(halo)alkyl-heterocyclyl, —C₁-C₆-(halo)alkyl-heteroaryl, —C₁-C₆-(halo)alkyl-O-carbocyclyl, —C₁-C₆-(halo)alkyl-O-aryl, —C₁-C₆-(halo)alkyl-O-heterocyclyl, —C₁-C₆-(halo)alkyl-O-heteroaryl, —C₁-C₆-(halo)alkyl-C(═O)R^{b}, —C₁-C₆-(halo)alkyl-C(═O)OR^{b}, —C₁-C₆-(halo)alkyl-C(═O)N(R^{b})₂, —C₁-C₆-(halo)alkyl-C(═O)N(OR^{b})R^{b}, —C₁-C₆-(halo)alkyl-C(═S)N(R^{b})₂, —C₁-C₆-(halo)alkyl-OC(═O)N(R^{b})₂, —C₁-C₆-(halo)alkyl-S—R^{b}, —C₁-C₆-(halo)alkyl-S(═O)₂R^{b}, —C₁-C₆-(halo)alkyl-S(═O)₂N(R^{b})₂, —C₁-C₆-(halo)alkyl-C(═NR^{b})R^{b} and —C₁-C₆-alkyl-R^{c} wherein the C₁-C₆-alkyl in said C₁-C₆-alkyl-R^{c} is substituted with two substituents on a same carbon atom that form together with the carbon atom to which they are attached a carbocyclyl (e.g. a cyclopropyl),
   wherein any carbocyclyl, heterocyclyl, aryl and heteroaryl group may be substituted with one or more R³¹ substituents that may be the same or different,
      wherein R³¹ is independently selected from the group consisting of halogen, hydroxy, oxo, cyano, mercapto, sulfinyl, sulfonyl, nitro, C₁-C₆-alkyl, C₁-C₆-haloalkyl, C₁-C₆-hydroxyalkyl, C₁-C₆-cyanoalkyl, C₂-C₆-alkenyl, C₂-C₆-haloalkenyl, C₂-C₆-hydroxyalkenyl, C₂-C₆-cyanoalkenyl, C₂-C₆-alkynyl, C₂-C₆-haloalkynyl, C₂-C₆-hydroxyalkynyl, C₂-C₆-cyanoalkynyl, C₁-C₆-alkoxy, C₁-C₆-haloalkoxy, C₁-C₆-alkylsulfanyl, arylsulfanyl, C₁-C₆-alkylsulfinyl, arylsulfinyl, C₁-C₆-alkylsulfonyl, arylsulfonyl, amino, carbocyclyl, heterocyclyl, aryl, heteroaryl, —O-carbocyclyl, —O-heterocyclyl, —O-aryl, —O-heteroaryl, ═N(OR³¹¹), —N(OR³¹¹)C(═O)OR³¹¹, —N(OR³¹¹)C(═O)R³¹¹, —SO₂N(R³¹¹)₂, —OC(═O)R³¹¹, —N(R³¹¹)₂, —NR³¹¹C(═O)OR³¹¹, —NR³¹¹C(═O)N(R³¹¹)₂, —NR³¹¹C(═O)R³¹¹, —NR³¹¹C(═S)R³¹¹, —NR³¹¹C(═S)N(R³¹¹)₂, —NR³¹¹C(═NR³¹¹)R³¹¹, —N═C—N(R³¹¹)₂, —NR³¹¹S(═O)₂R³¹¹, —OC(═O)N(R³¹¹)₂, —C(═NR³¹¹)R³¹¹, —C(═NOH)R³¹¹, —C(═O)R³¹¹, —C(═O)(OR³¹¹), —C(═O)N(R³¹¹)₂, —C(═O)NR³¹¹N(R³¹¹)₂, —C(═O)N(OR³¹¹)R³¹¹, —C(═S)N(R³¹¹)₂, —C₁-C₆-alkyl-OR³¹¹, —C₁-C₆-alkyl-N(R³¹¹)₂, —C₁-C₆-alkyl-NR³¹¹C(═O)OR³¹¹, —C₁-C₆-alkyl-NR³¹¹C(═O)N(R³¹¹)₂, —C₁-C₆-alkyl-NR³¹¹C(═S)N(R³¹¹)₂, —C₁-C₆-alkyl-NR³¹¹C(═O)R³¹¹, —C₁-C₆-alkyl-NR³¹¹C(═S)R³¹¹, —C₁-C₆-alkyl-NR³¹¹C(═S)N(R³¹¹)₂, —C₁-C₆-alkyl-NR³¹¹C(═NR³¹¹)R³¹¹, —C₁-C₆-alkyl-NR³¹¹S(═O)₂R³¹¹, —C₁-C₆-alkyl-N(OR³¹¹)C(═O)R³¹¹, —C₁-C₆-alkyl-carbocyclyl, —C₁-C₆-alkyl-aryl, —C₁-C₆-alkyl-heterocyclyl, —C₁-C₆-alkyl-heteroaryl, —C₁-C₆-alkyl-C(═O)R³¹¹, —C₁-C₆-alkyl-C(═O)OR³¹¹, —C₁-C₆-alkyl-C(═O)N(R³¹¹)₂, —C₁-C₆-alkyl-C(═O)N(OR³¹¹)R³¹¹, —C₁-C₆-alkyl-C(═S)N(R³¹¹)₂, —C₁-C₆-alkyl-OC(═O)N(R³¹¹)₂, —C₁-C₆-alkyl-S(═O)₂R³¹¹, —C₁-C₆-alkyl-S(═O)₂N(R³¹¹)₂ and —C₁-C₆-alkyl-C(═NR³¹¹)R³¹¹, wherein R³¹¹ is independently hydrogen or C₁-C₆-alkyl,
   wherein R^{c} is selected from the group consisting of —OR^{b}, —OC(═O)R^{b}, —N(R^{b})₂, —NR^{b}C(═O)OR^{b}, —N(OR^{b})C(═O)OR^{b}, —NR^{b}C(═O)N(R^{b})₂, —NR^{b}C(═S)N(R^{b})₂, —NR^{b}C(═O)R^{b}, —NR^{b}C(═S)R^{b}, —NR^{b}C(═S)N(R^{b})₂, —NR^{b}C(═NR^{b})R^{b}, —NR^{b}S(═O)₂R^{b}, —N(OR^{b})C(═O)R^{b}, -carbocyclyl, -aryl, -heterocyclyl, -heteroaryl, —O-carbocyclyl, —O-aryl, —O-heterocyclyl, —O-heteroaryl, —C(═O)R^{b}, —C(═O)OR^{b}, —C(═O)N(R^{b})₂, —C(═O)N(OR^{b})R^{b}, —C(═S)N(R^{b})₂, —OC(═O)N(R^{b})₂, —S—R^{b}, —S(═O)₂R^{b}, —S(═O)₂N(R^{b})₂ and —C(═NR^{b})R^{b},
      wherein any carbocyclyl, heterocyclyl, aryl and heteroaryl group may be substituted by one or more R^{c1} substituents that may be the same or different,
         R^{c1} is independently selected from the group consisting of halogen, hydroxy, oxo, cyano, mercapto, sulfinyl, sulfonyl, nitro, C₁-C₆-alkyl, C₁-C₆-haloalkyl, C₁-C₆-hydroxyalkyl, C₁-C₆-cyanoalkyl, C₂-C₆-alkenyl, C₂-C₆-haloalkenyl, C₂-C₆-hydroxyalkenyl, C₂-C₆-cyanoalkenyl, C₂-C₆-alkynyl, C₂-C₆-haloalkynyl, C₂-C₆-hydroxyalkynyl, C₂-C₆-cyanoalkynyl, C₁-C₆-alkoxy, C₁-C₆-haloalkoxy, C₁-C₆-alkylsulfanyl, arylsulfanyl, C₁-C₆-alkylsulfinyl, arylsulfinyl, C₁-C₆-alkylsulfonyl, arylsulfonyl, amino, carbocyclyl, heterocyclyl, aryl, heteroaryl, —O-carbocyclyl, —O— heterocyclyl, —O-aryl, —O-heteroaryl, ═N(OR¹¹), —N(OR^{c11})C(═O)OR^{c11}, —N(OR^{c11})C(═O)R^{c11}, —SO₂N(R^{c11})₂, —OC(═O)R^{c11}, —N(R^{c11})₂, —NR^{c11}C(═O)OR^{c11}, —NR^{c11}C(═O)N(R^{c11})₂, —NR^{c11}C(═O)R^{c11}, —NR^{c11}C(═S)R^{c11}, —NR^{c11}C(═S)N(R^{c11})₂, —NR^{c11}C(═NR^{c11})R^{c11}, —N═C—N(R^{c11})₂, —NR^{c11}S(═O)₂R^{c11}, —OC(═O)N(R^{c11})₂, —C(═NR^{c11})R^{c11}, —C(═NOH)R^{c11}, —C(═O)R^{c11}, —C(═O)(OR^{c11}), —C(═O)N(R^{c11})₂, —C(═O)NR^{c11}N(R^{c11})₂, —C(═O)N(OR^{c11})R^{c11}, —C(═S)N(R^{c11})₂, —C₁-C₆-alkyl-OR^{c11}, —C₁-C₆-alkyl-N(R^{c11})₂, —C₁-C₆-alkyl-NR^{c11}C(═O)OR^{c11}, —C₁-C₆-alkyl-NR^{c11}C(═O)N(R^{c11})₂, —C₁-C₆-alkyl-NR^{c11}C(═S)N(R^{c11})₂, —C₁-C₆-alkyl-NR^{c11}C(═O)R^{c11}, —C₁-C₆-alkyl-NR^{c11}C(═S)R^{c11}, —C₁-C₆-alkyl-NR^{c11}C(═S)N(R^{c11})₂, —C₁-C₆-alkyl-NR^{c11}C(═NR^{c11})R^{c11}, —C₁-C₆-alkyl-NR^{b11}S(═O)₂R^{c11}, —C₁-C₆-alkyl-N(OR^{c11})C(═O)R^{c11}, —C₁-C₆-alkyl-carbocyclyl, —C₁-C₆-alkyl-aryl, —C₁-C₆-alkyl-heterocyclyl, —C₁-C₆-alkyl-heteroaryl, —C₁-C₆-alkyl-C(═O)R^{c11}, —C₁-C₆-alkyl-C(═O)OR^{c11}, —C₁-C₆-alkyl-C(═O)N(R^{c11})₂, —C₁-C₆-alkyl-C(═O)N(OR^{c11})R^{c11}, —C₁-C₆-alkyl-C(═S)N(R^{c11})₂, —C₁-C₆-alkyl-OC(═O)N(R^{c11})₂, —C₁-C₆-alkyl-S(═O)₂R^{c11}, —C₁-C₆-alkyl-S(═O)₂N(R^{c11})₂ and —C₁-C₆-alkyl-C(═NR^{c11})R^{c11}, wherein R^{c11} is independently hydrogen or C₁-C₆-alkyl,
   wherein R^{b} is independently selected from the group consisting of hydrogen, halogen, hydroxy, cyano, mercapto, sulfinyl, sulfonyl, nitro, C₁-C₆-alkyl, C₁-C₆-haloalkyl, C₁-C₆-hydroxyalkyl, C₁-C₆-cyanoalkyl, C₂-C₆-alkenyl, C₂-C₆-haloalkenyl, C₂-C₆-hydroxyalkenyl, C₂-C₆-cyanoalkenyl, C₂-C₆-alkynyl, C₂-C₆-haloalkynyl, C₂-C₆-hydroxyalkynyl, C₂-C₆-cyanoalkynyl, C₁-C₆-alkoxy, C₁-C₆-haloalkoxy, C₁-C₆-alkylsulfanyl, arylsulfanyl, C₁-C₆-alkylsulfinyl, arylsulfinyl, C₁-C₆-alkylsulfonyl, arylsulfonyl, amino, C₁-C₆-alkylamino, carbocyclyl, heterocyclyl, aryl, heteroaryl, —O— carbocyclyl, —O-heterocyclyl, —O-aryl, —O-heteroaryl, —C₁-C₆-alkyl-carbocyclyl, —C₁-C₆-alkyl-aryl, —C₁-C₆-alkyl-heterocyclyl, —C₁-C₆-alkyl-heteroaryl, —C₁-C₆-alkyl-O-carbocyclyl, —C₁-C₆-alkyl-O-aryl, —C₁-C₆-alkyl-O-heterocyclyl, —C₁-C₆-alkyl-O-heteroaryl and —C₁-C₆-alkyl-C₁-C₆-alkoxy,
      wherein any carbocyclyl, heterocyclyl, aryl and heteroaryl group may be substituted by one or more R^{b1} substituents that may be the same or different;
         R^{b1} is independently selected from the group consisting of halogen, hydroxy, oxo, cyano, mercapto, sulfinyl, sulfonyl, nitro, C₁-C₆-alkyl, C₁-C₆-haloalkyl, C₁-C₆-hydroxyalkyl, C₁-C₆-cyanoalkyl, C₂-C₆-alkenyl, C₂-C₆-haloalkenyl, C₂-C₆-hydroxyalkenyl, C₂-C₆-cyanoalkenyl, C₂-C₆-alkynyl, C₂-C₆-haloalkynyl, C₂-C₆-hydroxyalkynyl, C₂-C₆-cyanoalkynyl, C₁-C₆-alkoxy, C₁-C₆-haloalkoxy, C₁-C₆-alkylsulfanyl, arylsulfanyl, C₁-C₆-alkylsulfinyl, arylsulfinyl, C₁-C₆-alkylsulfonyl, arylsulfonyl, amino, carbocyclyl, heterocyclyl, aryl, heteroaryl, —O-carbocyclyl, —O-heterocyclyl, —O-aryl, —O— heteroaryl, ═N(OR^{b11}), —N(OR^{b11})C(═O)OR^{b11}, —N(OR^{b11})C(═O)R^{b11}, —SO₂N(R^{b11})₂, —OC(═O) R^{b11}, —N(R^{b11})₂, —NR^{b11}C(═O)OR^{b11}, —NR^{b11}C(═O)N(R^{b11})₂, —NR^{b11}C(═O)R^{b11}, —NR^{b11}C(═S)R^{b11}, —NR^{b11}C(═S)N(R^{b11})₂, —NR^{b11}C(═NR^{b11})R^{b11}, —N═C—N(R^{b11})₂, —NR^{b11}S(═O)₂R^{b11}, —OC(═O)N(R^{b11})₂, —C(═NR^{b11})R^{b11}, —C(═NOH)R^{b11}, —C(═O)R^{b11}, —C(═O)(OR^{b11}), —C(═O)N(R^{b11})₂, —C(═O)NR^{b11}N(R^{b11})₂, —C(═O)N(OR^{b11})R^{b11}, —C(═S)N(R^{b11})₂, —C₁-C₆-alkyl-OR^{b11}, —C₁-C₆-alkyl-N(R^{b11})₂, —C₁-C₆-alkyl-NR^{b11}C(═O)OR^{b11}, —C₁-C₆-alkyl-NR^{b11}C(═O)N(R^{b11})₂, —C₁-C₆-alkyl-NR^{b11}C(═S)N(R^{b11})₂, —C₁-C₆-alkyl-NR^{b11}C(═O)R^{b11}, —C₁-C₆-alkyl-NR^{b11}C(═S)R^{b11}, —C₁-C₆-alkyl-NR^{b11}C(═S)N(R^{b11})₂, —C₁-C₆-alkyl-NR^{b11}C(═NR^{b11})R^{b11}, —C₁-C₆-alkyl-NR^{b11}S(═O)₂R^{b11}, —C₁-C₆-alkyl-N(OR^{b11})C(═O)R^{b11}, —C₁-C₆-alkyl-carbocyclyl, —C₁-C₆-alkyl-aryl, —C₁-C₆-alkyl-heterocyclyl, —C₁-C₆-alkyl-heteroaryl, —C₁-C₆-alkyl-C(═O)R^{b11}, —C₁-C₆-alkyl-C(═O)OR^{b11}, —C₁-C₆-alkyl-C(═O)N(R^{b11})₂, —C₁-C₆-alkyl-C(═O)N(OR^{b11})R^{b11}, —C₁-C₆-alkyl-C(═S)N(R^{b11})₂, —C₁-C₆-alkyl-OC(═O)N(R^{b11})₂, —C₁-C₆-alkyl-S(═O)₂R^{b11}, —C₁-C₆-alkyl-S(═O)₂N(R^{b11})₂ and —C₁-C₆-alkyl-C(═NR^{b11})R^{b11}, wherein R^{b11} is independently hydrogen or C₁-C₆-alkyl;
            provided compound of formula (I) is not
   (a) 1-{4-[5-methyl-5-(trifluoromethyl)-4,5-dihydro-1,2,4-oxadiazol-3-yl]phenyl}methanamine [510772-18-6];
   (b) di-tert-butyl{4-[5-methyl-5-(trifluoromethyl)-4,5-dihydro-1,2,4-oxadiazol-3-yl]benzyl}-2-imidodicarbonate [510772-17-5];
   (c) 1-{4-[5,5-bis(trifluoromethyl)-4,5-dihydro-1,2,4-oxadiazol-3-yl]phenyl}methanamine [510772-14-2];
   (d) 2-{6-[3-amino-5-(trifluoromethyl)phenyl]-3-(isopropylamino)-2-oxopyrazin-1(2H)-yl}-N-{4-[5-methyl-5-(trifluoromethyl)-4,5-dihydro-1,2,4-oxadiazol-3-yl]benzyl}acetamide [510770-08-8];
   (e) 2-{6-[3-amino-5-(trifluoromethyl)phenyl]-3-(isopropylamino)-2-oxopyrazin-1(2H)-yl}-N-{4-[5,5-bis(trifluoromethyl)-4,5-dihydro-1,2,4-oxadiazol-3-yl]benzyl}acetamide [510770-03-3];
   (f) phenyl[3-phenyl-5,5-bis(trifluoromethyl)-1,2,4-oxadiazol-4(5H)-yl]methanone [21111-45-5]; and
   (g) 3-phenyl-5,5-bis(trifluoromethyl)-4,5-dihydro-1,2,4-oxadiazole [21111-43-3].

Compounds (d) and (e) are known as prodrugs useful for selective inhibition of serine protease of the coagulation cascade. They may be used for preventing and treating thrombotic conditions in mammals (WO2003/028729). Compounds (a), (b) and (c) are known as reactants in the preparation of some of these serine protease inhibitors (WO2003/028729). Compounds (f) and (g) are disclosed in the literature without reference to any agricultural use (Simonyan, L., A. et al, Izvestiya Akademli Nauk SSSR, Seriya Khimicheskaya (1968), (8), 1916-18).

Not encompassed herein are compounds resulting from combinations which are against natural laws and which the person skilled in the art would therefore exclude based on his/her expert knowledge. For instance, ring structures having three or more adjacent oxygen atoms are excluded.

The compound of formula (I) can suitably be in its free form, salt form, N-oxides form or solvate form (e.g. hydrate).

Depending on the nature of the substituents, the compound of formula (I) may be present in the form of different stereoisomers. These stereoisomers are, for example, enantiomers, diastereomers, atropisomers or geometric isomers. Accordingly, the invention encompasses both pure stereoisomers and any mixture of these isomers. Where a compound can be present in two or more tautomer forms in equilibrium, reference to the compound by means of one tautomeric description is to be considered to include all tautomer forms.

Any of the compounds of the present invention can also exist in one or more geometric isomer forms depending on the number of double bonds in the compound. Geometric isomers by nature of substituents about a double bond or a ring may be present in cis (=Z-) or trans (=E-) form. The invention thus relates equally to all geometric isomers and to all possible mixtures, in all proportions.

Depending on the nature of the substituents, the compound of formula (I) may be present in the form of the free compound and/or a salt thereof, such as an agrochemically active salt.

Agrochemically active salts include acid addition salts of inorganic and organic acids well as salts of customary bases. Examples of inorganic acids are hydrohalic acids, such as hydrogen fluoride, hydrogen chloride, hydrogen bromide and hydrogen iodide, sulfuric acid, phosphoric acid and nitric acid, and acidic salts, such as sodium bisulfate and potassium bisulfate. Useful organic acids include, for example, formic acid, carbonic acid and alkanoic acids such as acetic acid, trifluoroacetic acid, trichloroacetic acid and propionic acid, and also glycolic acid, thiocyanic acid, lactic acid, succinic acid, citric acid, benzoic acid, cinnamic acid, oxalic acid, saturated or mono- or diunsaturated fatty acids having 6 to 20 carbon atoms, alkylsulphuric monoesters, alkylsulphonic acids (sulphonic acids having straight-chain or branched alkyl radicals having 1 to 20 carbon atoms), arylsulphonic acids or aryldisulphonic acids (aromatic radicals, such as phenyl and naphthyl, which bear one or two sulphonic acid groups), alkylphosphonic acids (phosphonic acids having straight-chain or branched alkyl radicals having 1 to 20 carbon atoms), arylphosphonic acids or aryldiphosphonic acids (aromatic radicals, such as phenyl and naphthyl, which bear one or two phosphonic acid radicals), where the alkyl and aryl radicals may bear further substituents, for example p-toluenesulphonic acid, salicylic acid, p-aminosalicylic acid, 2-phenoxybenzoic acid, 2-acetoxybenzoic acid, etc.

Solvates of the compounds of the invention or their salts are stoichiometric compositions of the compounds with solvents.

The compounds of the invention may exist in multiple crystalline and/or amorphous forms. Crystalline forms include unsolvated crystalline forms, solvates and hydrates.

Compounds of formula (I) are referred herein as “active ingredients”.

In the above formula, A is preferably selected from the group consisting of 9- or 10-membered bicyclic heterocyclyl, C₆-C₁₀-aryl, 5- or 6-membered monocyclic heteroaryl and 9- or 10-membered bicyclic heteroaryl.

In the above formula, A is more preferably selected from the group consisting of 10-membered bicyclic heterocycle comprising one nitrogen atom (e.g. dihydroisoquinolinyl, tetrahydroquinolinyl, tetrahydroisoquinolinyl), C₆-C₁₀-aryl (e.g. phenyl, naphthyl), 5- or 6-membered monocyclic heteroaryl comprising one or two nitrogen atoms (e.g. pyridinyl, pyrimidinyl) and 9- or 10-membered bicyclic heteroaryl comprising one or two nitrogen atom (e.g. indolyl, benzimidazolyl, indazolyl).

In some embodiments, in the above formula, A is selected from the group consisting of dihydroisoquinolinyl, tetrahydroquinolinyl, tetrahydroisoquinolinyl, phenyl, naphthyl, indolyl, benzimidazolyl and indazolyl.

In the above formula (I), when R1 is a C₁-C₆-alkyl substituted by a carbocyclyl, it may be preferred that said carbocyclyl be a C₃-C₄-carbocyclyl.

In the above formula (I), when R1 is a C₁-C₆-alkyl substituted by a heterocyclyl, it may be preferred that said heterocyclyl be a 3- to 6-membered heterocyclyl.

In the above formula (I), when R1 is a C₁-C₆-alkyl substituted by an aryl, it may be preferred that said aryl be a C₁-C₆-aryl.

In the above formula (I), when R1 is a C₁-C₆-alkyl substituted by a heteroaryl, it may be preferred that said heteroaryl be a 5- or 6-membered heteroaryl.

In the above formula (I), when R2 is a C₁-C₆-alkyl substituted by a carbocyclyl, it may be preferred that said carbocyclyl be a C₃-C₄-carbocyclyl.

In the above formula (I), when R2 is a C₁-C₆-alkyl substituted by a heterocyclyl, it may be preferred that said heterocyclyl be a 3- to 6-membered heterocyclyl.

In the above formula (I), when R2 is a C₁-C₆-alkyl substituted by an aryl, it may be preferred that said aryl be a C₁-C₆-aryl.

In the above formula (I), when R2 is a C₁-C₆-alkyl substituted by a heteroaryl, it may be preferred that said heteroaryl be a 5- or 6-membered heteroaryl.

In the above formula (I), R^{a} is preferably a C₁-C₆-alkyl.

In the above formula (I), R1 is preferably selected from the group consisting of hydrogen, C₁-C₆-alkyl and —C(═O)R^{a} wherein R^{a} is as described herein, preferably wherein R^{a} is C₁-C₆-alkyl, more preferably R1 is hydrogen or —C(═O)R^{a} wherein R^{a} is as described herein, preferably wherein R^{a} is C₁-C₆-alkyl.

In the above formula (I), R2 is preferably hydrogen or C₁-C₆-alkyl, more preferably R2 is hydrogen.

In the above formula (I), R³¹ is preferably selected from the group consisting of halogen, oxo, C₁-C₆-alkyl, C₁-C₆-alkoxy, heteroaryl, —NR³¹¹C(═O)OR³¹¹ and —C(═O)(OR³¹¹) with R³¹¹ being hydrogen or C₁-C₆-alkyl.

In some embodiments, in the above formula (I), R³¹ is selected from the group consisting of halogen, oxo and C₁-C₆-alkyl.

In the above formula (I), R^{b} is preferably selected from the group consisting of hydrogen, C₁-C₆-alkyl, C₂-C₆-alkynyl, C₁-C₆-alkylsulfanyl, carbocyclyl (e.g. cyclopropyl, hexyl), aryl (e.g. phenyl), heterocyclyl, heteroaryl, —C₁-C₆-alkyl-carbocyclyl, —C₁-C₆-alkyl-O-aryl and —C₁-C₆-alkyl-C₁-C₆-alkoxy, wherein any carbocyclyl, aryl, heterocyclyl and heteroaryl group may be substituted by one or more R^{b1} substituents as described herein, preferably wherein R^{b1} is halogen or C₁-C₆-alkyl.

In some embodiments, in the above formula (I), R^{b} is selected from the group consisting of hydrogen, C₁-C₆-alkyl, C₁-C₆-alkylsulfanyl, carbocyclyl, aryl, heterocyclyl, heteroaryl and —C₁-C₆-alkyl-carbocyclyl, wherein any carbocyclyl, aryl, heterocyclyl and heteroaryl group may be substituted by one or more R^{b1} substituents as described herein, preferably wherein R^{b1} is halogen or C₁-C₆-alkyl.

In the above formula (I), R3 is preferably selected from the group consisting of hydroxy, halogen, oxo, C₁-C₆-alkyl, C₁-C₆-alkylsulfanyl, arylsulfanyl, arylsulfinyl, C₁-C₆-alkylsulfonyl, arylsulfonyl, carbocyclyl, heterocyclyl, aryl, heteroaryl, —NR^{b}C(═O)R^{b}, —OC(═O)R^{b}, —N(R^{b})₂, —NR^{b}C(═O)OR^{b}, —N═CR^{b}—N(R^{b})₂, —OC(═O)N(R^{b})₂, —C(═NOH)R^{b}, —C(═NOH)N(R^{b})₂, —C(═O)R^{b}, —C(═O)(OR^{b}), —C(═O)N(R^{b})₂, —C(═O)NR^{b}N(R^{b})₂, —C(═S)N(R^{b})₂, —C₁-C₆-(halo)alkyl-O—C₁-C₆-(halo)alkyl, —C₁-C₆-(halo)alkyl-NR^{b}C(═O)OR^{b}, —C₁-C₆-(halo)alkyl-N(OR^{b})C(═O)OR^{b}, —C₁-C₆-(halo)alkyl-NR^{b}C(═O)R^{b}, —C₁-C₆-(halo)alkyl-N(OR^{b})C(═O)R^{b}, —C₁-C₆-(halo)alkyl-aryl, —C₁-C₆-(halo)alkyl-C(═O)N(R^{b})₂, —C₁-C₆-alkyl-R^{c} wherein the C₁-C₆-alkyl in said C₁-C₆-alkyl-R^{c} is substituted with two substituents on a same carbon atom that form together with the carbon atom to which they are attached a carbocyclyl (e.g. a cyclopropyl), wherein any of the previously cited carbocyclyl, heterocyclyl, aryl and heteroaryl groups may be substituted with one or more R³¹ substituents as described herein (preferably R³¹ is independently selected from the group consisting halogen, oxo, C₁-C₆-alkyl, C₁-C₆-alkoxy, heteroaryl, —NR³¹¹C(═O)OR³¹1 and —C(═O)(OR³¹¹) with R³¹1 being hydrogen or C₁-C₆-alkyl), and wherein R^{b} and R are as described herein, preferably wherein R^{b} is selected from the group consisting of hydrogen, C₁-C₆-alkyl, C₂-C₆-alkynyl, C₁-C₆-alkylsulfanyl, carbocyclyl, aryl, —C₁-C₆-alkyl-carbocyclyl, —C₁-C₆-alkyl-O-aryl and —C₁-C₆-alkyl-C₁-C₆-alkoxy (wherein any of the previously cited carbocyclyl or aryl, group may be substituted by one or more R^{b1} substituents as described herein, preferably wherein R^{b1} is halogen or C₁-C₆-alkyl), preferably R^{c} is —NR^{b}C(═O)R^{b} with R^{b} being a C₁-C₆-alkyl.

In some embodiments, in the above formula (I), R3 is selected from the group consisting of hydroxy, oxo, C₁-C₆-alkyl, C₁-C₆-alkylsulfanyl, arylsulfanyl, arylsulfinyl, C₁-C₆-alkylsulfonyl, arylsulfonyl, carbocyclyl, heterocyclyl, aryl, heteroaryl, —OC(═O)R^{b}, —N(R^{b})₂, —NR^{b}C(═O)OR^{b}, —OC(═O)N(R^{b})₂, —C(═NOH)R^{b}, —C(═NOH)N(R^{b})₂, —C(═O)R^{b}, —C(═O)(OR^{b}), —C(═O)N(R^{b})₂, —C(═O)NR^{b}N(R^{b})₂, —C(═S)N(R^{b})₂, —C₁-C₆-(halo)alkyl-NR^{b}C(═O)OR^{b}, —C₁-C₆-(halo)alkyl-N(OR^{b})C(═O)OR^{b}, —C₁-C₆-(halo)alkyl-NR^{b}C(═O)R^{b}, —C₁-C₆-(halo)alkyl-N(OR^{b})C(═O)R^{b} and —C₁-C₆-(halo)alkyl-aryl wherein any carbocyclyl, heterocyclyl, aryl and heteroaryl group may be substituted with one or more R³¹ substituents as described herein and wherein R^{b} is as described herein, preferably wherein R^{b} is selected from the group consisting of hydrogen, C₁-C₆-alkyl, C₁-C₆-alkoxy, C₁-C₆-alkylsulfanyl, carbocyclyl, heterocyclyl, aryl and —C₁-C₆-alkyl-carbocyclyl, wherein any carbocyclyl, aryl and heterocyclyl group may be substituted by one or more R^{b1} substituents as described herein.

In some embodiments, in the above formula (I), R3 is selected from the group consisting of halogen; oxo; C₁-C₆-alkyl; C₁-C₆-alkylsulfanyl; arylsulfanyl that may be substituted with one or more R³¹ substituents as described herein, preferably wherein R³¹ is C₁-C₆-alkoxy; arylsulfinyl that may be substituted with one or more R³¹ substituents as described herein, preferably wherein R³¹ is C₁-C₆-alkoxy; arylsulfonyl that may be substituted with one or more R³¹ substituents as described herein, preferably wherein R³¹ is C₁-C₆-alkoxy; heterocyclyl that may be substituted with one or more R³¹ substituents as described herein, preferably wherein R³¹ is oxo or —C(═O)(OR³¹¹) with R³¹¹ being a C₁-C₆-alkyl; aryl that may be substituted with one or more R³¹ substituents as described herein, preferably wherein R³¹ is —NR³¹¹C(═O)OR³¹¹ with R³¹¹ being hydrogen or C₁-C₆-alkyl; heteroaryl that may be substituted with one or more R³¹ substituents as described herein, preferably wherein R³¹ is C₁-C₆-alkyl; —NR^{b}C(═O)R^{b} wherein R^{b} is as disclosed herein, preferably wherein R^{b} is hydrogen, C₁-C₆-alkyl, carbocyclyl (e.g. cyclopropyl) that may be substituted with with one or more R^{b1} substituents as described herein, preferably wherein R^{b1} is C₁-C₆-alkyl or R^{b} is —C₁-C₆-alkyl-O-aryl (e.g. —C₁-C₆-alkyl-O-phenyl) wherein said aryl may be substituted with one or more R^{b1} substituents as described herein, preferably wherein R^{b1} is halogen; —NR^{b}C(═O)OR^{b} wherein R^{b} is as disclosed herein, preferably wherein R^{b} is independently hydrogen or C₁-C₆-alkyl; —N═CR^{b}—N(R^{b})₂ wherein R^{b} is as disclosed herein, preferably wherein R^{b} is independently hydrogen or C₁-C₆-alkyl; —OC(═O)N(R^{b})₂ wherein R^{b} is as disclosed herein, preferably wherein R^{b} is independently C₁-C₆-alkyl, aryl (e.g. phenyl) or carbocyclyl (e.g. hexyl); —C(═NOH)R^{b} wherein R^{b} is as disclosed herein, preferably wherein R^{b} is hydrogen or C₁-C₆-alkylsulfanyl; —C(═O)R^{b} wherein R^{b} is as disclosed herein, preferably wherein R^{b} is C₁-C₆-alkyl; —C(═O)(OR^{b}) wherein R^{b} is as disclosed herein, preferably wherein R^{b} is C₁-C₆-alkyl; —C(═O)N(R^{b})₂ wherein R^{b} is as disclosed herein, preferably wherein R^{b} is independently hydrogen, —C₁-C₆-alkyl-carbocyclyl, —C₁-C₆-alkyl-C₁-C₆-alkoxy, C₁-C₆-alkyl, carbocyclyl (e.g. cyclopropyl) that may be substituted with one or more R^{b1} substituents as described herein, preferably wherein R^{b1} is C₁-C₆-alkyl or R^{b} is aryl (e.g. phenyl) that may be substituted with one or more R^{b1} substituents as described herein, preferably wherein R^{b1} is halogen; —C(═O)NR^{b}N(R^{b})₂ wherein R^{b} is as disclosed herein, preferably wherein R^{b} is hydrogen or C₁-C₆-alkyl; —C(═S)N(R^{b})₂ wherein R^{b} is as disclosed herein, preferably wherein R^{b} is independently hydrogen, C₁-C₆-alkyl or —C₁-C₆-alkyl-carbocyclyl; —C₁-C₆-(halo)alkyl-O—C₁-C₆-(halo)alkyl; —C₁-C₆-(halo)alkyl-NR^{b}C(═O)OR^{b} wherein R^{b} is as disclosed herein, preferably wherein R^{b} is independently hydrogen or C₁-C₆-alkyl; —C₁-C₆-(halo)alkyl-N(OR^{b})C(═O)OR^{b} wherein R^{b} is as disclosed herein, preferably wherein R^{b} is independently C₁-C₆-alkyl or aryl (e.g. phenyl) that may be substituted with one or more R^{b1} substituents as described herein, preferably wherein R^{b1} is halogen; —C₁-C₆-(halo)alkyl-NR^{b}C(═O)R^{b} wherein R^{b} is as disclosed herein, preferably wherein R^{b} is independently hydrogen or carbocyclyl (e.g. cyclopropyl); —C₁-C₆-(halo)alkyl-N(OR^{b})C(═O)R^{b} wherein R^{b} is as disclosed herein, preferably wherein R^{b} is independently C₁-C₆-alkyl or carbocyclyl (e.g. cyclopropyl); —C₁-C₆-(halo)alkyl-aryl; —C₁-C₆-(halo)alkyl-C(═O)N(R^{b})₂ wherein R^{b} is as disclosed herein, preferably wherein R^{b} is independently hydrogen or —C₂-C₁₀-alkynyl; —C₁-C₆-alkyl-R^{c} wherein the C₁-C₆-alkyl in said C₁-C₆-alkyl-R^{c} is substituted with two substituents on a same carbon atom that form together with the carbon atom to which they are attached a carbocyclyl (e.g. a cyclopropyl) and wherein R^{c} is as disclosed herein, preferably wherein R^{c} is —NR^{b}C(═O)R^{b} with R^{b} being a C₁-C₆-alkyl.

In some embodiments, in the above formula (I), R3 is selected from the group consisting of C₁-C₆-alkyl; C₁-C₆-alkylsulfanyl; C₁-C₆-alkylsulfonyl; heterocyclyl that may be substituted by oxo; —C₁-C₆-alkyl-aryl; oxo; N(R^{b})₂ wherein R^{b} is as disclosed herein, preferably wherein R^{b} is hydrogen and aryl (e.g. phenyl) that may be substituted with halogen, or R^{b} is hydrogen and heteroaryl; —OC(═O)N(R^{b})₂ wherein R^{b} is as disclosed herein, preferably wherein R^{b} is independently selected from the group consisting of hydrogen, C₁-C₆-alkyl, carbocyclyl and aryl; —NR^{b}C(═O)OR^{b} wherein R^{b} is as disclosed herein, preferably wherein R^{b} is independently selected from the group consisting of hydrogen and C₁-C₆-alkyl; —C(═S)N(R^{b})₂ wherein R^{b} is as disclosed herein, preferably wherein R^{b} is independently selected from the group consisting of hydrogen, C₁-C₆-alkyl and —C₁-C₆-alkyl-C₃-C₈-cycloalkyl; —C₁-C₆-alkyl-N(OR^{b})C(═O)R^{b} wherein R^{b} is as disclosed herein, preferably wherein R^{b} are independently selected from the group consisting of C₁-C₆-alkyl and C₃-C₃-cycloalkyl; —C₁-C₆-alkyl-NR^{b}C(═O)OR^{b} wherein R^{b} is as disclosed herein, preferably wherein R^{b} are independently selected from the group consisting of hydrogen, C₁-C₆-alkoxy and heterocyclyl; —C₁-C₆-alkyl-NR^{b}C(═O)R^{b} wherein R^{b} is as disclosed herein, preferably wherein R^{b} are independently selected from the group consisting of hydrogen and C₃—C-cycloalkyl; and —C(═O)N(R^{b})₂ wherein R^{b} is as disclosed herein, preferably wherein R^{b} are independently selected from the group consisting of hydrogen, C₁-C₆-alkyl, —C₁-C₆-alkyl-C₃-C₈-cycloalkyl and aryl that may be substituted with one or more halogen atoms.

In the above formula (I), when n is 2, 3, 4 or 5, at least (n−1) of said R3 is/are independently selected from the group consisting of halogen and C₁-C₃-alkyl.

In the above formula (I), n is preferably 0, 1 or 2, more preferably 0 or 1.

In some embodiments, compounds of the present invention are compounds of formula (I)

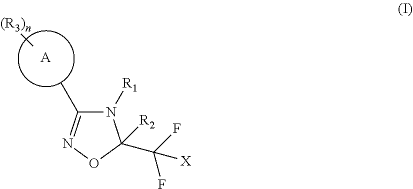

wherein
A is selected from the group consisting of 9- or 10-membered bicyclic heterocyclyl, C₆-C₁₀-aryl, 5- or 6-membered monocyclic heteroaryl and 9- or 10-membered bicyclic heteroaryl, preferably A is selected from the group consisting of 10-membered bicyclic heterocycle comprising one nitrogen atom (e.g. dihydroisoquinolinyl, tetrahydroquinolinyl, tetrahydroisoquinolinyl), C₆-C₁₀-aryl (e.g. phenyl or naphthyl), 5- or 6-membered monocyclic heteroaryl comprising one or two nitrogen atoms (e.g. pyridinyl, pyrimidinyl) and 9- or 10-membered bicyclic heteroaryl comprising one or two nitrogen atom (e.g. indolyl, benzimidazolyl, indazolyl);
R1 is selected from the group consisting of hydrogen, C₁-C₆-alkyl and —C(═O)R^{a} wherein R^{a} is as described herein, preferably wherein R^{a} is C₁-C₆-alkyl,
   preferably is hydrogen or —C(═O)R^{a} wherein R^{a} is as described herein, preferably wherein R^{a} is C₁-C₆-alkyl;
R2 is hydrogen or C₁-C₆-alkyl,
   preferably is hydrogen;
n is 0, 1 or 2,
   preferably is 0 or 1;
R3 is selected from the group consisting of hydroxy, halogen, oxo, C₁-C₆-alkyl, C₁-C₆-alkylsulfanyl, arylsulfanyl, arylsulfinyl, C₁-C₆-alkylsulfonyl, arylsulfonyl, carbocyclyl, heterocyclyl, aryl, heteroaryl, —NR^{b}C(═O)R^{b}, —OC(═O)R^{b}, —N(R^{b})₂, —NR^{b}C(═O)OR^{b}, —N═CR^{b}, —N(R^{b})₂, —OC(═O)N(R^{b})₂, —C(═NOH)R^{b}, —C(═NOH)N(R^{b})₂, —C(═O)R^{b}, —C(═O)(OR^{b}), —C(═O)N(R^{b})₂, —C(═O)NR^{b}N(R^{b})₂, —C(═S)N(R^{b})₂, —C₁-C₆-(halo)alkyl-O—C₁-C₆-(halo)alkyl, —C₁-C₆-(halo)alkyl-NR^{b}C(═O)OR^{b}, —C₁-C₆-(halo)alkyl-N(OR^{b})C(═O)OR^{b}, —C₁-C₆-(halo)alkyl-NR^{b}C(═O)R^{b}, —C₁-C₆-(halo)alkyl-N(OR^{b})C(═O)R^{b}, —C₁-C₆-(halo)alkyl-aryl, —C₁-C₆-(halo)alkyl-C(═O)N(R^{b})₂, —C₁-C₆-alkyl-R wherein the C₁-C₆-alkyl in said C₁-C₆-alkyl-R^{c} is substituted with two substituents on a same carbon atom that form together with the carbon atom to which they are attached a carbocyclyl (e.g. a cyclopropyl), wherein any of the previously cited carbocyclyl, heterocyclyl, aryl and heteroaryl groups may be substituted with one or more R³¹ substituents as described herein (preferably R³¹ is independently selected from the group consisting halogen, oxo, C₁-C₆-alkyl, C₁-C₆-alkoxy, heteroaryl, —NR³¹¹C(═O)OR³¹¹ and —C(═O)(OR³¹¹) with R³¹¹ being hydrogen or C₁-C₆-alkyl), and wherein R^{b} and R^{c} are as described herein, preferably wherein R^{b} is selected from the group consisting of hydrogen, C₁-C₆-alkyl, C₂-C₆-alkynyl, C₁-C₆-alkylsulfanyl, carbocyclyl, aryl, —C₁-C₆-alkyl-carbocyclyl, —C₁-C₆-alkyl-O-aryl and —C₁-C₆-alkyl-C₁-C₆-alkoxy (wherein any of the previously cited carbocyclyl or aryl, group may be substituted by one or more R^{b1} substituents as described herein, preferably wherein R^{b1} is halogen or C₁-C₆-alkyl), preferably R^{c} is —NR^{b}C(═O)R^{b} with R^{b} being a C₁-C₆-alkyl.

In some embodiments, compounds of the present invention are compounds of formula (I)

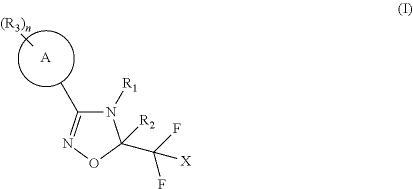

wherein
A is selected from the group consisting of 9- or 10-membered bicyclic heterocyclyl, C₆-C₁₀-aryl, 5- or 6-membered monocyclic heteroaryl and 9- or 10-membered bicyclic heteroaryl, preferably A is selected from the group consisting of 10-membered bicyclic heterocycle comprising one nitrogen atom (e.g. dihydroisoquinolinyl, tetrahydroquinolinyl, tetrahydroisoquinolinyl), C₆-C₁₀-aryl (e.g. phenyl or naphthyl), 5- or 6-membered monocyclic heteroaryl comprising one or two nitrogen atoms (e.g. pyridinyl, pyrimidinyl) and 9- or 10-membered bicyclic heteroaryl comprising one or two nitrogen atom (e.g. indolyl, benzimidazolyl, indazolyl);
R1 is selected from the group consisting of hydrogen, C₁-C₆-alkyl and —C(═O)R^{a} wherein R^{a} is as described herein, preferably wherein R^{a} is C₁-C₆-alkyl;
R2 is hydrogen or C₁-C₆-alkyl;
n is 0 or 1;
R3 is selected from the group consisting of hydroxy, oxo, C₁-C₆-alkyl, C₁-C₆-alkylsulfanyl, arylsulfanyl, arylsulfinyl, C₁-C₆-alkylsulfonyl, arylsulfonyl, carbocyclyl, heterocyclyl, aryl, heteroaryl, —OC(═O)R^{b}, —N(R^{b})₂, —NR^{b}C(═O)OR^{b}, —OC(═O)N(R^{b})₂, —C(═NOH)R^{b}, —C(═NOH)N(R^{b})₂, —C(═O)R^{b}, —C(═O)(OR^{b}), —C(═O)N(R^{b})₂, —C(═O)NR^{b}N(R^{b})₂, —C(═S)N(R^{b})₂, —C₁-C₆-(halo)alkyl-NR^{b}C(═O)OR^{b}, —C₁-C₆-(halo)alkyl-N(OR^{b})C(═O)OR^{b}, —C₁-C₆-(halo)alkyl-NR^{b}C(═O)R^{b}, —C₁-C₆-(halo)alkyl-N(OR^{b})C(═O)R^{b} and —C₁-C₆-(halo)alkyl-aryl,
   wherein any carbocyclyl, heterocyclyl, aryl and heteroaryl group may be substituted with one or more R³¹ substituents as described herein, preferably R³¹ is selected from the group consisting of halogen, oxo and C₁-C₆-alkyl,
   wherein R^{b} is as described herein, preferably wherein R^{b} is selected from the group consisting of hydrogen, C₁-C₆-alkyl, C₁-C₆-alkoxy, C₁-C₆-alkylsulfanyl, carbocyclyl, aryl, heterocyclyl, heteroaryl and —C₁-C₆-alkyl-carbocyclyl, wherein any carbocyclyl, aryl, heterocyclyl and heteroaryl group may be substituted by one or more R^{b1} substituents as described herein.

The present invention also relates to any compounds of formula (I) disclosed in Table 1.

The compounds of formula (I) according to the present invention may be used as fungicides (i.e. for controlling phytopathogenic fungi, in particular fungi causing rust diseases, or Oomyctes in crop protection).

### Compositions and Formulations

The present invention further relates to a composition, in particular a composition for controlling unwanted microorganisms, comprising one or more compounds of formula (I). The composition is preferably is a fungicidal composition.

The composition typically comprises one or more compounds of formula (I) and one or more acceptable carriers, in particular one or more agriculturally acceptable carriers.

A carrier is a solid or liquid, natural or synthetic, organic or inorganic substance that is generally inert. The carrier generally improves the application of the compounds, for instance, to plants, plants parts or seeds. Examples of suitable solid carriers include, but are not limited to, ammonium salts, natural rock flours, such as kaolins, clays, talc, chalk, quartz, attapulgite, montmorillonite and diatomaceous earth, and synthetic rock flours, such as finely divided silica, alumina and silicates. Examples of typically useful solid carriers for preparing granules include, but are not limited to crushed and fractionated natural rocks such as calcite, marble, pumice, sepiolite and dolomite, synthetic granules of inorganic and organic flours and granules of organic material such as paper, sawdust, coconut shells, maize cobs and tobacco stalks. Examples of suitable liquid carriers include, but are not limited to, water, organic solvents and combinations thereof. Examples of suitable solvents include polar and nonpolar organic chemical liquids, for example from the classes of aromatic and nonaromatic hydrocarbons (such as cyclohexane, paraffins, alkylbenzenes, xylene, toluene alkylnaphthalenes, chlorinated aromatics or chlorinated aliphatic hydrocarbons such as chlorobenzenes, chloroethylenes or methylene chloride), alcohols and polyols (which may optionally also be substituted, etherified and/or esterified, such as butanol or glycol), ketones (such as acetone, methyl ethyl ketone, methyl isobutyl ketone or cyclohexanone), esters (including fats and oils) and (poly)ethers, unsubstituted and substituted amines, amides (such as dimethylformamide), lactams (such as N-alkylpyrrolidones) and lactones, sulphones and sulphoxides (such as dimethyl sulphoxide). The carrier may also be a liquefied gaseous extender, i.e. liquid which is gaseous at standard temperature and under standard pressure, for example aerosol propellants such as halohydrocarbons, butane, propane, nitrogen and carbon dioxide. The amount of carrier typically ranges from 1 to 99.99%, preferably from 5 to 99.9%, more preferably from 10 to 99.5%, and most preferably from 20 to 99% by weight of the composition.

The composition may further comprise one or more acceptable auxiliaries which are customary for formulating compositions (e.g. agrochemical compositions), such as one or more surfactants.

The surfactant can be an ionic (cationic or anionic) or non-ionic surfactant, such as ionic or non-ionic emulsifier(s), foam former(s), dispersant(s), wetting agent(s) and any mixtures thereof. Examples of suitable surfactants include, but are not limited to, salts of polyacrylic acid, salts of lignosulphonic acid, salts of phenolsulphonic acid or naphthalenesulphonic acid, polycondensates of ethylene and/or propylene oxide with fatty alcohols, fatty acids or fatty amines (polyoxyethylene fatty acid esters, polyoxyethylene fatty alcohol ethers, for example alkylaryl polyglycol ethers), substituted phenols (preferably alkylphenols or arylphenols), salts of sulphosuccinic esters, taurine derivatives (preferably alkyl taurates), phosphoric esters of polyethoxylated alcohols or phenols, fatty esters of polyols and derivatives of compounds containing sulphates, sulphonates, phosphates (for example, alkylsulphonates, alkyl sulphates, arylsulphonates) and protein hydrolysates, lignosulphite waste liquors and methylcellulose. A surfactant is typically used when the compound of the formula (I) and/or the carrier is insoluble in water and the application is made with water. Then, the amount of surfactants typically ranges from 5 to 40% by weight of the composition.

Further examples of auxiliaries which are customary for formulating agrochemical compositions include water repellents, siccatives, binders (adhesive, tackifier, fixing agent, such as carboxymethycellulose, natural and synthetic polymers in the form of powders, granules or latices, such as gum arabic, polyvinyl alcohol and polyvinyl acetate, natural phospholipids such as cephalins and lecithins and synthetic phospholipids, polyvinylpyrrolidone, polyvinyl acetate, polyvinyl alcohol and tylose), thickeners, stabilizers (e.g. cold stabilizers, preservatives, antioxidants, light stabilizers, or other agents which improve chemical and/or physical stability), dyes or pigments (such as inorganic pigments, e.g. iron oxide, titanium oxide and Prussian Blue; organic dyes, e.g. alizarin, azo and metal phthalocyanine dyes), antifoams (e.g. silicone antifoams and magnesium stearate), preservatives (e.g. dichlorophene and benzyl alcohol hemiformal), secondary thickeners (cellulose derivatives, acrylic acid derivatives, xanthan, modified clays and finely divided silica), stickers, gibberellins and processing auxiliaries, mineral and vegetable oils, perfumes, waxes, nutrients (including trace nutrients, such as salts of iron, manganese, boron, copper, cobalt, molybdenum and zinc), protective colloids, thixotropic substances, penetrants, sequestering agents and complex formers.

The choice of the auxiliaries is related to the intended mode of application of the compound of the formula (I) and/or on the physical properties. Furthermore, the auxiliaries may be chosen to impart particular properties (technical, physical and/or biological properties) to the compositions or use forms prepared therefrom. The choice of auxiliaries may allow customizing the compositions to specific needs.

The composition of the invention may be in any customary form, such as solutions (e.g aqueous solutions), emulsions, wettable powders, water- and oil-based suspensions, powders, dusts, pastes, soluble powders, soluble granules, granules for broadcasting, suspoemulsion concentrates, natural or synthetic products impregnated with the compound of the invention, fertilizers and also microencapsulations in polymeric substances. The compound of the invention may be present in a suspended, emulsified or dissolved form.

The composition of the invention may be provided to the end user as ready-for-use formulation, i.e. the compositions may be directly applied to the plants or seeds by a suitable device, such as a spraying or dusting device. Alternatively, the compositions may be provided to the end user in the form of concentrates which have to be diluted, preferably with water, prior to use.

The composition of the invention can be prepared in conventional manners, for example by mixing the compound of the invention with one or more suitable auxiliaries, such as disclosed herein above.

The composition according to the invention contains generally from 0.01 to 99% by weight, from 0.05 to 98% by weight, preferably from 0.1 to 95% by weight, more preferably from 0.5 to 90% by weight, most preferably from 1 to 80% by weight of the compound of the invention.

The compound and the composition of the invention can be mixed with other active ingredients like fungicides, bactericides, acaricides, nematicides, insecticides, herbicides, fertilizers, growth regulators, safeners or semiochemicals. This may allow to broaden the activity spectrum or to prevent development of resistance. Examples of known fungicides, insecticides, acaricides, nematicides and bactericides are disclosed in the Pesticide Manual, 17th Edition.

Examples of especially preferred fungicides which could be mixed with the compound and the composition of the invention are:

1) Inhibitors of the ergosterol biosynthesis, for example (1.001) cyproconazole, (1.002) difenoconazole, (1.003) epoxiconazole, (1.004) fenhexamid, (1.005) fenpropidin, (1.006) fenpropimorph, (1.007) fenpyrazamine, (1.008) fluquinconazole, (1.009) flutriafol, (1.010) imazalil, (1.011) imazalil sulfate, (1.012) ipconazole, (1.013) metconazole, (1.014) myclobutanil, (1.015) paclobutrazol, (1.016) prochloraz, (1.017) propiconazole, (1.018) prothioconazole, (1.019) Pyrisoxazole, (1.020) spiroxamine, (1.021) tebuconazole, (1.022) tetraconazole, (1.023) triadimenol, (1.024) tridemorph, (1.025) triticonazole, (1.026) (1R,2S,5S)-5-(4-chlorobenzyl)-2-(chloromethyl)-2-methyl-1-(1H-1,2,4-triazol-1-ylmethyl)cyclopentanol, (1.027) (1S,2R,5R)-5-(4-chlorobenzyl)-2-(chloromethyl)-2-methyl-1-(1H-1,2,4-triazol-1-ylmethyl)cyclopentanol, (1.028) (2R)-2-(1-chlorocyclopropyl)-4-[(1R)-2,2-dichlorocyclopropyl]-1-(1H-1,2,4-triazol-1-yl)butan-2-ol, (1.029) (2R)-2-(1-chlorocyclopropyl)-4-[(1S)-2,2-dichlorocyclopropyl]-1-(1H-1,2,4-triazol-1-yl)butan-2-ol, (1.030) (2R)-2-[4-(4-chlorophenoxy)-2-(trifluoromethyl)phenyl]-1-(1H-1,2,4-triazol-1-yl)propan-2-ol, (1.031) (2S)-2-(1-chlorocyclopropyl)-4-[(1R)-2,2-dichlorocyclopropyl]-1-(1H-1,2,4-triazol-1-yl)butan-2-ol, (1.032) (2S)-2-(1-chlorocyclopropyl)-4-[(1S)-2,2-dichlorocyclopropyl]-1-(1H-1,2,4-triazol-1-yl)butan-2-ol, (1.033) (2S)-2-[4-(4-chlorophenoxy)-2-(trifluoromethyl)phenyl]-1-(1H-1,2,4-triazol-1-yl)propan-2-ol, (1.034) (R)-[3-(4-chloro-2-fluorophenyl)-5-(2,4-difluorophenyl)-1,2-oxazol-4-yl](pyridin-3-yl)methanol, (1.035) (S)-[3-(4-chloro-2-fluorophenyl)-5-(2,4-difluorophenyl)-1,2-oxazol-4-yl](pyridin-3-yl)methanol, (1.036) [3-(4-chloro-2-fluorophenyl)-5-(2,4-difluorophenyl)-1,2-oxazol-4-yl](pyridin-3-yl)methanol, (1.037) 1-({(2R,4S)-2-[2-chloro-4-(4-chlorophenoxy)phenyl]-4-methyl-1,3-dioxolan-2-yl}methyl)-1H-1,2,4-triazole, (1.038) 1-({(2S,4S)-2-[2-chloro-4-(4-chlorophenoxy)phenyl]-4-methyl-1,3-dioxolan-2-yl}methyl)-1H-1,2,4-triazole, (1.039) 1-{[3-(2-chlorophenyl)-2-(2,4-difluorophenyl)oxiran-2-yl]methyl}-1H-1,2,4-triazol-5-yl thiocyanate, (1.040) 1-{[rel(2R,3R)-3-(2-chlorophenyl)-2-(2,4-difluorophenyl)oxiran-2-yl]methyl}-1H-1,2,4-triazol-5-yl thiocyanate, (1.041) 1-{[rel(2R,3S)-3-(2-chlorophenyl)-2-(2,4-difluorophenyl)oxiran-2-yl]methyl}-1H-1,2,4-triazol-5-yl thiocyanate, (1.042) 2-[(2R,4R,5R)-1-(2,4-dichlorophenyl)-5-hydroxy-2,6,6-trimethylheptan-4-yl]-2,4-dihydro-3H-1,2,4-triazole-3-thione, (1.043) 2-[(2R,4R,5S)-1-(2,4-dichlorophenyl)-5-hydroxy-2,6,6-trimethylheptan-4-yl]-2,4-dihydro-3H-1,2,4-triazole-3-thione, (1.044) 2-[(2R,4S,5R)-1-(2,4-dichlorophenyl)-5-hydroxy-2,6,6-trimethylheptan-4-yl]-2,4-dihydro-3H-1,2,4-triazole-3-thione, (1.045) 2-[(2R,4S,5S)-1-(2,4-dichlorophenyl)-5-hydroxy-2,6,6-trimethylheptan-4-yl]-2,4-dihydro-3H-1,2,4-triazole-3-thione, (1.046) 2-[(2S,4R,5R)-1-(2,4-dichlorophenyl)-5-hydroxy-2,6,6-trimethylheptan-4-yl]-2,4-dihydro-3H-1,2,4-triazole-3-thione, (1.047) 2-[(2S,4R,5S)-1-(2,4-dichlorophenyl)-5-hydroxy-2,6,6-trimethylheptan-4-yl]-2,4-dihydro-3H-1,2,4-triazole-3-thione, (1.048) 2-[(2S,4S,5R)-1-(2,4-dichlorophenyl)-5-hydroxy-2,6,6-trimethylheptan-4-yl]-2,4-dihydro-3H-1,2,4-triazole-3-thione, (1.049) 2-[(2S,4S,5S)-1-(2,4-dichlorophenyl)-5-hydroxy-2,6,6-trimethylheptan-4-yl]-2,4-dihydro-3H-1,2,4-triazole-3-thione, (1.050) 2-[1-(2,4-dichlorophenyl)-5-hydroxy-2,6,6-trimethylheptan-4-yl]-2,4-dihydro-3H-1,2,4-triazole-3-thione, (1.051) 2-[2-chloro-4-(2,4-dichlorophenoxy)phenyl]-1-(1H-1,2,4-triazol-1-yl)propan-2-ol, (1.052) 2-[2-chloro-4-(4-chlorophenoxy)phenyl]-1-(1H-1,2,4-triazol-1-yl)butan-2-ol, (1.053) 2-[4-(4-chlorophenoxy)-2-(trifluoromethyl)phenyl]-1-(1H-1,2,4-triazol-1-yl)butan-2-ol, (1.054) 2-[4-(4-chlorophenoxy)-2-(trifluoromethyl)phenyl]-1-(1H-1,2,4-triazol-1-yl)pentan-2-ol, (1.055) 2-[4-(4-chlorophenoxy)-2-(trifluoromethyl)phenyl]-1-(1H-1,2,4-triazol-1-yl)propan-2-ol, (1.056) 2-{[3-(2-chlorophenyl)-2-(2,4-difluorophenyl)oxiran-2-yl]methyl}-2,4-dihydro-3H-1,2,4-triazole-3-thione, (1.057) 2-{[rel(2R,3R)-3-(2-chlorophenyl)-2-(2,4-difluorophenyl)oxiran-2-yl]methyl}-2,4-dihydro-3H-1,2,4-triazole-3-thione, (1.058) 2-{[rel(2R,3S)-3-(2-chlorophenyl)-2-(2,4-difluorophenyl)oxiran-2-yl]methyl}-2,4-dihydro-3H-1,2,4-triazole-3-thione, (1.059) 5-(4-chlorobenzyl)-2-(chloromethyl)-2-methyl-1-(1H-1,2,4-triazol-1-ylmethyl)cyclopentanol, (1.060) 5-(allylsulfanyl)-1-{[3-(2-chlorophenyl)-2-(2,4-difluorophenyl)oxiran-2-yl]methyl}-1H-1,2,4-triazole, (1.061) 5-(allylsulfanyl)-1-{[rel(2R,3R)-3-(2-chlorophenyl)-2-(2,4-difluorophenyl)oxiran-2-yl]methyl}-1H-1,2,4-triazole, (1.062) 5-(allylsulfanyl)-1-{[rel(2R,3S)-3-(2-chlorophenyl)-2-(2,4-difluorophenyl)oxiran-2-yl]methyl}-1H-1,2,4-triazole, (1.063) N′-(2,5-dimethyl-4-{[3-(1,1,2,2-tetrafluoroethoxy)phenyl]sulfanyl}phenyl)-N-ethyl-N-methylimidoformamide, (1.064) N′-(2,5-dimethyl-4-{[3-(2,2,2-trifluoroethoxy)phenyl]sulfanyl}phenyl)-N-ethyl-N-methylimidoformamide, (1.065) N′-(2,5-dimethyl-4-{[3-(2,2,3,3-tetrafluoropropoxy)phenyl]sulfanyl}phenyl)-N-ethyl-N-methylimidoformamide, (1.066) N′-(2,5-dimethyl-4-{[3-(pentafluoroethoxy)phenyl]sulfanyl}phenyl)-N-ethyl-N-methylimidoformamide, (1.067) N′-(2,5-dimethyl-4-{3-[(1,1,2,2-tetrafluoroethyl)sulfanyl]phenoxy}phenyl)-N-ethyl-N-methylimidoformamide, (1.068) N′-(2,5-dimethyl-4-{3-[(2,2,2-trifluoroethyl)sulfanyl]phenoxy}phenyl)-N-ethyl-N-methylimidoformamide, (1.069) N′-(2,5-dimethyl-4-{3-[(2,2,3,3-tetrafluoropropyl)sulfanyl]phenoxy}phenyl)-N-ethyl-N-methylimidoformamide, (1.070) N′-(2,5-dimethyl-4-{3-[(pentafluoroethyl)sulfanyl]phenoxy}phenyl)-N-ethyl-N-methylimidoformamide, (1.071) N′-(2,5-dimethyl-4-phenoxyphenyl)-N-ethyl-N-methylimidoformamide, (1.072) N′-(4-{[3-(difluoromethoxy)phenyl]sulfanyl}-2,5-dimethylphenyl)-N-ethyl-N-methylimidoformamide, (1.073) N′-(4-{3-[(difluoromethyl)sulfanyl]phenoxy}-2,5-dimethylphenyl)-N-ethyl-N-methylimidoformamide, (1.074) N′-[5-bromo-6-(2,3-dihydro-1H-inden-2-yloxy)-2-methylpyridin-3-yl]-N-ethyl-N-methylimidoformamide, (1.075) N′-{4-[(4,5-dichloro-1,3-thiazol-2-yl)oxy]-2,5-dimethylphenyl}-N-ethyl-N-methylimidoformamide, (1.076) N′-{5-bromo-6-[(1R)-1-(3,5-difluorophenyl)-ethoxy]-2-methylpyridin-3-yl}-N-ethyl-N-methylimidoformamide, (1.077) N′-{5-bromo-6-[(1S)-1-(3,5-difluorophenyl)ethoxy]-2-methylpyridin-3-yl}-N-ethyl-N-methylimidoformamide, (1.078) N′-{5-bromo-6-[(cis-4-isopropylcyclohexyl)oxy]-2-methylpyridin-3-yl}-N-ethyl-N-methylimidoformamide, (1.079) N′-{5-bromo-6-[(trans-4-isopropylcyclohexyl)oxy]-2-methylpyridin-3-yl}-N-ethyl-N-methylimidoformamide, (1.080) N′-{5-bromo-6-[1-(3,5-difluorophenyl)ethoxy]-2-methylpyridin-3-yl}-N-ethyl-N-methylimidoformamide, (1.081) Mefentrifluconazole, (1.082) Ipfentrifluconazole, (1.083) 2-[6-(4-bromophenoxy)-2-(trifluoromethyl)-3-pyridyl]-1-(1,2,4-triazol-1-yl)propan-2-ol, (1.084) 2-[6-(4-chlorophenoxy)-2-(trifluoromethyl)-3-pyridyl]-1-(1,2,4-triazol-1-yl)propan-2-ol, (1.085) 3-[2-(1-chlorocyclopropyl)-3-(3-chloro-2-fluoro-phenyl)-2-hydroxy-propyl]imidazole-4-carbonitrile, (1.086) 4-[[6-[rac-(2R)-2-(2,4-difluorophenyl)-1,1-difluoro-2-hydroxy-3-(5-thioxo-4H-1,2,4-triazol-1-yl)propyl]-3-pyridyl]oxy]benzonitrile.
2) Inhibitors of the respiratory chain at complex I or II, for example (2.001) benzovindiflupyr, (2.002) bixafen, (2.003) boscalid, (2.004) carboxin, (2.005) fluopyram, (2.006) flutolanil, (2.007) fluxapyroxad, (2.008) furametpyr, (2.009) Isofetamid, (2.010) isopyrazam (anti-epimeric enantiomer 1R,4S,9S), (2.011) isopyrazam (anti-epimeric enantiomer 1S,4R,9R), (2.012) isopyrazam (anti-epimeric racemate 1RS,4SR,9SR), (2.013) isopyrazam (mixture of syn-epimeric racemate 1RS,4SR,9RS and anti-epimeric racemate 1RS,4SR,9SR), (2.014) isopyrazam (syn-epimeric enantiomer 1R,4S,9R), (2.015) isopyrazam (syn-epimeric enantiomer 1S,4R,9S), (2.016) isopyrazam (syn-epimeric racemate 1RS,4SR,9RS), (2.017) penflufen, (2.018) penthiopyrad, (2.019) pydiflumetofen, (2.020) Pyraziflumid, (2.021) sedaxane, (2.022) 1,3-dimethyl-N-(1,1,3-trimethyl-2,3-dihydro-1H-inden-4-yl)-1H-pyrazole-4-carboxamide, (2.023) 1,3-dimethyl-N-[(3R)-1,1,3-trimethyl-2,3-dihydro-1H-inden-4-yl]-1H-pyrazole-4-carboxamide, (2.024) 1,3-dimethyl-N-[(3S)-1,1,3-trimethyl-2,3-dihydro-1H-inden-4-yl]-1H-pyrazole-4-carboxamide, (2.025) 1-methyl-3-(trifluoromethyl)-N-[2′-(trifluoromethyl)biphenyl-2-yl]-1H-pyrazole-4-carboxamide, (2.026) 2-fluoro-6-(trifluoromethyl)-N-(1,1,3-trimethyl-2,3-dihydro-1H-inden-4-yl)benzamide, (2.027) 3-(difluoromethyl)-1-methyl-N-(1,1,3-trimethyl-2,3-dihydro-1H-inden-4-yl)-1H-pyrazole-4-carboxamide, (2.028) 3-(difluoromethyl)-1-methyl-N-[(3R)-1,1,3-trimethyl-2,3-dihydro-1H-inden-4-yl]-1H-pyrazole-4-carboxamide, (2.029) 3-(difluoromethyl)-1-methyl-N-[(3S)-1,1,3-trimethyl-2,3-dihydro-1H-inden-4-yl]-1H-pyrazole-4-carboxamide, (2.030) 3-(difluoromethyl)-N-(7-fluoro-1,1,3-trimethyl-2,3-dihydro-1H-inden-4-yl)-1-methyl-1H-pyrazole-4-carboxamide, (2.031) 3-(difluoromethyl)-N-[(3R)-7-fluoro-1,1,3-trimethyl-2,3-dihydro-1H-inden-4-yl]-1-methyl-1H-pyrazole-4-carboxamide, (2.032) 3-(difluoromethyl)-N-[(3S)-7-fluoro-1,1,3-trimethyl-2,3-dihydro-1H-inden-4-yl]-1-methyl-1H-pyrazole-4-carboxamide, (2.033) 5,8-difluoro-N-[2-(2-fluoro-4-{[4-(trifluoromethyl)pyridin-2-yl]oxy}phenyl)ethyl]quinazolin-4-amine, (2.034) N-(2-cyclopentyl-5-fluorobenzyl)-N-cyclopropyl-3-(difluoromethyl)-5-fluoro-1-methyl-1H-pyrazole-4-carboxamide, (2.035) N-(2-tert-butyl-5-methylbenzyl)-N-cyclopropyl-3-(difluoromethyl)-5-fluoro-1-methyl-1H-pyrazole-4-carboxamide, (2.036) N-(2-tert-butylbenzyl)-N-cyclopropyl-3-(difluoromethyl)-5-fluoro-1-methyl-1H-pyrazole-4-carboxamide, (2.037) N-(5-chloro-2-ethylbenzyl)-N-cyclopropyl-3-(difluoromethyl)-5-fluoro-1-methyl-1H-pyrazole-4-carboxamide, (2.038) N-(5-chloro-2-isopropylbenzyl)-N-cyclopropyl-3-(difluoromethyl)-5-fluoro-1-methyl-1H-pyrazole-4-carboxamide, (2.039) N-[(1R,4S)-9-(dichloromethylene)-1,2,3,4-tetrahydro-1,4-methanonaphthalen-5-yl]-3-(difluoromethyl)-1-methyl-1H-pyrazole-4-carboxamide, (2.040) N—[(S,4R)-9-(dichloromethylene)-1,2,3,4-tetrahydro-1,4-methanonaphthalen-5-yl]-3-(difluoromethyl)-1-methyl-1H-pyrazole-4-carboxamide, (2.041) N-[1-(2,4-dichlorophenyl)-1-methoxypropan-2-yl]-3-(difluoromethyl)-1-methyl-1H-pyrazole-4-carboxamide, (2.042) N-[2-chloro-6-(trifluoromethyl)benzyl]-N-cyclopropyl-3-(difluoromethyl)-5-fluoro-1-methyl-1H-pyrazole-4-carboxamide, (2.043) N-[3-chloro-2-fluoro-6-(trifluoromethyl)benzyl]-N-cyclopropyl-3-(difluoromethyl)-5-fluoro-1-methyl-1H-pyrazole-4-carboxamide, (2.044) N-[5-chloro-2-(trifluoromethyl)benzyl]-N-cyclopropyl-3-(difluoromethyl)-5-fluoro-1-methyl-1H-pyrazole-4-carboxamide, (2.045) N-cyclopropyl-3-(difluoromethyl)-5-fluoro-1-methyl-N-[5-methyl-2-(trifluoromethyl)benzyl]-1H-pyrazole-4-carboxamide, (2.046) N-cyclopropyl-3-(difluoromethyl)-5-fluoro-N-(2-fluoro-6-isopropylbenzyl)-1-methyl-1H-pyrazole-4-carboxamide, (2.047) N-cyclopropyl-3-(difluoromethyl)-5-fluoro-N-(2-isopropyl-5-methylbenzyl)-1-methyl-1H-pyrazole-4-carboxamide, (2.048) N-cyclopropyl-3-(difluoromethyl)-5-fluoro-N-(2-isopropylbenzyl)-1-methyl-1H-pyrazole-4-carbothioamide, (2.049) N-cyclopropyl-3-(difluoromethyl)-5-fluoro-N-(2-isopropylbenzyl)-1-methyl-1H-pyrazole-4-carboxamide, (2.050) N-cyclopropyl-3-(difluoromethyl)-5-fluoro-N-(5-fluoro-2-isopropylbenzyl)-1-methyl-1H-pyrazole-4-carboxamide, (2.051) N-cyclopropyl-3-(difluoromethyl)-N-(2-ethyl-4,5-dimethylbenzyl)-5-fluoro-1-methyl-1H-pyrazole-4-carboxamide, (2.052) N-cyclopropyl-3-(difluoromethyl)-N-(2-ethyl-5-fluorobenzyl)-5-fluoro-1-methyl-1H-pyrazole-4-carboxamide, (2.053) N-cyclopropyl-3-(difluoromethyl)-N-(2-ethyl-5-methylbenzyl)-5-fluoro-1-methyl-1H-pyrazole-4-carboxamide, (2.054) N-cyclopropyl-N-(2-cyclopropyl-5-fluorobenzyl)-3-(difluoromethyl)-5-fluoro-1-methyl-1H-pyrazole-4-carboxamide, (2.055) N-cyclopropyl-N-(2-cyclopropyl-5-methylbenzyl)-3-(difluoromethyl)-5-fluoro-1-methyl-1H-pyrazole-4-carboxamide, (2.056) N-cyclopropyl-N-(2-cyclopropylbenzyl)-3-(difluoromethyl)-5-fluoro-1-methyl-1H-pyrazole-4-carboxamide.
3) Inhibitors of the respiratory chain at complex III, for example (3.001) ametoctradin, (3.002) amisulbrom, (3.003) azoxystrobin, (3.004) coumethoxystrobin, (3.005) coumoxystrobin, (3.006) cyazofamid, (3.007) dimoxystrobin, (3.008) enoxastrobin, (3.009) famoxadone, (3.010) fenamidone, (3.011) flufenoxystrobin, (3.012) fluoxastrobin, (3.013) kresoxim-methyl, (3.014) metominostrobin, (3.015) orysastrobin, (3.016) picoxystrobin, (3.017) pyraclostrobin, (3.018) pyrametostrobin, (3.019) pyraoxystrobin, (3.020) trifloxystrobin, (3.021) (2E)-2-{2-[({[(1E)-1-(3-{[(E)-1-fluoro-2-phenylvinyl]oxy}phenyl)ethylidene]amino}oxy)methyl]phenyl}-2-(methoxyimino)-N-methylacetamide, (3.022) (2E,3Z)-5-{[1-(4-chlorophenyl)-1H-pyrazol-3-yl]oxy}-2-(methoxyimino)-N,3-dimethylpent-3-enamide, (3.023) (2R)-2-{2-[(2,5-dimethylphenoxy)methyl]phenyl}-2-methoxy-N-methylacetamide, (3.024) (2S)-2-{2-[(2,5-dimethylphenoxy)methyl]phenyl}-2-methoxy-N-methylacetamide, (3.025) (3S,6S,7R,8R)-8-benzyl-3-[({3-[(isobutyryloxy)methoxy]-4-methoxypyridin-2-yl}carbonyl)amino]-6-methyl-4,9-dioxo-1,5-dioxonan-7-yl 2-methylpropanoate, (3.026) 2-{2-[(2,5-dimethylphenoxy)-methyl]phenyl}-2-methoxy-N-methylacetamide, (3.027) N-(3-ethyl-3,5,5-trimethylcyclohexyl)-3-formamido-2-hydroxybenzamide, (3.028) (2E,3Z)-5-{[1-(4-chloro-2-fluorophenyl)-1H-pyrazol-3-yl]oxy}-2-(methoxyimino)-N,3-dimethylpent-3-enamide, (3.029) methyl {5-[3-(2,4-dimethylphenyl)-1H-pyrazol-1-yl]-2-methylbenzyl}carbamate, (3.030) (1S)-2,2-bis(4-fluorophenyl)-1-methylethyl N-{[3-(acetyloxy)-4-methoxy-2-pyridyl]carbonyl}-L-alaninate.
4) Inhibitors of the mitosis and cell division, for example (4.001) carbendazim, (4.002) diethofencarb, (4.003) ethaboxam, (4.004) fluopicolide, (4.005) pencycuron, (4.006) thiabendazole, (4.007) thiophanate-methyl, (4.008) zoxamide, (4.009) 3-chloro-4-(2,6-difluorophenyl)-6-methyl-5-phenylpyridazine, (4.010) 3-chloro-5-(4-chlorophenyl)-4-(2,6-difluorophenyl)-6-methylpyridazine, (4.011) 3-chloro-5-(6-chloropyridin-3-yl)-6-methyl-4-(2,4,6-trifluorophenyl)pyridazine, (4.012) 4-(2-bromo-4-fluorophenyl)-N-(2,6-difluorophenyl)-1,3-dimethyl-1H-pyrazol-5-amine, (4.013) 4-(2-bromo-4-fluorophenyl)-N-(2-bromo-6-fluorophenyl)-1,3-dimethyl-1H-pyrazol-5-amine, (4.014) 4-(2-bromo-4-fluorophenyl)-N-(2-bromophenyl)-1,3-dimethyl-1H-pyrazol-5-amine, (4.015) 4-(2-bromo-4-fluoro-phenyl)-N-(2-chloro-6-fluorophenyl)-1,3-dimethyl-1H-pyrazol-5-amine, (4.016) 4-(2-bromo-4-fluoro-phenyl)-N-(2-chlorophenyl)-1,3-dimethyl-1H-pyrazol-5-amine, (4.017) 4-(2-bromo-4-fluorophenyl)-N-(2-fluorophenyl)-1,3-dimethyl-1H-pyrazol-5-amine, (4.018) 4-(2-chloro-4-fluorophenyl)-N-(2,6-difluoro-phenyl)-1,3-dimethyl-1H-pyrazol-5-amine, (4.019) 4-(2-chloro-4-fluorophenyl)-N-(2-chloro-6-fluoro-phenyl)-1,3-dimethyl-1H-pyrazol-5-amine, (4.020) 4-(2-chloro-4-fluorophenyl)-N-(2-chlorophenyl)-1,3-dimethyl-1H-pyrazol-5-amine, (4.021) 4-(2-chloro-4-fluorophenyl)-N-(2-fluorophenyl)-1,3-dimethyl-1H-pyrazol-5-amine, (4.022) 4-(4-chlorophenyl)-5-(2,6-difluorophenyl)-3,6-dimethylpyridazine, (4.023) N-(2-bromo-6-fluorophenyl)-4-(2-chloro-4-fluorophenyl)-1,3-dimethyl-1H-pyrazol-5-amine, (4.024) N-(2-bromophenyl)-4-(2-chloro-4-fluorophenyl)-1,3-dimethyl-1H-pyrazol-5-amine, (4.025) N-(4-chloro-2,6-difluorophenyl)-4-(2-chloro-4-fluorophenyl)-1,3-dimethyl-1H-pyrazol-5-amine.
5) Compounds capable to have a multisite action, for example (5.001) bordeaux mixture, (5.002) captafol, (5.003) captan, (5.004) chlorothalonil, (5.005) copper hydroxide, (5.006) copper naphthenate, (5.007) copper oxide, (5.008) copper oxychloride, (5.009) copper(2+) sulfate, (5.010) dithianon, (5.011) dodine, (5.012) folpet, (5.013) mancozeb, (5.014) maneb, (5.015) metiram, (5.016) metiram zinc, (5.017) oxine-copper, (5.018) propineb, (5.019) sulfur and sulfur preparations including calcium polysulfide, (5.020) thiram, (5.021) zineb, (5.022) ziram, (5.023) 6-ethyl-5,7-dioxo-6,7-dihydro-5H-pyrrolo[3′,4′:5,6][1,4]dithiino[2,3-c][1,2]thiazole-3-carbonitrile.
6) Compounds capable to induce a host defence, for example (6.001) acibenzolar-S-methyl, (6.002) isotianil, (6.003) probenazole, (6.004) tiadinil.
7) Inhibitors of the amino acid and/or protein biosynthesis, for example (7.001) cyprodinil, (7.002) kasugamycin, (7.003) kasugamycin hydrochloride hydrate, (7.004) oxytetracycline, (7.005) pyrimethanil, (7.006) 3-(5-fluoro-3,3,4,4-tetramethyl-3,4-dihydroisoquinolin-1-yl)quinoline. 8) Inhibitors of the ATP production, for example (8.001) silthiofam.
9) Inhibitors of the cell wall synthesis, for example (9.001) benthiavalicarb, (9.002) dimethomorph, (9.003) flumorph, (9.004) iprovalicarb, (9.005) mandipropamid, (9.006) pyrimorph, (9.007) valifenalate, (9.008) (2E)-3-(4-tert-butylphenyl)-3-(2-chloropyridin-4-yl)-1-(morpholin-4-yl)prop-2-en-1-one, (9.009) (2Z)-3-(4-tert-butylphenyl)-3-(2-chloropyridin-4-yl)-1-(morpholin-4-yl)prop-2-en-1-one.
10) Inhibitors of the lipid and membrane synthesis, for example (10.001) propamocarb, (10.002) propamocarb hydrochloride, (10.003) tolclofos-methyl.
11) Inhibitors of the melanin biosynthesis, for example (11.001) tricyclazole, (11.002) 2,2,2-trifluoroethyl {3-methyl-1-[(4-methylbenzoyl)amino]butan-2-yl}carbamate.
12) Inhibitors of the nucleic acid synthesis, for example (12.001) benalaxyl, (12.002) benalaxyl-M (kiralaxyl), (12.003) metalaxyl, (12.004) metalaxyl-M (mefenoxam).
13) Inhibitors of the signal transduction, for example (13.001) fludioxonil, (13.002) iprodione, (13.003) procymidone, (13.004) proquinazid, (13.005) quinoxyfen, (13.006) vinclozolin. 14) Compounds capable to act as an uncoupler, for example (14.001) fluazinam, (14.002) meptyldinocap.
15) Further compounds, for example (15.001) Abscisic acid, (15.002) benthiazole, (15.003) bethoxazin, (15.004) capsimycin, (15.005) carvone, (15.006) chinomethionat, (15.007) cufraneb, (15.008) cyflufenamid, (15.009) cymoxanil, (15.010) cyprosulfamide, (15.011) flutianil, (15.012) fosetyl-aluminium, (15.013) fosetyl-calcium, (15.014) fosetyl-sodium, (15.015) methyl isothiocyanate, (15.016) metrafenone, (15.017) mildiomycin, (15.018) natamycin, (15.019) nickel dimethyldithiocarbamate, (15.020) nitrothal-isopropyl, (15.021) oxamocarb, (15.022) Oxathiapiprolin, (15.023) oxyfenthiin, (15.024) pentachlorophenol and salts, (15.025) phosphorous acid and its salts, (15.026) propamocarb-fosetylate, (15.027) pyriofenone (chlazafenone), (15.028) tebufloquin, (15.029) tecloftalam, (15.030) tolnifanide, (15.031) 1-(4-{4-[(5R)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, (15.032) 1-(4-{4-[(5S)-5-(2,6-difluorophenyl)-4,5-dihydro-1,2-oxazol-3-yl]-1,3-thiazol-2-yl}piperidin-1-yl)-2-[5-methyl-3-(trifluoro-methyl)-1H-pyrazol-1-yl]ethanone, (15.033) 2-(6-benzylpyridin-2-yl)quinazoline, (15.034) 2,6-dimethyl-1H,5H-[1,4]dithiino[2,3-c:5,6-c′]dipyrrole-1,3,5,7(2H,6H)-tetrone, (15.035) 2-[3,5-bis(difluoromethyl)-1H-pyrazol-1-yl]-1-[4-(4-{5-[2-(prop-2-yn-1-yloxy)phenyl]-4,5-dihydro-1,2-oxazol-3-yl}-1,3-thiazol-2-yl)piperidin-1-yl]ethanone, (15.036) 2-[3,5-bis(difluoromethyl)-1H-pyrazol-1-yl]-1-[4-(4-{5-[2-chloro-6-(prop-2-yn-1-yloxy)phenyl]-4,5-dihydro-1,2-oxazol-3-yl}-1,3-thiazol-2-yl)piperidin-1-yl]ethanone, (15.037) 2-[3,5-bis(difluoromethyl)-1H-pyrazol-1-yl]-1-[4-(4-{5-[2-fluoro-6-(prop-2-yn-1-yloxy)phenyl]-4,5-dihydro-1,2-oxazol-3-yl}-1,3-thiazol-2-yl)piperidin-1-yl]ethanone, (15.038) 2-[6-(3-fluoro-4-methoxyphenyl)-5-methylpyridin-2-yl]quinazoline, (15.039) 2-{(5R)-3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}-3-chlorophenyl methanesulfonate, (15.040) 2-{(5S)-3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}-3-chlorophenyl methanesulfonate, (15.041) 2-{2-[(7,8-difluoro-2-methylquinolin-3-yl)oxy]-6-fluorophenyl}propan-2-ol, (15.042) 2-{2-fluoro-6-[(8-fluoro-2-methylquinolin-3-yl)oxy]phenyl}propan-2-ol, (15.043) 2-{3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}-3-chlorophenyl methanesulfonate, (15.044) 2-{3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}phenyl methanesulfonate, (15.045) 2-phenylphenol and salts, (15.046) 3-(4,4,5-trifluoro-3,3-dimethyl-3,4-dihydroisoquinolin-1-yl)quinoline, (15.047) 3-(4,4-difluoro-3,3-dimethyl-3,4-dihydroisoquinolin-1-yl)quinoline, (15.048) 4-amino-5-fluoropyrimidin-2-ol (tautomeric form: 4-amino-5-fluoropyrimidin-2(1H)-one), (15.049) 4-oxo-4-[(2-phenylethyl)amino]butanoic acid, (15.050) 5-amino-1,3,4-thiadiazole-2-thiol, (15.051) 5-chloro-N′-phenyl-N′-(prop-2-yn-1-yl)thiophene-2-sulfonohydrazide, (15.052) 5-fluoro-2-[(4-fluorobenzyl)oxy]pyrimidin-4-amine, (15.053) 5-fluoro-2-[(4-methylbenzyl)oxy]pyrimidin-4-amine, (15.054) 9-fluoro-2,2-dimethyl-5-(quinolin-3-yl)-2,3-dihydro-1,4-benzoxazepine, (15.055) but-3-yn-1-yl {6-[({[(Z)-(1-methyl-1H-tetrazol-5-yl)(phenyl)methylene]amino}oxy)methyl]pyridin-2-yl}carbamate, (15.056) ethyl (2Z)-3-amino-2-cyano-3-phenylacrylate, (15.057) phenazine-1-carboxylic acid, (15.058) propyl 3,4,5-trihydroxybenzoate, (15.059) quinolin-8-ol, (15.060) quinolin-8-ol sulfate (2:1), (15.061) tert-butyl {6-[({[(1-methyl-1H-tetrazol-5-yl)(phenyl)methylene]amino}oxy)methyl]pyridin-2-yl}carbamate, (15.062) 5-fluoro-4-imino-3-methyl-1-[(4-methylphenyl)sulfonyl]-3,4-dihydropyrimidin-2(1H)-one, (15.063) Metyltetraprole, (15.064) Aminopyrifen, (15.065) Pyrapropoyne, (15.066) (N′-[2-chloro-4-(2-fluorophenoxy)-5-methylphenyl]-N-ethyl-N-methylimidoformamide), (15.067) (N′-(2-chloro-5-methyl-4-phenoxyphenyl)-N-ethyl-N-methylimidoformamide), (15.068) (2-{2-[(7,8-difluoro-2-methylquinolin-3-yl)oxy]-6-fluorophenyl}propan-2-ol), (15.069) (5-bromo-1-(5,6-dimethylpyridin-3-yl)-3,3-dimethyl-3,4-dihydroisoquinoline), (15.070) (3-(4,4-difluoro-5,5-dimethyl-4,5-dihydrothieno[2,3-c]pyridin-7-yl)quinoline), (15.071) (1-(4,5-dimethyl-1H-benzimidazol-1-yl)-4,4-difluoro-3,3-dimethyl-3,4-dihydroisoquinoline), (15.072) 8-fluoro-3-(5-fluoro-3,3-dimethyl-3,4-dihydroisoquinolin-1-yl)quinolone, (15.073) 8-fluoro-3-(5-fluoro-3,3,4,4-tetramethyl-3,4-dihydroisoquinolin-1-yl)quinolone, (15.074) 3-(4,4-difluoro-3,3-dimethyl-3,4-dihydroisoquinolin-1-yl)-8-fluoroquinoline, (15.075) (N-methyl-N-phenyl-4-[5-(trifluoromethyl)-1,2,4-oxadiazol-3-yl]benzamide), (15.076) (methyl {4-[5-(trifluoromethyl)-1,2,4-oxadiazol-3-yl]phenyl}carbamate), (15.077) (N-{4-[5-(trifluoromethyl)-1,2,4-oxadiazol-3-yl]benzyl}cyclopropanecarboxamide), (15.078) N-methyl-4-(5-(trifluoromethyl)-1,2,4-oxadiazol-3-yl]benzamide, (15.079) N-[(E)-methoxyiminomethyl]-4-[5-(trifluoromethyl)-1,2,4-oxadiazol-3-yl]benzamide, (15.080) N-[(Z)-methoxyiminomethyl]-4-[5-(trifluoromethyl)-1,2,4-oxadiazol-3-yl]benzamide, (15.081) N-[4-[5-(trifluoromethyl)-1,2,4-oxadiazol-3-yl]phenyl]cyclopropanecarboxamide, (15.082) N-(2-fluorophenyl)-4-[5-(trifluoromethyl)-1,2,4-oxadiazol-3-yl]benzamide, (15.083) 2,2-difluoro-N-methyl-2-[4-[5-(trifluoromethyl)-1,2,4-oxadiazol-3-yl]phenyl]acetamide, (15.084) N-allyl-N-[[4-[5-(trifluoromethyl)-1,2,4-oxadiazol-3-yl)phenyl]methyl]acetamide, (15.085) N-[(E)-N-methoxy-C-methyl-carbonimidoyl]-4-(5-(trifluoromethyl)-1,2,4-oxadiazol-3-yl]benzamide, (15.086) N-[(Z)—N-methoxy-C-methyl-carbonimidoyl]-4-[5-(trifluoromethyl)-1,2,4-oxadiazol-3-yl]benzamide, (15.087) N-allyl-N-[[4-[5-(trifluoromethyl)-1,2,4-oxadiazol-3-yl]phenyl]methyl]propanamide, (15.088) 4,4-dimethyl-1-[[4-[5-(trifluoromethyl)-1,2,4-oxadiazol-3-yl]phenyl]methyl]pyrrolidin-2-one, (15.089) N-methyl-4-[5-(trifluoromethyl)-1,2,4-oxadiazol-3-yl]benzenecarbothioamide, (15.090) 5-methyl-1-[[4-[5-(trifluoromethyl)-1,2,4-oxadiazol-3-yl]phenyl]methyl]pyrrolidin-2-one, (15.091) N-((2,3-difluoro-4-[5-(trifluoromethyl)-1,2,4-oxadiazol-3-yl]phenyl]methyl]-3,3,3-trifluoro-propanamide, (15.092) 1-methoxy-1-methyl-3-[[4-[5-(trifluoromethyl}-1,2,4-oxadiazol-3-yl]phenyl]methyl]urea, (15.093) 1,1-diethyl-3-[[4-[5-(trifluoromethyl}-1,2,4-oxadiazol-3-yl]phenyl]methyl]urea, (15.094) N-[[4-[5-(trifluoromethyl)-1,2,4-oxadiazol-3-yl]phen-yl)methyl)propanamide, (15.095) N-methoxy-N-[[4-[5-(trifluoromethyl)-1,2,4-oxadiazol-3-yl]phenyl]methyl]cyclopropanecarboxamide, (15.096) 1-methoxy-3-methyl-1-[[4-[5-(trifluoromethyl)-1,2,4-oxadiazol-3-yl]phenyl]methyl]urea, (15.097) N-methoxy-N-[[4-[5-(trifluoromethyl)-1,2,4-oxadiazol-3-yl]phenyl]methyl)cyclopropanecarboxamide; (15.098) N,2-dimethoxy-N-[[4-[5-(trifluoromethyl}-1,2,4-oxadiazol-3-yl]phenyl]methyl]propanamide, (15.099) N-ethyl-2-methyl-N-[[4-[5-(trifluoromethyl)-1,2,4-oxadiazol-3-yl)phenyl]methyl]propanamide, (15.100) 1-methoxy-3-methyl-1-[[4-[5-(trifluoromethyl)-1,2,4-oxadiazol-3-yl]phenyl]methyl]urea, (15.101) 1,3-dimethoxy-1-[[4-[5-(trifluoromethyl)-1,2,4-oxadiazol-3-yl]phenyl]methyl]urea, (15.102) 3-ethyl-1-methoxy-1-[[4-[5-(trifluoromethyl)-1,2,4-oxadiazol-3-yl]phenyl]methyl]urea, (15.103) 1-[[4-[5-(t rifluoromethyl)-1,2,4-oxadiazol-3-yl]phenyl]methyl]piperidin-2-one, (15.104) 4,4-dimethyl-2-[[4-(5-(trifluoromethyl)-1,2,4-oxadiazol-3-yl]phenyl]methyl]isoxazolidin-3-one, (15.105) 5,5-dimethyl-2-[[4-[5-(trifluoromethyl)-1,2,4-oxadiazol-3-yl]phenyl]methyl]isoxazolidin-3-one, (15.106), 3,3-dimethyl-1-[[4-[5-(trifluoromethyl)-1,2,4-oxadiazol-3-yl]phenyl]methyl[piperidin-2-one, (15.107) 1-[[3-fluoro-4-(5-(trifluoromethyl)-1,2,4-oxadiazol-3-yl]phenyl]methyl]azepan-2-one, (15.108) 4,4-dimethyl-2-[[4-(5-(trifluoromethyl)-1,2,4-oxadiazol-3-yl]phenyl]methyl]isoxazolidin-3-one and (15.109) 5,5-dimethyl-2-[[4-[5-(trifluoromethyl)-1,2,4-oxadiazol-3-yl]phenyl]methyl]isoxazolidin-3-one, (15.110) ethyl (1-{4-[5-(trifluoromethyl)-1,2,4-oxadiazol-3-yl]benzyl}-1H-pyrazol-4-yl)acetate, (15.111) N,N-dimethyl-1-{4-[5-(trifluoromethyl)-1,2,4-oxadiazol-3-yl]benzyl}-1H-1,2,4-triazol-3-amine, (15.112) N-{2,3-difluoro-4-[5-(trifluoromethyl)-1,2,4-oxadiazol-3-yl]benzyl}butanamide.

All named mixing partners of the classes (1) to (15) as described here above can be present in the form of the free compound and/or, if their functional groups enable this, an agriculturally acceptable salt thereof.

The compounds of formula (I) and compositions comprising thereof may also be combined with one or more biological control agents.

Examples of biological control agents which may be combined with the compounds of formula (I) and compositions comprising thereof are:

(A) Antibacterial agents selected from the group of:
(A1) bacteria, such as (A1.1) Bacillus subtilis, in particular strain QST713/AQ713 (available as SERENADE OPTI or SERENADE ASO from Bayer CropScience LP, US, having NRRL Accession No. B21661 and described in U.S. Pat. No. 6,060,051); (A1.2) Bacillus amyloliquefaciens, in particular strain D747 (available as Double Nickel™ from Certis, US, having accession number FERM BP-8234 and disclosed in U.S. Pat. No. 7,094,592); (A1.3) Bacillus pumilus, in particular strain BU F-33 (having NRRL Accession No. 50185); (A1.4) Bacillus subtilis var. amyloliquefaciens strain FZB24 (available as Taegro® from Novozymes, US); (A1.5) a Paenibacillus sp. strain having Accession No. NRRL B-50972 or Accession No. NRRL B-67129 and described in International Patent Publication No. WO 2016/154297; and
(A2) fungi, such as (A2.1) Aureobasidium pullulans, in particular blastospores of strain DSM14940; (A2.2) Aureobasidium pullulans blastospores of strain DSM 14941; (A2.3) Aureobasidium pullulans, in particular mixtures of blastospores of strains DSM14940 and DSM14941;
(B) Fungicides selected from the group of:
(B1) bacteria, for example (B1.1) Bacillus subtilis, in particular strain QST713/AQ713 (available as SERENADE OPTI or SERENADE ASO from Bayer CropScience LP, US, having NRRL Accession No. B21661 and described in U.S. Pat. No. 6,060,051); (B1.2) Bacillus pumilus, in particular strain QST2808 (available as SONATA® from Bayer CropScience LP, US, having Accession No. NRRL B-30087 and described in U.S. Pat. No. 6,245,551); (B1.3) Bacillus pumilus, in particular strain GB34 (available as Yield Shield® from Bayer AG, DE); (B1.4) Bacillus pumilus, in particular strain BU F-33 (having NRRL Accession No. 50185); (B1.5) Bacillus amyloliquefaciens, in particular strain D747 (available as Double Nickel™ from Certis, US, having accession number FERM BP-8234 and disclosed in U.S. Pat. No. 7,094,592); (B1.6) Bacillus subtilis Y1336 (available as BIOBAC® WP from Bion-Tech, Taiwan, registered as a biological fungicide in Taiwan under Registration Nos. 4764, 5454, 5096 and 5277); (B1.7) Bacillus amyloliquefaciens strain MBI 600 (available as SUBTILEX from BASF SE); (B1.8) Bacillus subtilis strain GB03 (available as Kodiak® from Bayer AG, DE); (B1.9) Bacillus subtilis var. amyloliquefaciens strain FZB24 (available from Novozymes Biologicals Inc., Salem, Va. or Syngenta Crop Protection, LLC, Greensboro, N.C. as the fungicide TAEGRO® or TAEGRO® ECO (EPA Registration No. 70127-5); (B1.10) Bacillus mycoides, isolate J (available as BmJ TGAI or WG from Certis USA); (B1.11) Bacillus licheniformis, in particular strain SB3086 (available as EcoGuard™ Biofungicide and Green Releaf from Novozymes); (B1.12) a Paenibacillus sp. strain having Accession No. NRRL B-50972 or Accession No. NRRL B-67129 and described in International Patent Publication No. WO 2016/154297.

In some embodiments, the biological control agent is a Bacillus subtilis or Bacillus amyloliquefaciens strain that produces a fengycin or plipastatin-type compound, an iturin-type compound, and/or a surfactin-type compound. For background, see the following review article: Ongena, M., et al., “Bacillus Lipopeptides: Versatile Weapons for Plant Disease Biocontrol,” Trends in Microbiology, Vol 16, No. 3, March 2008, pp. 115-125. Bacillus strains capable of producing lipopeptides include Bacillus subtilis QST713 (available as SERENADE OPTI or SERENADE ASO from Bayer CropScience LP, US, having NRRL Accession No. B21661 and described in U.S. Pat. No. 6,060,051), Bacillus amyloliquefaciens strain D747 (available as Double Nickel™ from Certis, US, having accession number FERM BP-8234 and disclosed in U.S. Pat. No. 7,094,592); Bacillus subtilis MB1600 (available as SUBTILEX® from Becker Underwood, US EPA Reg. No. 71840-8); Bacillus subtilis Y1336 (available as BIOBAC® WP from Bion-Tech, Taiwan, registered as a biological fungicide in Taiwan under Registration Nos. 4764, 5454, 5096 and 5277); Bacillus amyloliquefaciens, in particular strain FZB42 (available as RHIZOVITAL® from ABiTEP, DE); and Bacillus subtilis var. amyloliquefaciens FZB24 (available from Novozymes Biologicals Inc., Salem, Va. or Syngenta Crop Protection, LLC, Greensboro, N.C. as the fungicide TAEGRO® or TAEGRO® ECO (EPA Registration No. 70127-5); and

(B2) fungi, for example: (B2.1) Coniothyrium minitans, in particular strain CON/M/91-8 (Accession No. DSM-9660; e.g. Contans® from Bayer); (B2.2) Metschnikowia fructicola, in particular strain NRRL Y-30752 (e.g. Shemer); (B2.3) Microsphaeropsis ochracea (e.g. Microx® from Prophyta); (B2.5) Trichoderma spp., including Trichoderma atroviride, strain SC1 described in International Application No. PCT/IT2008/000196); (B2.6) Trichoderma harzianum rifai strain KRL-AG2 (also known as strain T-22, /ATCC 208479, e.g. PLANTSHIELD T-22G, Rootshield®, and TurfShield from BioWorks, US); (B2.14) Gliocladium roseum, strain 321U from W.F. Stoneman Company LLC; (B2.35) Talaromyces flavus, strain V117b; (B2.36) Trichoderma asperllum, strain ICC 012 from Isagro; (B2.37) Trichoderma asperellum, strain SKT-1 (e.g. ECO-HOPE® from Kumiai Chemical Industry); (B2.38) Trichoderma atroviride, strain CNCM 1-1237 (e.g. Esquive® WP from Agrauxine, FR); (B2.39) Trichoderma atroviride, strain no. V08/002387; (B2.40) Trichoderma atroviride, strain NMI no. V08/002388; (B2.41) Trichoderma atroviride, strain NMI no. V08/002389; (B2.42) Trichoderma atroviride, strain NMI no. V08/002390; (B2.43) Trichoderma atroviride, strain LC52 (e.g. Tenet by Agrimm Technologies Limited); (B2.44) Trichoderma atroviride, strain ATCC 20476 (IMI 206040); (B2.45) Trichoderma atroviride, strain T11 (IM1352941/CECT20498); (B2.46) Trichoderma harmatum; (B2.47) Trichoderma harzianum; (B2.48) Trichoderma harzianum rifai T39 (e.g. Trichodex® from Makhteshim, US); (B2.49) Trichoderma harzianum, in particular, strain KD (e.g. Trichoplus from Biological Control Products, SA (acquired by Becker Underwood)); (B2.50) Trichoderma harzianum, strain ITEM 908 (e.g. Trianum-P from Koppert); (B2.51) Trichoderma harzianum, strain TH35 (e.g. Root-Pro by Mycontrol); (B2.52) Trichoderma virens (also known as Gliocladium virens), in particular strain GL-21 (e.g. SoilGard 12G by Certis, US); (B2.53) Trichoderma viride, strain TV1(e.g. Trianum-P by Koppert); (B2.54) Ampelomyces quisqualis, in particular strain AQ 10 (e.g. AQ 10® by IntrachemBio Italia); (B2.56) Aureobasidium pullulans, in particular blastospores of strain DSM14940; (B2.57) Aureobasidium pullulans, in particular blastospores of strain DSM 14941; (B2.58) Aureobasidium pullulans, in particular mixtures of blastospores of strains DSM14940 and DSM 14941 (e.g. Botector® by bio-ferm, CH); (B2.64) Cladosporium cladosporioides, strain H39 (by Stichting Dienst Landbouwkundig Onderzoek); (B2.69) Gliocladium catenulatum (Synonym: Clonostachys rosea f. catenulate) strain J1446 (e.g. Prestop® by AgBio Inc. and also e.g. Primastop® by Kemira Agro Oy); (B2.70) Lecanicillium lecanii (formerly known as Verticillium lecanii) conidia of strain KV01 (e.g. Vertalec® by Koppert/Arysta); (B2.71) Penicillium vermiculatum; (B2.72) Pichia anomala, strain WRL-076 (NRRL Y-30842); (B2.75) Trichoderma atroviride, strain SKT-1 (FERM P-16510); (B2.76) Trichoderma atroviride, strain SKT-2 (FERM P-16511); (B2.77) Trichoderma atroviride, strain SKT-3 (FERM P-17021); (B2.78) Trichoderma gamsii (formerly T. viride), strain ICC080 (IMI CC 392151 CABI, e.g. BioDerma by AGROBIOSOL DE MEXICO, S.A. DE C.V.); (B2.79) Trichoderma harzianum, strain DB 103 (e.g., T-Gro 7456 by Dagutat Biolab); (B2.80) Trichoderma polysporum, strain IMI 206039 (e.g. Binab TF WP by BINAB Bio-Innovation AB, Sweden); (B2.81) Trichoderma stromaticum (e.g. Tricovab by Ceplac, Brazil); (B2.83) Ulocladium oudemansii, in particular strain HRU3 (e.g. Botry-Zen® by Botry-Zen Ltd, NZ); (B2.84) Verticillium albo-atrum (formerly V. dahliae), strain WCS850 (CBS 276.92; e.g. Dutch Trig by Tree Care Innovations); (B2.86) Verticillium chlamydosporium; (B2.87) mixtures of Trichoderma asperellum strain ICC 012 and Trichoderma gamsii strain ICC 080 (product known as e.g. BIO-TAM™ from Bayer CropScience LP, US).

Further examples of biological control agents which may be combined with the compounds of formula (I) and compositions comprising thereof are:

bacteria selected from the group consisting of Bacillus cereus, in particular B. cereus strain CNCM I-1562 and Bacillus firmus, strain 1-1582 (Accession number CNCM I-1582), Bacillus subtilis strain OST 30002 (Accession No. NRRL B-50421), Bacillus thuringiensis, in particular B. thuringiensis subspecies israelensis (serotype H-14), strain AM65-52 (Accession No. ATCC 1276), B. thuringiensis subsp. aizawai, in particular strain ABTS-1857 (SD-1372), B. thuringiensis subsp. kurstaki strain HD-1, B. thuringiensis subsp. tenebrionis strain NB 176 (SD-5428), Pasteuria penetrans, Pasteuria spp. (Rotylenchulus reniformis nematode)-PR3 (Accession Number ATCC SD-5834), Streptomyces microflavus strain AQ6121 (=QRD 31.013, NRRL B-50550), and Streptomyces galbus strain AQ 6047 (Acession Number NRRL 30232);
fungi and yeasts selected from the group consisting of Beauveria bassiana, in particular strain ATCC 74040, Lecanicillium spp., in particular strain HRO LEC 12, Metarhizium anisopliae, in particular strain F52 (DSM3884 or ATCC 90448), Paecilomyces fumosoroseus (now: Isaria fumosorosea), in particular strain IFPC 200613, or strain Apopka 97 (Accession No. ATCC 20874), and Paecilomyces lilacinus, in particular P. lilacinus strain 251 (AGAL 89/030550);
viruses selected from the group consisting of Adoxophyes orana (summer fruit tortrix) granulosis virus (GV), Cydia pomonella (codling moth) granulosis virus (GV), Helicoverpa armigera (cotton bollworm) nuclear polyhedrosis virus (NPV), Spodoptera exigua (beet armyworm) mNPV, Spodoptera frugiperda (fall armyworm) mNPV, and Spodoptera littoralis (African cotton leafworm) NPV.
bacteria and fungi which can be added as ‘inoculant’ to plants or plant parts or plant organs and which, by virtue of their particular properties, promote plant growth and plant health. Examples are: Agrobacterium spp., Azorhizobium caulinodans, Azospirillum spp., Azotobacter spp., Bradyrhizobium spp., Burkholderia spp., in particular Burkholderia cepacia (formerly known as Pseudomonas cepacia), Gigaspora spp., or Gigaspora monosporum, Glomus spp., Laccaria spp., Lactobacillus buchneri, Paraglomus spp., Pisolithus tinctorus, Pseudomonas spp., Rhizobium spp., in particular Rhizobium trifolii, Rhizopogon spp., Scleroderma spp., Suillus spp., and Streptomyces spp. plant extracts and products formed by microorganisms including proteins and secondary metabolites which can be used as biological control agents, such as Allium sativum, Artemisia absinthium, azadirachtin, Biokeeper WP, Cassia nigricans, Celastrus angulatus, Chenopodium anthelminticum, chitin, Armour-Zen, Dryopteris filix-mas, Equisetum arvense, Fortune Aza, Fungastop, Heads Up (Chenopodium quinoa saponin extract), Pyrethrum/Pyrethrins, Quassia amara, Quercus, Quillaja, Regalia, “Requiem™ Insecticide”, rotenone, ryania/ryanodine, Symphytum officinale, Tanacetum vulgare, thymol, Triact 70, TriCon, Tropaeulum majus, Urtica dioica, Veratrin, Viscum album, Brassicaceae extract, in particular oilseed rape powder or mustard powder.

Examples of insecticides, acaricides and nematicides, respectively, which could be mixed with the compounds of formula (I) and compositions comprising thereof are:

(1) Acetylcholinesterase (AChE) inhibitors, such as, for example, carbamates, for example alanycarb, aldicarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, triazamate, trimethacarb, XMC and xylylcarb; or organophosphates, for example acephate, azamethiphos, azinphos-ethyl, azinphos-methyl, cadusafos, chlorethoxyfos, chlorfenvinphos, chlormephos, chlorpyrifos-methyl, coumaphos, cyanophos, demeton-S-methyl, diazinon, dichlorvos/DDVP, dicrotophos, dimethoate, dimethylvinphos, disulfoton, EPN, ethion, ethoprophos, famphur, fenamiphos, fenitrothion, fenthion, fosthiazate, heptenophos, imicyafos, isofenphos, isopropyl O-(methoxyaminothiophosphoryl) salicylate, isoxathion, malathion, mecarbam, methamidophos, methidathion, mevinphos, monocrotophos, naled, omethoate, oxydemeton-methyl, parathion-methyl, phenthoate, phorate, phosalone, phosmet, phosphamidon, phoxim, pirimiphos-methyl, profenofos, propetamphos, prothiofos, pyraclofos, pyridaphenthion, quinalphos, sulfotep, tebupirimfos, temephos, terbufos, tetrachlorvinphos, thiometon, triazophos, triclorfon and vamidothion.
(2) GABA-gated chloride channel blockers, such as, for example, cyclodiene-organochlorines, for example chlordane and endosulfan or phenylpyrazoles (fiproles), for example ethiprole and fipronil.
(3) Sodium channel modulators, such as, for example, pyrethroids, e.g. acrinathrin, allethrin, d-cis-trans allethrin, d-trans allethrin, bifenthrin, bioallethrin, bioallethrin s-cyclopentenyl isomer, bioresmethrin, cycloprothrin, cyfluthrin, beta-cyfluthrin, cyhalothrin, lambda-cyhalothrin, gamma-cyhalothrin, cypermethrin, alpha-cypermethrin, beta-cypermethrin, theta-cypermethrin, zeta-cypermethrin, cyphenothrin [(1R)-trans-isomer], deltamethrin, empenthrin [(EZ)-(1R)-isomer], esfenvalerate, etofenprox, fenpropathrin, fenvalerate, flucythrinate, flumethrin, tau-fluvalinate, halfenprox, imiprothrin, kadethrin, momfluorothrin, permethrin, phenothrin [(1R)-trans-isomer], prallethrin, pyrethrins (pyrethrum), resmethrin, silafluofen, tefluthrin, tetramethrin, tetramethrin [(1R)-isomer)], tralomethrin and transfluthrin or DDT or methoxychlor.
(4) Nicotinic acetylcholine receptor (nAChR) competitive modulators, such as, for example, neonicotinoids, e.g. acetamiprid, clothianidin, dinotefuran, imidacloprid, nitenpyram, thiacloprid and thiamethoxam or nicotine or sulfoxaflor or flupyradifurone.
(5) Nicotinic acetylcholine receptor (nAChR) allosteric modulators, such as, for example, spinosyns, e.g. spinetoram and spinosad.
(6) Glutamate-gated chloride channel (GluCl) allosteric modulators, such as, for example, avermectins/milbemycins, for example abamectin, emamectin benzoate, lepimectin and milbemectin.
(7) Juvenile hormone mimics, such as, for example, juvenile hormone analogues, e.g. hydroprene, kinoprene and methoprene or fenoxycarb or pyriproxyfen.
(8) Miscellaneous non-specific (multi-site) inhibitors, such as, for example, alkyl halides, e.g. methyl bromide and other alkyl halides; or chloropicrine or sulphuryl fluoride or borax or tartar emetic or methyl isocyanate generators, e.g. diazomet and metam.
(9) Modulators of Chordotonal Organs, such as, for example pymetrozine or flonicamid.
(10) Mite growth inhibitors, such as, for example clofentezine, hexythiazox and diflovidazin or etoxazole.
(11) Microbial disruptors of the insect gut membrane, such as, for example Bacillus thuringiensis subspecies israelensis, Bacillus sphaericus, Bacillus thuringiensis subspecies aizawai, Bacillus thuringiensis subspecies kurstaki, Bacillus thuringiensis subspecies tenebrionis, and B.t. plant proteins: Cry1Ab, Cry1Ac, Cry1Fa, Cry1A.105, Cry2Ab, Vip3A, mCry3A, Cry3Ab, Cry3Bb, Cry34Ab1/35Ab1.
(12) Inhibitors of mitochondrial ATP synthase, such as, ATP disruptors such as, for example, diafenthiuron or organotin compounds, for example azocyclotin, cyhexatin and fenbutatin oxide or propargite or tetradifon.
(13) Uncouplers of oxidative phosphorylation via disruption of the proton gradient, such as, for example, chlorfenapyr, DNOC and sulfluramid.
(14) Nicotinic acetylcholine receptor channel blockers, such as, for example, bensultap, cartap hydrochloride, thiocylam, and thiosultap-sodium.
(15) Inhibitors of chitin biosynthesis, type 0, such as, for example, bistrifluron, chlorfluazuron, diflubenzuron, flucycloxuron, flufenoxuron, hexaflumuron, lufenuron, novaluron, noviflumuron, teflubenzuron and triflumuron.
(16) Inhibitors of chitin biosynthesis, type 1, for example buprofezin.
(17) Moulting disruptor (in particular for Diptera, i.e. dipterans), such as, for example, cyromazine.
(18) Ecdysone receptor agonists, such as, for example, chromafenozide, halofenozide, methoxyfenozide and tebufenozide.
(19) Octopamine receptor agonists, such as, for example, amitraz.
(20) Mitochondrial complex III electron transport inhibitors, such as, for example, hydramethylnone or acequinocyl or fluacrypyrim.
(21) Mitochondrial complex I electron transport inhibitors, such as, for example from the group of the METI acaricides, e.g. fenazaquin, fenpyroximate, pyrimidifen, pyridaben, tebufenpyrad and tolfenpyrad or rotenone (Derris).
(22) Voltage-dependent sodium channel blockers, such as, for example indoxacarb or metaflumizone.
(23) Inhibitors of acetyl CoA carboxylase, such as, for example, tetronic and tetramic acid derivatives, e.g. spirodiclofen, spiromesifen and spirotetramat.
(24) Mitochondrial complex IV electron transport inhibitors, such as, for example, phosphines, e.g. aluminium phosphide, calcium phosphide, phosphine and zinc phosphide or cyanides, e.g. calcium cyanide, potassium cyanide and sodium cyanide.
(25) Mitochondrial complex II electron transport inhibitors, such as, for example, beta-ketonitrile derivatives, e.g. cyenopyrafen and cyflumetofen and carboxanilides, such as, for example, pyflubumide.
(28) Ryanodine receptor modulators, such as, for example, diamides, e.g. chlorantraniliprole, cyantraniliprole and flubendiamide,
further active compounds such as, for example, Afidopyropen, Afoxolaner, Azadirachtin, Benclothiaz, Benzoximate, Bifenazate, Broflanilide, Bromopropylate, Chinomethionat, Chloroprallethrin, Cryolite, Cyclaniliprole, Cycloxaprid, Cyhalodiamide, Dicloromezotiaz, Dicofol, epsilon-Metofluthrin, epsilon-Momfluthrin, Flometoquin, Fluazaindolizine, Fluensulfone, Flufenerim, Flufenoxystrobin, Flufiprole, Fluhexafon, Fluopyram, Fluralaner, Fluxametamide, Fufenozide, Guadipyr, Heptafluthrin, Imidaclothiz, Iprodione, kappa-Bifenthrin, kappa-Tefluthrin, Lotilaner, Meperfluthrin, Paichongding, Pyridalyl, Pyrifluquinazon, Pyriminostrobin, Spirobudiclofen, Tetramethylfluthrin, Tetraniliprole, Tetrachlorantraniliprole, Tigolaner, Tioxazafen, Thiofluoximate, Triflumezopyrim and iodomethane; furthermore preparations based on Bacillus firmus (1-1582, BioNeem, Votivo), and also the following compounds: 1-{2-fluoro-4-methyl-5-[(2,2,2-trifluoroethyl)sulphinyl]phenyl}-3-(trifluoromethyl)-1H-1,2,4-triazole-5-amine (known from WO2006/043635) (CAS 885026-50-6), {1′-[(2E)-3-(4-chlorophenyl)prop-2-en-1-yl]-5-fluorospiro[indol-3,4′-piperidin]-1(2H)-yl}(2-chloropyridin-4-yl)methanone (known from WO2003/106457) (CAS 637360-23-7), 2-chloro-N-[2-{i-[(2E)-3-(4-chlorophenyl)prop-2-en-1-yl]piperidin-4-yl}-4-(trifluoromethyl)phenyl]isonicotinamide (known from WO2006/003494) (CAS 872999-66-1), 3-(4-chloro-2,6-dimethylphenyl)-4-hydroxy-8-methoxy-1,8-diazaspiro[4.5]dec-3-en-2-one (known from WO 2010052161) (CAS 1225292-17-0), 3-(4-chloro-2,6-dimethylphenyl)-8-methoxy-2-oxo-1,8-diazaspiro[4.5]dec-3-en-4-yl ethyl carbonate (known from EP2647626) (CAS 1440516-42-6), 4-(but-2-yn-1-yloxy)-6-(3,5-dimethylpiperidin-1-yl)-5-fluoropyrimidine (known from WO2004/099160) (CAS 792914-58-0), PF1364 (known from JP2010/018586) (CAS 1204776-60-2), N-[(2E)-1-[(6-chloropyridin-3-yl)methyl]pyridin-2(1H)-ylidene]-2,2,2-trifluoroacetamide (known from WO2012/029672) (CAS 1363400-41-2), (3E)-3-[1-[(6-chloro-3-pyridyl)methyl]-2-pyridylidene]-1,1,1-trifluoro-propan-2-one (known from WO2013/144213) (CAS 1461743-15-6), N-[3-(benzylcarbamoyl)-4-chlorophenyl]-1-methyl-3-(pentafluoroethyl)-4-(trifluoromethyl)-1H-pyrazole-5-carboxamide (known from WO2010/051926) (CAS 1226889-14-0), 5-bromo-4-chloro-N-[4-chloro-2-methyl-6-(methylcarbamoyl)phenyl]-2-(3-chloro-2-pyridyl)pyrazole-3-carboxamide (known from CN103232431) (CAS 1449220-44-3), 4-[5-(3,5-dichlorophenyl)-4,5-dihydro-5-(trifluoromethyl)-3-isoxazolyl]-2-methyl-N-(cis-1-oxido-3-thietanyl)-benzamide, 4-[5-(3,5-dichlorophenyl)-4,5-dihydro-5-(trifluoromethyl)-3-isoxazolyl]-2-methyl-N-(trans-1-oxido-3-thietanyl)-benzamide and 4-[(5S)-5-(3,5-dichlorophenyl)-4,5-dihydro-5-(trifluoromethyl)-3-isoxazolyl]-2-methyl-N-(cis-1-oxido-3-thietanyl)benzamide (known from WO 2013/050317 A) (CAS 1332628-83-7), N-[3-chloro-1-(3-pyridinyl)-1H-pyrazol-4-yl]-N-ethyl-3-[(3, 3,3-trifluoropropyl)sulfinyl]-propanamide, (+)-N-[3-chloro-1-(3-pyridinyl)-1H-pyrazol-4-yl]-N-ethyl-3-[(3, 3,3-trifluoropropyl)sulfinyl]-propanamide and (−)-N-[3-chloro-1-(3-pyridinyl)-H-pyrazol-4-yl]-N-ethyl-3-[(3,3,3-trifluoropropyl)sulfinyl]-propanamide (known from WO2013/162715A2, WO2013/162716A2, US 2014/0213448 A1) (CAS 1477923-37-7), 5-[[(2E)-3-chloro-2-propen-1-yl]amino]-1-[2,6-dichloro-4-(trifluoromethyl)phenyl]-4-[(trifluoromethyl)sulfinyl]-1H-pyrazole-3-carbonitrile (known from CN 101337937A) (CAS 1105672-77-2), 3-bromo-N-[4-chloro-2-methyl-6-[(methylamino)thioxomethyl]phenyl]-1-(3-chloro-2-pyridinyl)-1H-pyrazole-5-carboxamide, (Liudaibenjiaxuanan, known from CN 103109816A) (CAS 1232543-85-9); N-[4-chloro-2-[[(1,1-dimethylethyl)amino]carbonyl]-6-methylphenyl]-1-(3-chloro-2-pyridinyl)-3-(fluoromethoxy)-1H-Pyrazole-5-carboxamide (known from WO 2012/034403 A1) (CAS 1268277-22-0), N-[2-(5-amino-1,3,4-thiadiazol-2-yl)-4-chloro-6-methylphenyl]-3-bromo-1-(3-chloro-2-pyridinyl)-1H-pyrazole-5-carboxamide (known from WO 2011/085575 A1) (CAS 1233882-22-8), 4-[3-[2,6-dichloro-4-[(3,3-dichloro-2-propen-1-yl)oxy]phenoxy]propoxy]-2-methoxy-6-(trifluoromethyl)-pyrimidine (known from CN 101337940 A) (CAS 1108184-52-6); (2E)- and 2(Z)-2-[2-(4-cyanophenyl)-1-[3-(trifluoromethyl)phenyl]ethylidene]-N-[4-(difluoromethoxy)phenyl]-hydrazinecarboxamide (known from CN 101715774 A) (CAS 1232543-85-9); 3-(2,2-dichloroethenyl)-2,2-dimethyl-4-(1H-benzimidazol-2-yl)phenyl-cyclopropanecarboxylic acid ester (known from CN 103524422 A) (CAS 1542271-46-4); (4aS)-7-chloro-2,5-dihydro-2-[[(methoxycarbonyl)[4-[(trifluoromethyl)thio]phenyl]amino]carbonyl]-indeno[1,2-e][1,3,4]oxadiazine-4a(3H)-carboxylic acid methyl ester (known from CN 102391261 A) (CAS 1370358-69-2); 6-deoxy-3-O-ethyl-2,4-di-O-methyl-, 1-[N-[4-[1-[4-(1,1,2,2,2-pentafluoroethoxy)phenyl]-1H-1,2,4-triazol-3-yl]phenyl]carbamate]-α-L-mannopyranose (known from US 2014/0275503 A1) (CAS 1181213-14-8); 8-(2-cyclopropylmethoxy-4-trifluoromethyl-phenoxy)-3-(6-trifluoromethyl-pyridazin-3-yl)-3-aza-bicyclo[3.2.1]octane (CAS 1253850-56-4), (8-anti)-8-(2-cyclopropylmethoxy-4-trifluoromethyl-phenoxy)-3-(6-trifluoromethyl-pyridazin-3-yl)-3-aza-bicyclo[3.2.1]octane (CAS 933798-27-7), (8-syn)-8-(2-cyclopropylmethoxy-4-trifluoromethyl-phenoxy)-3-(6-trifluoromethyl-pyridazin-3-yl)-3-aza-bicyclo[3.2.1]octane (known from WO 2007040280 A1, WO 2007040282 A1) (CAS 934001-66-8), N-[3-chloro-1-(3-pyridinyl)-1H-pyrazol-4-yl]-N-ethyl-3-[(3,3,3-trifluoropropyl)thio]-propanamide (known from WO 2015/058021 A1, WO 2015/058028 A1) (CAS 1477919-27-9) and N-[4-(aminothioxomethyl)-2-methyl-6-[(methylamino)carbonyl]phenyl]-3-bromo-1-(3-chloro-2-pyridinyl)-1H-pyrazole-5-carboxamide (known from CN 103265527 A) (CAS 1452877-50-7), 5-(1,3-dioxan-2-yl)-4-[[4-(trifluoromethyl)phenyl]methoxy]-pyrimidine (known from WO 2013/115391 A1) (CAS 1449021-97-9), 3-(4-chloro-2,6-dimethylphenyl)-4-hydroxy-8-methoxy-1-methyl-1,8-diazaspiro[4.5]dec-3-en-2-one (known from WO 2010/066780 A1, WO 2011/151146 A1) (CAS 1229023-34-0), 3-(4-chloro-2,6-dimethylphenyl)-8-methoxy-1-methyl-1,8-diazaspiro[4.5]decane-2,4-dione (known from WO 2014/187846 A1) (CAS 1638765-58-8), 3-(4-chloro-2,6-dimethylphenyl)-8-methoxy-1-methyl-2-oxo-1, 8-diazaspiro[4.5]dec-3-en-4-yl-carbonic acid ethyl ester (known from WO 2010/066780 A1, WO 2011151146 A1) (CAS 1229023-00-0), N-[1-[(6-chloro-3-pyridinyl)methyl]-2(1H)-pyridinylidene]-2,2,2-trifluoro-acetamide (known from DE 3639877 A1, WO 2012029672 A1) (CAS 1363400-41-2), [N(E)]-N-[1-[(6-chloro-3-pyridinyl)methyl]-2(1H)-pyridinylidene]-2,2,2-trifluoro-acetamide, (known from WO 2016005276 A1) (CAS 1689566-03-7), [N(Z)]—N-[1-[(6-chloro-3-pyridinyl)methyl]-2(1H)-pyridinylidene]-2,2,2-trifluoro-acetamide, (CAS 1702305-40-5), 3-endo-3-[2-propoxy-4-(trifluoromethyl)phenoxy]-9-[[5-(trifluoromethyl)-2-pyridinyl]oxy]-9-azabicyclo[3.3.1]nonane (known from WO 2011/105506 A1, WO 2016/133011 A1) (CAS 1332838-17-1).

Examples of safeners which could be mixed with the compounds of formula (I) and compositions comprising thereof are, for example, benoxacor, cloquintocet (-mexyl), cyometrinil, cyprosulfamide, dichlormid, fenchlorazole (-ethyl), fenclorim, flurazole, fluxofenim, furilazole, isoxadifen (-ethyl), mefenpyr (-diethyl), naphthalic anhydride, oxabetrinil, 2-methoxy-N-({4-[(methylcarbamoyl)amino]phenyl}-sulphonyl)benzamide (CAS 129531-12-0), 4-(dichloroacetyl)-1-oxa-4-azaspiro[4.5]decane (CAS 71526-07-3),2,2,5-trimethyl-3-(dichloroacetyl)-1,3-oxazolidine(CAS52836-31-4).

Examples of herbicides which could be mixed with the compounds of formula (I) and compositions comprising thereof are:

Acetochlor, acifluorfen, acifluorfen-sodium, aclonifen, alachlor, allidochlor, alloxydim, alloxydim-sodium, ametryn, amicarbazone, amidochlor, amidosulfuron, 4-amino-3-chloro-6-(4-chloro-2-fluoro-3-methylphenyl)-5-fluoropyridine-2-carboxylic acid, aminocyclopyrachlor, aminocyclopyrachlor-potassium, aminocyclopyrachlor-methyl, aminopyralid, amitrole, ammoniumsulfamate, anilofos, asulam, atrazine, azafenidin, azimsulfuron, beflubutamid, benazolin, benazolin-ethyl, benfluralin, benfuresate, bensulfuron, bensulfuron-methyl, bensulide, bentazone, benzobicyclon, benzofenap, bicyclopyron, bifenox, bilanafos, bilanafos-sodium, bispyribac, bispyribac-sodium, bromacil, bromobutide, bromofenoxim, bromoxynil, bromoxynil-butyrate, -potassium, -heptanoate, and -octanoate, busoxinone, butachlor, butafenacil, butamifos, butenachlor, butralin, butroxydim, butylate, cafenstrole, carbetamide, carfentrazone, carfentrazone-ethyl, chloramben, chlorbromuron, chlorfenac, chlorfenac-sodium, chlorfenprop, chlorflurenol, chlorflurenol-methyl, chloridazon, chlorimuron, chlorimuron-ethyl, chlorophthalim, chlorotoluron, chlorthal-dimethyl, chlorsulfuron, cinidon, cinidon-ethyl, cinmethylin, cinosulfuron, clacyfos, clethodim, clodinafop, clodinafop-propargyl, clomazone, clomeprop, clopyralid, cloransulam, cloransulam-methyl, cumyluron, cyanamide, cyanazine, cycloate, cyclopyrimorate, cyclosulfamuron, cycloxydim, cyhalofop, cyhalofop-butyl, cyprazine, 2,4-D, 2,4-D-butotyl, -butyl, -dimethylammonium, -diolamin, -ethyl, -2-ethylhexyl, -isobutyl, -isooctyl, -isopropyl-ammonium, -potassium, -triisopropanolammonium, and -trolamine, 2,4-DB, 2,4-DB-butyl, -dimethyl-ammonium, -isooctyl, -potassium, and -sodium, daimuron (dymron), dalapon, dazomet, n-decanol, desmedipham, detosyl-pyrazolate (DTP), dicamba, dichlobenil, 2-(2,4-dichlorobenzyl)-4,4-dimethyl-1,2-oxazolidin-3-one, 2-(2,5-dichlorobenzyl)-4,4-dimethyl-1,2-oxazolidin-3-one, dichlorprop, dichlorprop-P, diclofop, diclofop-methyl, diclofop-P-methyl, diclosulam, difenzoquat, diflufenican, diflufenzopyr, diflufenzopyr-sodium, dimefuron, dimepiperate, dimethachlor, dimethametryn, dimethenamid, dimethenamid-P, dimetrasulfuron, dinitramine, dinoterb, diphenamid, diquat, diquat-dibromid, dithiopyr, diuron, DNOC, endothal, EPTC, esprocarb, ethalfluralin, ethametsulfuron, etha-metsulfuron-methyl, ethiozin, ethofumesate, ethoxyfen, ethoxyfen-ethyl, ethoxysulfuron, etobenzanid, F-9600, F-5231, i.e. N-{2-chloro-4-fluoro-5-[4-(3-fluoropropyl)-5-oxo-4,5-dihydro-1H-tetrazol-1-yl]phenyl}ethanesulfonamide, F-7967, i. e. 3-[7-chloro-5-fluoro-2-(trifluoromethyl)-1H-benzimidazol-4-yl]-1-methyl-6-(trifluoromethyl)pyrimidine-2,4(1H,3H)-dione, fenoxaprop, fenoxaprop-P, fenoxaprop-ethyl, fenoxaprop-P-ethyl, fenoxasulfone, fenquinotrione, fentrazamide, flamprop, flamprop-M-isopropyl, flamprop-M-methyl, flazasulfuron, florasulam, fluazifop, fluazifop-P, fluazifop-butyl, fluazifop-P-butyl, flucarbazone, flucarbazone-sodium, flucetosulfuron, fluchloralin, flufenacet, flufenpyr, flufenpyr-ethyl, flumetsulam, flumiclorac, flumiclorac-pentyl, flumioxazin, fluometuron, flurenol, flurenol-butyl, -dimethylammonium and -methyl, fluoroglycofen, fluoroglycofen-ethyl, flupropanate, flupyr-sulfuron, flupyrsulfuron-methyl-sodium, fluridone, flurochloridone, fluroxypyr, fluroxypyr-meptyl, flurtamone, fluthiacet, fluthiacet-methyl, fomesafen, fomesafen-sodium, foramsulfuron, fosamine, glufosinate, glufosinate-ammonium, glufosinate-P-sodium, glufosinate-P-ammonium, glufosinate-P-sodium, glyphosate, glyphosate-ammonium, -isopropylammonium, -diammonium, -dimethylammonium, -potassium, -sodium, and -trimesium, H-9201, i.e. O-(2,4-dimethyl-6-nitrophenyl) O-ethyl isopropylphosphoramidothioate, halauxifen, halauxifen-methyl halosafen, halosulfuron, halosulfuron-methyl, haloxyfop, haloxyfop-P, haloxyfop-ethoxyethyl, haloxyfop-P-ethoxyethyl, haloxyfop-methyl, haloxyfop-P-methyl, hexazinone, HW-02, i.e. 1-(dimethoxyphosphoryl) ethyl-(2,4-dichlorophenoxy)acetate, imazamethabenz, imazamethabenz-methyl, imazamox, imazamox-ammonium, imazapic, imazapic-ammonium, imazapyr, imazapyr-isopropylammonium, imazaquin, imazaquin-ammonium, imazethapyr, imazethapyr-immonium, imazosulfuron, indanofan, indaziflam, iodosulfuron, iodosulfuron-methyl-sodium, ioxynil, ioxynil-octanoate, -potassium and -sodium, ipfencarbazone, isoproturon, isouron, isoxaben, isoxaflutole, karbutilate, KUH-043, i.e. 3-({[5-(difluoromethyl)-1-methyl-3-(trifluoromethyl)-1H-pyrazol-4-yl]methyl}sulfonyl)-5,5-dimethyl-4,5-dihydro-1,2-oxazole, ketospiradox, lactofen, lenacil, linuron, MCPA, MCPA-butotyl, -dimethylammonium, -2-ethylhexyl, -isopropylammonium, -potassium, and -sodium, MCPB, MCPB-methyl, -ethy,l and -sodium, mecoprop, mecoprop-sodium, and -butotyl, mecoprop-P, mecoprop-P-butotyl, -dimethylammonium, -2-ethylhexyl, and -potassium, mefenacet, mefluidide, mesosulfuron, mesosulfuron-methyl, mesotrione, methabenzthiazuron, metam, metamifop, metamitron, metazachlor, metazosulfuron, methabenz-thiazuron, methiopyrsulfuron, methiozolin, methyl isothiocyanate, metobromuron, metolachlor, S-metolachlor, metosulam, metoxuron, metribuzin, metsulfuron, metsulfuron-methyl, molinat, mono-linuron, monosulfuron, monosulfuron-ester, MT-5950, i.e. N-(3-chloro-4-isopropylphenyl)-2-methylpentan amide, NGGC-011, napropamide, NC-310, i.e. [5-(benzyloxy)-1-methyl-1H-pyrazol-4-yl](2,4-dichlorophenyl)methanone, neburon, nicosulfuron, nonanoic acid (pelargonic acid), norflurazon, oleic acid (fatty acids), orbencarb, orthosulfamuron, oryzalin, oxadiargyl, oxadiazon, oxasulfuron, oxaziclomefon, oxyfluorfen, paraquat, paraquat dichloride, pebulate, pendimethalin, penoxsulam, pentachlorphenol, pentoxazone, pethoxamid, petroleum oils, phenmedipham, picloram, picolinafen, pinoxaden, piperophos, pretilachlor, primisulfuron, primisulfuron-methyl, prodiamine, profoxydim, prometon, prometryn, propachlor, propanil, propaquizafop, propazine, propham, propisochlor, propoxycarbazone, propoxycarbazone-sodium, propyrisulfuron, propyzamide, prosulfocarb, pro-sulfuron, pyraclonil, pyraflufen, pyraflufen-ethyl, pyrasulfotole, pyrazolynate (pyrazolate), pyrazo-sulfuron, pyrazosulfuron-ethyl, pyrazoxyfen, pyribambenz, pyribambenz-isopropyl, pyribambenz-propyl, pyribenzoxim, pyributicarb, pyridafol, pyridate, pyriftalid, pyriminobac, pyriminobac-methyl, pyrimisulfan, pyrithiobac, pyrithiobac-sodium, pyroxasulfone, pyroxsulam, quinclorac, quinmerac, quinoclamine, quizalofop, quizalofop-ethyl, quizalofop-P, quizalofop-P-ethyl, quizalofop-P-tefuryl, rimsulfuron, saflufenacil, sethoxydim, siduron, simazine, simetryn, SL-261, sulcotrion, sulfentrazone, sulfometuron, sulfometuron-methyl, sulfosulfuron, SYN-523, SYP-249, i.e. 1-ethoxy-3-methyl-1-oxobut-3-en-2-yl 5-[2-chloro-4-(trifluoromethyl)phenoxy]-2-nitrobenzoate, SYP-300, i.e. 1-[7-fluoro-3-oxo-4-(prop-2-yn-1-yl)-3,4-dihydro-2H-1,4-benzoxazin-6-yl]-3-propyl-2-thioxoimidazolidine-4,5-dione, 2,3,6-TBA, TCA (trichloroacetic acid), TCA-sodium, tebuthiuron, tefuryltrione, tembotrione, tepraloxydim, terbacil, terbucarb, terbumeton, terbuthylazin, terbutryn, thenylchlor, thiazopyr, thien-carbazone, thiencarbazone-methyl, thifensulfuron, thifensulfuron-methyl, thiobencarb, tiafenacil, tolpyralate, topramezone, tralkoxydim, triafamone, tri-allate, triasulfuron, triaziflam, tribenuron, tribenuron-methyl, triclopyr, trietazine, trifloxysulfuron, trifloxysulfuron-sodium, trifludimoxazin, trifluralin, triflusulfuron, triflusulfuron-methyl, tritosulfuron, urea sulfate, vernolate, XDE-848, ZJ-0862, i.e. 3,4-dichloro-N-{2-[(4,6-dimethoxypyrimidin-2-yl)oxy]benzyl}aniline, and the following compounds:

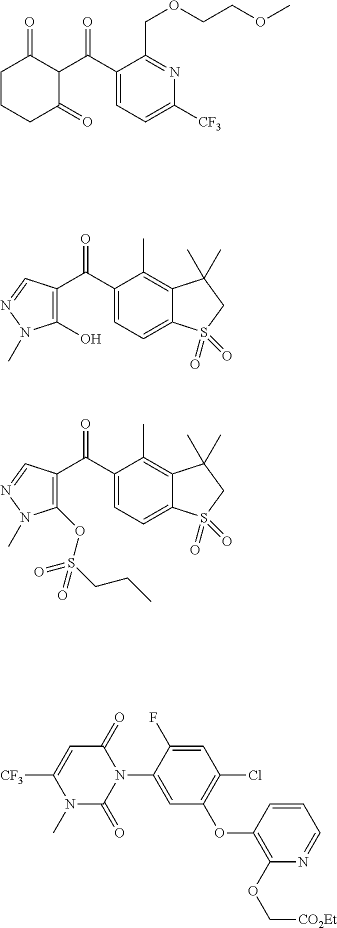 [SMILES: COCCOCc1nc(C(F)(F)F)ccc1C(=O)C1C(=O)CCCC1=O ; Cc1c(C(=O)c2cnn(C)c2O)ccc2c1C(C)(C)CS2(=O)=O ; CCCS(=O)(=O)Oc1c(C(=O)c2ccc3c(c2C)C(C)(C)CS3(=O)=O)cnn1C ; CCOC(=O)COc1ncccc1Oc1cc(-n2c(=O)cc(C(F)(F)F)n(C)c2=O)c(F)cc1Cl]

### Examples for Plant Growth Regulators are:

Acibenzolar, acibenzolar-S-methyl, 5-aminolevulinic acid, ancymidol, 6-benzylaminopurine, Brassinolid, catechine, chlormequat chloride, cloprop, cyclanilide, 3-(cycloprop-1-enyl) propionic acid, daminozide, dazomet, n-decanol, dikegulac, dikegulac-sodium, endothal, endothal-dipotassium, -disodium, and -mono(N,N-dimethylalkylammonium), ethephon, flumetralin, flurenol, flurenol-butyl, flurprimidol, forchlorfenuron, gibberellic acid, inabenfide, indol-3-acetic acid (IAA), 4-indol-3-ylbutyric acid, isoprothiolane, probenazole, jasmonic acid, maleic hydrazide, mepiquat chloride, 1-methyl-cyclopropene, methyl jasmonate, 2-(1-naphthyl)acetamide, 1-naphthylacetic acid, 2-naphthyloxyacetic acid, nitrophenolate-mixture, paclobutrazol, N-(2-phenylethyl)-beta-alanine, N-phenylphthalamic acid, prohexadione, prohexadione-calcium, prohydrojasmone, salicylic acid, strigolactone, tecnazene, thidiazuron, triacontanol, trinexapac, trinexapac-ethyl, tsitodef, uniconazole, uniconazole-P.

### Methods and Uses

The compounds of formula (I) and the compositions comprising thereof have potent microbicidal activity. They can be used for controlling unwanted microorganisms, such as unwanted fungi and bacteria. They can be particularly useful in crop protection (they control microorganisms that cause plants diseases) or for protecting materials (e.g. industrial materials, timber, storage goods) as described in more details herein below. More specifically, the compounds of formula (I) and the composition of the invention can be used to protect seeds, germinating seeds, emerged seedlings, plants, plant parts, fruits, harvest goods and/or the soil in which the plants grow from unwanted microorganisms.

Control or controlling as used herein encompasses protective, curative and eradicative treatment of unwanted microorganisms. Unwanted microorganisms may be pathogenic bacteria, pathogenic virus, pathogenic oomycetes or pathogenic fungi, more specifically phytopathogenic bacteria phytopathogenic virus, phytopathogenic oomycetes or phytopathogenic fungi. As detailed herein below, these phytopathogenic microorganims are the causal agents of a broad spectrum of plants diseases.

More specifically, the compound of formula (I) and the composition of the invention can be used as fungicides. For the purpose of the specification, the term “fungicide” refers to a compound or composition that can be used in crop protection for the control of unwanted fungi, such as Plasmodiophoromycetes, Chytridiomycetes, Zygomycetes, Ascomycetes, Basidiomycetes and Deuteromycetes and/or for the control of Oomycetes, more preferably for the control of Basidiomycetes (causing rust diseases).

The present invention also relates to a method for controlling unwanted microorganisms, such as phytopathogenic fungi, oomycetes and bacteria, comprising the step of applying at least one compound of formula (I) or at least one composition of the invention to the microorganisms and/or their habitat (to the plants, plant parts, seeds, fruits or to the soil in which the plants grow).

Typically, when the compound and the composition of the invention are used in curative or protective methods for controlling phytopathogenic fungi and/or phytopathogenic oomycetes, an effective and plant-compatible amount thereof is applied to the plants, plant parts, fruits, seeds or to the soil or substrates in which the plants grow. Suitable substrates that may be used for cultivating plants include inorganic based substrates, such as mineral wool, in particular stone wool, perlite, sand or gravel; organic substrates, such as peat, pine bark or sawdust; and petroleum based substrates such as polymeric foams or plastic beads. Effective and plant-compatible amount means an amount that is sufficient to control or destroy the fungi present or liable to appear on the cropland and that does not entail any appreciable symptom of phytotoxicity for said crops. Such an amount can vary within a wide range depending on the fungus to be controlled, the type of crop, the crop growth stage, the climatic conditions and the respective compound or composition of the invention used. This amount can be determined by systematic field trials that are within the capabilities of a person skilled in the art.

### Plants and Plant Parts

The compounds of formula (I) and compositions comprising thereof may be applied to any plants or plant parts.

Plants mean all plants and plant populations, such as desired and undesired wild plants or crop plants (including naturally occurring crop plants). Crop plants may be plants which can be obtained by conventional breeding and optimization methods or by biotechnological and genetic engineering methods or combinations of these methods, including the genetically modified plants (GMO or transgenic plants) and the plant cultivars which are protectable and non-protectable by plant breeders' rights.

Plant parts are understood to mean all parts and organs of plants above and below the ground, such as shoot, leaf, flower and root, examples of which include leaves, needles, stalks, stems, flowers, fruit bodies, fruits and seeds, and also roots, tubers and rhizomes. The plant parts also include harvested material and vegetative and generative propagation material, for example cuttings, tubers, rhizomes, slips and seeds.

Plants which may be treated in accordance with the methods of the invention include the following: cotton, flax, grapevine, fruit, vegetables, such as Rosaceae sp. (for example pome fruits such as apples and pears, but also stone fruits such as apricots, cherries, almonds and peaches, and soft fruits such as strawberries), Ribesioidae sp., Juglandaceae sp., Betulaceae sp., Anacardiaceae sp., Fagaceae sp., Moraceae sp., Oleaceae sp., Actinidaceae sp., Lauraceae sp., Musaceae sp. (for example banana trees and plantations), Rubiaceae sp. (for example coffee), Theaceae sp., Sterculiceae sp., Rutaceae sp. (for example lemons, oranges and grapefruit); Solanaceae sp. (for example tomatoes), Liliaceae sp., Asteraceae sp. (for example lettuce), Umbelliferae sp., Cruciferae sp., Chenopodiaceae sp., Cucurbitaceae sp. (for example cucumber), Alliaceae sp. (for example leek, onion), Papilionaceae sp. (for example peas); major crop plants, such as Gramineae sp. (for example maize, turf, cereals such as wheat, rye, rice, barley, oats, millet and triticale), Asteraceae sp. (for example sunflower), Brassicaceae sp. (for example white cabbage, red cabbage, broccoli, cauliflower, Brussels sprouts, pak choi, kohlrabi, radishes, and oilseed rape, mustard, horseradish and cress), Fabacae sp. (for example bean, peanuts), Papilionaceae sp. (for example soya bean), Solanaceae sp. (for example potatoes), Chenopodiaceae sp. (for example sugar beet, fodder beet, swiss chard, beetroot); useful plants and ornamental plants for gardens and wooded areas; and genetically modified varieties of each of these plants.

In some preferred embodiments, wild plant species and plant cultivars, or those obtained by conventional biological breeding methods, such as crossing or protoplast fusion, and also parts thereof, are treated in accordance with the methods of the invention.

In some other preferred embodiments, transgenic plants and plant cultivars obtained by genetic engineering methods, if appropriate in combination with conventional methods (Genetically Modified Organisms), and parts thereof are treated in accordance with the methods of the invention. More preferably, plants of the plant cultivars which are commercially available or are in use are treated in accordance with the invention. Plant cultivars are understood to mean plants which have new properties (“traits”) and have been obtained by conventional breeding, by mutagenesis or by recombinant DNA techniques. They can be cultivars, varieties, bio- or genotypes.

The methods according to the invention can be used in the treatment of genetically modified organisms (GMOs), e.g. plants or seeds. Genetically modified plants (or transgenic plants) are plants of which a heterologous gene has been stably integrated into genome. The expression “heterologous gene” essentially means a gene which is provided or assembled outside the plant and when introduced in the nuclear, chloroplastic or mitochondrial genome gives the transformed plant new or improved agronomic or other properties by expressing a protein or polypeptide of interest or by downregulating or silencing other gene(s) which are present in the plant (using for example, antisense technology, cosuppression technology, RNA interference—RNAi—technology or microRNA—miRNA—technology). A heterologous gene that is located in the genome is also called a transgene. A transgene that is defined by its particular location in the plant genome is called a transformation or transgenic event.

Plants and plant cultivars which can be treated by the above disclosed methods include all plants which have genetic material which impart particularly advantageous, useful traits to these plants (whether obtained by breeding and/or biotechnological means).

Plants and plant cultivars which can be treated by the above disclosed methods include plants and plant cultivars which are resistant against one or more biotic stresses, i.e. said plants show a better defense against animal and microbial pests, such as against nematodes, insects, mites, phytopathogenic fungi, bacteria, viruses and/or viroids.

Plants and plant cultivars which can be treated by the above disclosed methods include those plants which are resistant to one or more abiotic stresses. Abiotic stress conditions may include, for example, drought, cold temperature exposure, heat exposure, osmotic stress, flooding, increased soil salinity, increased mineral exposure, ozone exposure, high light exposure, limited availability of nitrogen nutrients, limited availability of phosphorus nutrients, shade avoidance.

Plants and plant cultivars which can be treated by the above disclosed methods include those plants characterized by enhanced yield characteristics. Increased yield in said plants can be the result of, for example, improved plant physiology, growth and development, such as water use efficiency, water retention efficiency, improved nitrogen use, enhanced carbon assimilation, improved photosynthesis, increased germination efficiency and accelerated maturation. Yield can furthermore be affected by improved plant architecture (under stress and non-stress conditions), including but not limited to, early flowering, flowering control for hybrid seed production, seedling vigor, plant size, internode number and distance, root growth, seed size, fruit size, pod size, pod or ear number, seed number per pod or ear, seed mass, enhanced seed filling, reduced seed dispersal, reduced pod dehiscence and lodging resistance.

Further yield traits include seed composition, such as carbohydrate content and composition for example cotton or starch, protein content, oil content and composition, nutritional value, reduction in anti-nutritional compounds, improved processability and better storage stability.

Plants and plant cultivars which can be treated by the above disclosed methods include plants and plant cultivars which are hybrid plants that already express the characteristic of heterosis or hybrid vigor which results in generally higher yield, vigor, health and resistance towards biotic and abiotic stresses.

Plants and plant cultivars (obtained by plant biotechnology methods such as genetic engineering) which can be treated by the above disclosed methods include plants and plant cultivars which are herbicide-tolerant plants, i.e. plants made tolerant to one or more given herbicides. Such plants can be obtained either by genetic transformation, or by selection of plants containing a mutation imparting such herbicide tolerance.

Plants and plant cultivars (obtained by plant biotechnology methods such as genetic engineering) which can be treated by the above disclosed methods include plants and plant cultivars which are insect-resistant transgenic plants, i.e. plants made resistant to attack by certain target insects. Such plants can be obtained by genetic transformation, or by selection of plants containing a mutation imparting such insect resistance.

Plants and plant cultivars (obtained by plant biotechnology methods such as genetic engineering) which can be treated by the above disclosed methods include plants and plant cultivars which are tolerant to abiotic stresses. Such plants can be obtained by genetic transformation, or by selection of plants containing a mutation imparting such stress resistance.

Plants and plant cultivars (obtained by plant biotechnology methods such as genetic engineering) which can be treated by the above disclosed methods include plants and plant cultivars which show altered quantity, quality and/or storage-stability of the harvested product and/or altered properties of specific ingredients of the harvested product.

Plants and plant cultivars (obtained by plant biotechnology methods such as genetic engineering) which can be treated by the above disclosed methods include plants and plant cultivars, such as cotton plants, with altered fiber characteristics. Such plants can be obtained by genetic transformation, or by selection of plants contain a mutation imparting such altered fiber characteristics.

Plants and plant cultivars (obtained by plant biotechnology methods such as genetic engineering) which can be treated by the above disclosed methods include plants and plant cultivars, such as oilseed rape or related Brassica plants, with altered oil profile characteristics. Such plants can be obtained by genetic transformation, or by selection of plants contain a mutation imparting such altered oil profile characteristics.

Plants and plant cultivars (obtained by plant biotechnology methods such as genetic engineering) which can be treated by the above disclosed methods include plants and plant cultivars, such as oilseed rape or related Brassica plants, with altered seed shattering characteristics. Such plants can be obtained by genetic transformation, or by selection of plants contain a mutation imparting such altered seed shattering characteristics and include plants such as oilseed rape plants with delayed or reduced seed shattering.

Plants and plant cultivars (obtained by plant biotechnology methods such as genetic engineering) which can be treated by the above disclosed methods include plants and plant cultivars, such as Tobacco plants, with altered post-translational protein modification patterns.

### Pathogens and Diseases

The methods disclosed above can be used to control microorganisms, in particular phytopathogenic microorganisms such as phytopathogenic fungi, causing diseases, such as:

diseases caused by powdery mildew pathogens, such as Blumeria species (e.g. Blumeria graminis), Podosphaera species (e.g. Podosphaera leucotricha), Sphaerotheca species (e.g. Sphaerotheca fuliginea), Uncinula species (e.g. Uncinula necator);
diseases caused by rust disease pathogens, such as Gymnosporangium species (e.g. Gymnosporangium sabinae), Hemileia species (e.g. Hemileia vastatrix), Phakopsora species (e.g. Phakopsora pachyrhizi or Phakopsora meibomiae), Puccinia species (e.g. Puccinia recondita, Puccinia graminis or Puccinia striiformis), Uromyces species (e.g. Uromyces appendiculatus);
diseases caused by pathogens from the group of the Oomycetes, such as Albugo species (e.g. Albugo candida), Bremia species (e.g. Bremia lactucae), Peronospora species (e.g. Peronospora pisi or P. brassicae), Phytophthora species (e.g. Phytophthora infestans), Plasmopara species (e.g. Plasmopara viticola), Pseudoperonospora species (e.g. Pseudoperonospora humuli or Pseudoperonospora cubensis), Pythium species (e.g. Pythium ultimum);
leaf blotch diseases and leaf wilt diseases caused, for example, by Alternaria species (e.g. Alternaria solani), Cercospora species (e.g. Cercospora beticola), Cladiosporium species (e.g. Cladiosporium cucumerinum), Cochliobolus species (e.g. Cochliobolus sativus (conidial form: Drechslera, syn: Helminthosporium) or Cochliobolus miyabeanus), Colletotrichum species (e.g. Colletotrichum lindemuthanium), Cycloconium species (e.g. Cycloconium oleaginum), Diaporthe species (e.g. Diaporthe citri), Elsinoe species (e.g. Elsinoe fawcettii), Gloeosporium species (e.g. Gloeosporium laeticolor), Glomerella species (e.g. Glomerella cingulate), Guignardia species (e.g. Guignardia bidwelli), Leptosphaeria species (e.g. Leptosphaeria maculans), Magnaporthe species (e.g. Magnaporthe grisea), Microdochium species (e.g. Microdochium nivale), Mycosphaerella species (e.g. Mycosphaerella graminicola, Mycosphaerella arachidicola or Mycosphaerella fijiensis), Phaeosphaeria species (e.g. Phaeosphaeria nodorum), Pyrenophora species (e.g. Pyrenophora teres or Pyrenophora tritici repentis), Ramularia species (e.g. Ramularia collo-cygni or Ramularia areola), Rhynchosporium species (e.g. Rhynchosporium secalis), Septoria species (e.g. Septoria apii or Septoria lycopersici), Stagonospora species (e.g. Stagonospora nodorum), Typhula species (e.g. Typhula incarnate), Venturia species (e.g. Venturia inaequalis),
root and stem diseases caused, for example, by Corticium species (e.g. Corticium graminearum), Fusarium species (e.g. Fusarium oxysporum), Gaeumannomyces species, (e.g. Gaeumannomyces graminis), Plasmodiophora species, (e.g. Plasmodiophora brassicae), Rhizoctonia species, (e.g. Rhizoctonia solani), Sarocladium species, (e.g. Sarocladium oryzae), Sclerotium species, (e.g. Sclerotium oryzae), Tapesia species, (e.g. Tapesia acuformis), Thielaviopsis species, (e.g. Thielaviopsis basicola);
ear and panicle diseases (including corn cobs) caused, for example, by Alternaria species, (e.g. Alternaria spp.), Aspergillus species (e.g. Aspergillus flavus), Cladosporium species (e.g. Cladosporium cladosporioides, Claviceps species (e.g. Claviceps purpurea), Fusarium species, (e.g. Fusarium culmorum), Gibberella species (e.g. Gibberella zeae), Monographella species, (e.g. Monographella nivalis), Stagnospora species, (e.g. Stagnospora nodorum);
diseases caused by smut fungi, for example Sphacelotheca species (e.g. Sphacelotheca reilana), Tilletia species (e.g. Tilletia caries or Tilletia controversa), Urocystis species (e.g. Urocystis occulta), Ustilago species (e.g. Ustilago nuda);
fruit rot caused, for example, by Aspergillus species (e.g. Aspergillus flavus), Botrytis species (e.g. Botrytis cinerea), Penicillium species (e.g. Penicillium expansum or Penicillium purpurogenum), Rhizopus species (e.g. Rhizopus stolonifer), Sclerotinia species (e.g. Sclerotinia sclerotiorum), Verticilium species (e.g. Verticilium alboatrum);
seed- and soil-borne rot and wilt diseases, and also diseases of seedlings, caused, for example, by Alternaria species (e.g. Alternaria brassicicola), Aphanomyces species (e.g. Aphanomyces euteiches), Ascochyta species (e.g. Ascochyta lentis), Aspergillus species (e.g. Aspergillus flavus), Cladosporium species (e.g. Cladosporium herbarum), Cochliobolus species (e.g. Cochliobolus sativus (conidial form: Drechslera, Bipolaris Syn: Helminthosporium)), Colletotrichum species (e.g. Colletotrichum coccodes), Fusarium species (e.g. Fusarium culmorum), Gibberella species (e.g. Gibberella zeae), Macrophomina species (e.g. Macrophomina phaseolina), Microdochium species (e.g. Microdochium nivale), Monographella species (e.g. Monographella nivalis), Penicillium species(e.g. Penicillium expansum), Phoma species (e.g. Phoma lingam), Phomopsis species (e.g. Phomopsis sojae), Phytophthora species (e.g. Phytophthora cactorum), Pyrenophora species (e.g. Pyrenophora graminea), Pyricularia species (e.g. Pyricularia oryzae), Pythium species (e.g. Pythium ultimum), Rhizoctonia species (e.g. Rhizoctonia solani), Rhizopus species (e.g. Rhizopus oryzae), Sclerotium species (e.g. Sclerotium rolfsii), Septoria species (e.g. Septoria nodorum), Typhula species (e.g. Typhula incarnate), Verticillium species (e.g. Verticillium dahlia);
cancers, galls and witches' broom caused, for example, by Nectria species (e.g. Nectria galligena); wilt diseases caused, for example, by Monilinia species (e.g. Monilinia laxa);
deformations of leaves, flowers and fruits caused, for example, by Exobasidium species (e.g. Exobasidium vexans), Taphrina species (e.g. Taphrina deformans);
degenerative diseases in woody plants, caused, for example, by Esca species (e.g. Phaeomoniella chlamydospora, Phaeoacremonium aleophilum or Fomitiporia mediterranea), Ganoderma species (e.g. Ganoderma boninense);
diseases of flowers and seeds caused, for example, by Botrytis species (e.g. Botrytis cinerea); diseases of plant tubers caused, for example, by Rhizoctonia species (e.g. Rhizoctonia solani), Helminthosporium species (e.g. Helminthosporium solani);
diseases caused by bacterial pathogens, for example Xanthomonas species (e.g. Xanthomonas campestris pv. Oryzae), Pseudomonas species (e.g. Pseudomonas syringae pv. Lachrymans), Erwinia species (e.g. Erwinia amylovora).

In particular, the compounds of formula (I) and compositions comprising thereof are efficient in controlling pythopathogenic fungi causing rust diseases.

### Seed Treatment

The method for controlling unwanted microorganisms may be used to protect seeds from phytopathogenic microorganisms, such as fungi.

The term “seed(s)” as used herein include dormant seed, primed seed, pregerminated seed and seed with emerged roots and leaves.

Thus, the present invention also relates to a method for protecting seeds and/or crops from unwanted microorganisms, such as bacteria or fungi, which comprises the step of treating the seeds with one or more compounds of formula (I) or a composition comprising thereof. The treatment of seeds with the compound(s) of formula (I) or or a composition comprising thereof not only protects the seeds from phytopathogenic microorganisms, but also the germinating plants, the emerged seedlings and the plants after emergence.

The seeds treatment may be performed prior to sowing, at the time of sowing or shortly thereafter.

When the seeds treatment is performed prior to sowing (e.g. so-called on-seed applications), the seeds treatment may be performed as follows: the seeds may be placed into a mixer with a desired amount of compound(s) of formula (I) or a composition comprising thereof (either as such or after dilution), the seeds and the compound(s) of formula (I) or the composition comprising thereof are mixed until a homogeneous distribution on seeds is achieved. If appropriate, the seeds may then be dried.

The invention also relates to seeds treated with one or more compounds of formula (I) or a composition comprising thereof. As said before, the use of treated seeds allows not only protecting the seeds before and after sowing from unwanted microorganisms, such as phytopathogenic fungi, but also allows protecting the germinating plants and young seedlings emerging from said treated seeds.

A large part of the damage to crop plants caused by harmful organisms is triggered by the infection of the seeds before sowing or after germination of the plant. This phase is particularly critical since the roots and shoots of the growing plant are particularly sensitive, and even small damage may result in the death of the plant.

Therefore, the present invention also relates to a method for protecting seeds, germinating plants and emerged seedlings, more generally to a method for protecting crop from phytopathogenic microorganisms, which comprises the step of using seeds treated by one or more compounds of formula (I) or a composition comprising thereof.

Preferably, the seed is treated in a state in which it is sufficiently stable for no damage to occur in the course of treatment. In general, seeds can be treated at any time between harvest and shortly after sowing. It is customary to use seeds which have been separated from the plant and freed from cobs, shells, stalks, coats, hairs or the flesh of the fruits. For example, it is possible to use seeds which have been harvested, cleaned and dried down to a moisture content of less than 15% by weight.

Alternatively, it is also possible to use seeds which, after drying, for example, have been treated with water and then dried again, or seeds just after priming, or seeds stored in primed conditions or pre-germinated seeds, or seeds sown on nursery trays, tapes or paper.

The amount of compound(s) of formula (I) or composition comprising thereof applied to the seed is typically such that the germination of the seed is not impaired, or that the resulting plant is not damaged. This must be ensured particularly in case the active ingredients would exhibit phytotoxic effects at certain application rates. The intrinsic phenotypes of transgenic plants should also be taken into consideration when determining the amount of compound(s) of formula (I) or composition comprising thereof to be applied to the seed in order to achieve optimum seed and germinating plant protection with a minimum amount of compound(s) of formula (I) or composition comprising thereof being employed.

As indicated above, the compounds of the formula (I) can be applied, as such, directly to the seeds, i.e. without the use of any other components and without having been diluted, or a composition comprising the compounds of formula (I) can be applied. Preferably, the compositions are applied to the seed in any suitable form. Examples of suitable formulations include solutions, emulsions, suspensions, powders, foams, slurries or combined with other coating compositions for seed, such as film forming materials, pelleting materials, fine iron or other metal powders, granules, coating material for inactivated seeds, and also ULV formulations. The formulations may be ready-to-use formulations or may be concentrates that need to be diluted prior to use.

These formulations are prepared in a known manner, for instance by mixing the active ingredient or mixture thereof with customary additives, for example customary extenders and solvents or diluents, dyes, wetting agents, dispersants, emulsifiers, antifoams, preservatives, secondary thickeners, adhesives, gibberellins, and also water.

These formulations are prepared in a known manner, by mixing the active ingredients or active ingredient combinations with customary additives, for example customary extenders and solvents or diluents, dyes, wetting agents, dispersants, emulsifiers, antifoams, preservatives, secondary thickeners, adhesives, gibberellins, and also water.

Useful dyes which may be present in the seed dressing formulations are all dyes which are customary for such purposes. It is possible to use either pigments, which are sparingly soluble in water, or dyes, which are soluble in water. Examples include the dyes known by the names Rhodamine B, C.I.

Pigment Red 112 and C.I. Solvent Red 1. Useful wetting agents which may be present in the seed dressing formulations are all substances which promote wetting and which are conventionally used for the formulation of active agrochemical ingredients. Usable with preference are alkylnaphthalenesulphonates, such as diisopropyl- or diisobutylnaphthalenesulphonates. Useful dispersants and/or emulsifiers which may be present in the seed dressing formulations are all nonionic, anionic and cationic dispersants conventionally used for the formulation of active agrochemical ingredients. Usable with preference are nonionic or anionic dispersants or mixtures of nonionic or anionic dispersants. Useful nonionic dispersants include especially ethylene oxide/propylene oxide block polymers, alkylphenol polyglycol ethers and tristryrylphenol polyglycol ether, and the phosphated or sulphated derivatives thereof. Suitable anionic dispersants are especially lignosulphonates, polyacrylic acid salts and arylsulphonate/formaldehyde condensates. Antifoams which may be present in the seed dressing formulations are all foam-inhibiting substances conventionally used for the formulation of active agrochemical ingredients. Silicone antifoams and magnesium stearate can be used with preference. Preservatives which may be present in the seed dressing formulations are all substances usable for such purposes in agrochemical compositions.

Examples include dichlorophene and benzyl alcohol hemiformal. Secondary thickeners which may be present in the seed dressing formulations are all substances usable for such purposes in agrochemical compositions. Preferred examples include cellulose derivatives, acrylic acid derivatives, xanthan, modified clays and finely divided silica. Adhesives which may be present in the seed dressing formulations are all customary binders usable in seed dressing products. Preferred examples include polyvinylpyrrolidone, polyvinyl acetate, polyvinyl alcohol and tylose.

The compounds of the formula (I) and the compositions comprising thereof are suitable for protecting seeds of any plant variety which is used in agriculture, in greenhouses, in forests or in horticulture.

More particularly, the seed is that of cereals (such as wheat, barley, rye, millet, triticale, and oats), oilseed rape, maize, cotton, soybean, rice, potatoes, sunflower, beans, coffee, peas, beet (e.g. sugar beet and fodder beet), peanut, vegetables (such as tomato, cucumber, onions and lettuce), lawns and ornamental plants. Of particular significance is the treatment of the seed of wheat, soybean, oilseed rape, maize and rice.

The compounds of formula (I) or the compositions comprising thereof can be used for treating transgenic seeds, in particular seeds of plants capable of expressing a protein which acts against pests, herbicidal damage or abiotic stress, thereby increasing the protective effect. Synergistic effects may also occur in interaction with the substances formed by expression.

### Application

The compound of the invention can be applied as such, or for example in the form of as ready-to-use solutions, emulsions, water- or oil-based suspensions, powders, wettable powders, pastes, soluble powders, dusts, soluble granules, granules for broadcasting, suspoemulsion concentrates, natural products impregnated with the compound of the invention, synthetic substances impregnated with the compound of the invention, fertilizers or microencapsulations in polymeric substances.

Application is accomplished in a customary manner, for example by watering, spraying, atomizing, broadcasting, dusting, foaming, spreading-on and the like. It is also possible to deploy the compound of the invention by the ultra-low volume method, via a drip irrigation system or drench application, to apply it in-furrow or to inject it into the soil stem or trunk. It is further possible to apply the compound of the invention by means of a wound seal, paint or other wound dressing.

The effective and plant-compatible amount of the compound of the invention which is applied to the plants, plant parts, fruits, seeds or soil will depend on various factors, such as the compound/composition employed, the subject of the treatment (plant, plant part, fruit, seed or soil), the type of treatment (dusting, spraying, seed dressing), the purpose of the treatment (curative and protective), the type of microorganisms, the development stage of the microorganisms, the sensitivity of the microorganisms, the crop growth stage and the environmental conditions.

When the compound of the invention is used as a fungicide, the application rates can vary within a relatively wide range, depending on the kind of application. For the treatment of plant parts, such as leaves, the application rate may range from 0.1 to 10 000 g/ha, preferably from 10 to 1000 g/ha, more preferably from 50 to 300 g/ha (in the case of application by watering or dripping, it is even possible to reduce the application rate, especially when inert substrates such as rockwool or perlite are used). For the treatment of seeds, the application rate may range from 0.1 to 200 g per 100 kg of seeds, preferably from 1 to 150 g per 100 kg of seeds, more preferably from 2.5 to 25 g per 100 kg of seeds, even more preferably from 2.5 to 12.5 g per 100 kg of seeds. For the treatment of soil, the application rate may range from 0.1 to 10 000 g/ha, preferably from 1 to 5000 g/ha.

These application rates are merely examples and are not intended to limit the scope of the present invention.

### Material Protection

The compound and the composition of the invention may also be used in the protection of materials, especially for the protection of industrial materials against attack and destruction by unwanted microorganisms.

In addition, the compound and the composition of the invention may be used as antifouling compositions, alone or in combinations with other active ingredients.

Industrial materials in the present context are understood to mean inanimate materials which have been prepared for use in industry. For example, industrial materials which are to be protected from microbial alteration or destruction may be adhesives, glues, paper, wallpaper and board/cardboard, textiles, carpets, leather, wood, fibers and tissues, paints and plastic articles, cooling lubricants and other materials which can be infected with or destroyed by microorganisms. Parts of production plants and buildings, for example cooling-water circuits, cooling and heating systems and ventilation and air-conditioning units, which may be impaired by the proliferation of microorganisms may also be mentioned within the scope of the materials to be protected. Industrial materials within the scope of the present invention preferably include adhesives, sizes, paper and card, leather, wood, paints, cooling lubricants and heat transfer fluids, more preferably wood.

The compound and the composition of the invention may prevent adverse effects, such as rotting, decay, discoloration, decoloration or formation of mould.

In the case of treatment of wood the compound and the composition of the invention may also be used against fungal diseases liable to grow on or inside timber.

Timber means all types of species of wood, and all types of working of this wood intended for construction, for example solid wood, high-density wood, laminated wood, and plywood. In addition, the compound and the composition of the invention may be used to protect objects which come into contact with saltwater or brackish water, especially hulls, screens, nets, buildings, moorings and signalling systems, from fouling.

The compound and the composition of the invention may also be employed for protecting storage goods. Storage goods are understood to mean natural substances of vegetable or animal origin or processed products thereof which are of natural origin, and for which long-term protection is desired. Storage goods of vegetable origin, for example plants or plant parts, such as stems, leaves, tubers, seeds, fruits, grains, may be protected freshly harvested or after processing by (pre)drying, moistening, comminuting, grinding, pressing or roasting. Storage goods also include timber, both unprocessed, such as construction timber, electricity poles and barriers, or in the form of finished products, such as furniture. Storage goods of animal origin are, for example, hides, leather, furs and hairs. The compound and the composition of the invention may prevent adverse effects, such as rotting, decay, discoloration, decoloration or formation of mould.

Microorganisms capable of degrading or altering industrial materials include, for example, bacteria, fungi, yeasts, algae and slime organisms. The compound and the composition of the invention preferably act against fungi, especially moulds, wood-discoloring and wood-destroying fungi (Ascomycetes, Basidiomycetes, Deuteromycetes and Zygomycetes), and against slime organisms and algae. Examples include microorganisms of the following genera: Alternaria, such as Alternaria tenuis; Aspergillus, such as Aspergillus niger Chaetomium, such as Chaetomium globosum; Coniophora, such as Coniophora puetana; Lentinus, such as Lentinus tigrinus; Penicillium, such as Penicillium glaucum; Polyporus, such as Polyporus versicolor; Aureobasidium, such as Aureobasidium pullulans; Sclerophoma, such as Sclerophoma pityophila; Trichoderma, such as Trichoderma viride; Ophiostoma spp., Ceratocystis spp., Humicola spp., Petriella spp., Trichurus spp., Coriolus spp., Gloeophyllum spp., Pleurotus spp., Poria spp., Serpula spp. and Tyromyces spp., Cladosporium spp., Paecilomyces spp. Mucor spp., Escherichia, such as Escherichia coli; Pseudomonas, such as Pseudomonas aeruginosa; Staphylococcus, such as Staphylococcus aureus, Candida spp. and Saccharomyces spp., such as Saccharomyces cerevisae.

### General Synthetic Routes to the Compounds of General Formula I

The present invention also relates to processes for the preparation of compounds of formula (I). Unless indicated otherwise, n and the radicals A, R1, R2, R3 and X have the meanings given above for the compounds of formula (I). These definitions apply not only to the end products of the formula (I) but likewise to all intermediates.

Compounds of formula (I) can be prepared, according to process P1, by reacting chloroxymes of formula (II) with imines of formula (II) in a suitable solvent such as diethylether or tetrahydrofuran optionally in presence of a base such as triethylamine, preferably at room temperature, as previously described in WO2003028729 or Synthesis, 2011, 21, 9426.

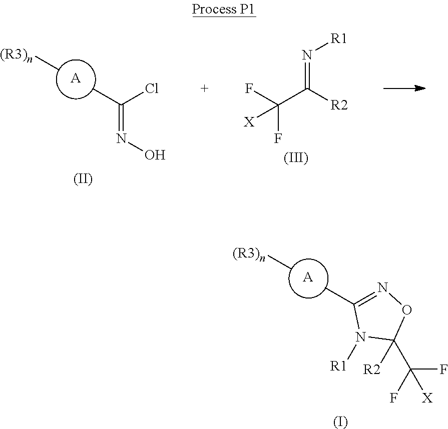

Compounds of formula (II) can be commercially available or may be prepared starting from readily available compounds according to known procedures.

Compounds of formula (II) can be commercially available or may be prepared starting from readily available compounds according to known procedures.

Alternatively, compounds of formula (I-a) can be prepared, according to process P2, by reacting oxadiazoles of formula (IV) with a reducing agent such as sodium borohydride in a suitable solvent such as methanol, preferably at 0° C., as previously described in WO001007436.

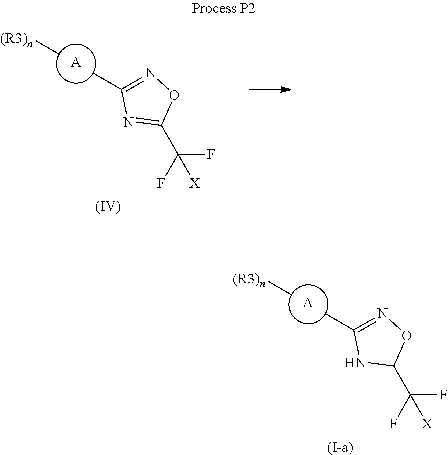

Compounds of formula (IV) can be prepared, according to process P3, by reacting amidoximes of formula (V) with difluorohaloalkylacetic anhydride or difluorohaloalkylacetyl chloride in a suitable solvent such as tetrahydrofuran or dichloromethane optionally in presence of a base such as triethylamine or pyridine, preferably at room temperature, as previously described in WO2013080120.

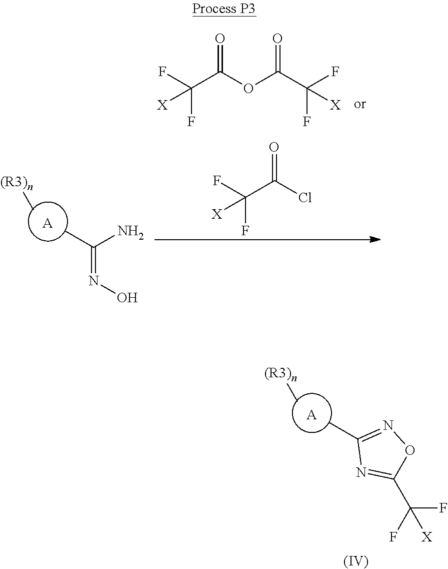

Amidoximes of formula (V) can be prepared according to known procedures (see for examples WO2013080120), as shown in process P4 by treating nitriles of formula (VI) with hydroxylamine (or its hydrochloride salt) in the presence of a base such as triethylamine in a solvent such as ethanol.

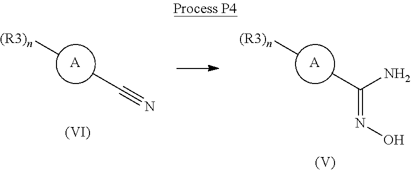

Compounds of formula (VI) can be commercially available or may be prepared starting from readily available compounds according to known procedures.

Alternatively compounds of formula (VI) can be prepared, according to process P5, from compounds of formula (VII), wherein LG1 is a leaving group as for example bromide with a suitable cyanide reagent such as for example zinc cyanide in presence of a palladium (0) source such as tetrakis(triphenylphosphine)palladium (0) in a solvent such as N,N-dimethylformamide as described for example in ACS Medicinal Chemistry Letters, 8(9), 919-924, 2017.

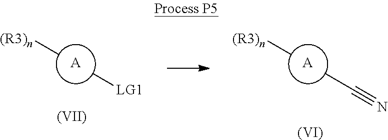

Compounds of formula (VII) can be commercially available or may be prepared starting from readily available compounds according to known procedures.

According to the invention, processes P1 to P5 can be performed if appropriate in the presence of a solvent and if appropriate in the presence of a base.

Suitable solvents for carrying out processes P1 to P5 according to the invention are customary inert organic solvents. Preference is given to using optionally halogenated aliphatic, alicyclic or aromatic hydrocarbons, such as petroleum ether, hexane, heptane, cyclohexane, methylcyclohexane, benzene, toluene, xylene or decalin; chlorobenzene, dichlorobenzene, dichloromethane, chloroform, carbon tetrachloride, dichlorethane or trichlorethane; ethers, such as diethyl ether, diisopropyl ether, methyl tert-butyl ether, methyl tert-amyl ether, dioxane, tetrahydrofuran, 1,2-dimethoxyethane, 1,2-diethoxyethane or anisole; nitriles, such as acetonitrile, propionitrile, n- or iso-butyronitrile or benzonitrile; amides, such as N,N-dimethylformamide, N,N-dimethylacetamide, N-methylformanilide, N-methylpyrrolidone or hexamethylphosphoric triamide; esters, such as methyl acetate or ethyl acetate, sulfoxides, such as dimethyl sulfoxide or sulfones, such as sulfolane.

Suitable bases for carrying out processes P1 to P5 according to the invention are inorganic and organic bases which are customary for such reactions. Preference is given to using alkaline earth metal, alkali metal hydride, alkali metal hydroxides or alkali metal alkoxides, such as sodium hydroxide, sodium hydride, calcium hydroxide, potassium hydroxide, potassium tert-butoxide or other ammonium hydroxide, alkali metal carbonates, such as sodium carbonate, potassium carbonate, potassium bicarbonate, sodium bicarbonate, cesium carbonate, alkali metal or alkaline earth metal acetates, such as sodium acetate, potassium acetate, calcium acetate and also tertiary amines, such as triethylamine, diisopropylethylamine, tributylamine, N,N-dimethylaniline, pyridine, N-methylpiperidine, N,N-dimethylaminopyridine, 1,4-diazabicyclo[2.2.2]octane (DABCO), 1,5-diazabicyclo[4.3.0]non-5-ene (DBN) or 1,8-diazabicyclo[5.4.0]undec-7-ene (DBU).

When carrying out processes P1 to P5, according to the invention, the reaction temperature can independently be varied within a relatively wide range. Generally, processes according to the invention are carried out at temperatures between −20° C. and 160° C.

Processes P1 to P5 according to the invention are generally independently carried out under atmospheric pressure. However, it is also possible to operate under elevated or reduced pressure.

Work-up is carried out by customary methods. Generally, the reaction mixture is treated with water and the organic phase is separated off and, after drying, concentrated under reduced pressure. If appropriate, the remaining residue can be freed by customary methods, such as chromatography or recrystallization, from any impurities that can still be present.

Compounds according to the invention can be prepared according to the above described processes. It will nevertheless be understood that, on the basis of his general knowledge and of available publications, the skilled worker will be able to adapt these processes according to the specifics of each of the compounds according to the invention that is desired to be synthesized.

Aspects of the present teaching may be further understood in light of the following examples, which should not be construed as limiting the scope of the present teaching in any way.

### Examples

The following examples illustrate in a non-limiting manner the preparation and efficacy of the compounds of formula (I) according to the invention.

### Synthesis of Intermediates of Formula IV

### 4-[5-(Trifluoromethyl)-1,2,4-oxadiazol-3-yl]phenyl dimethylcarbamate (Intermediate IV-05)

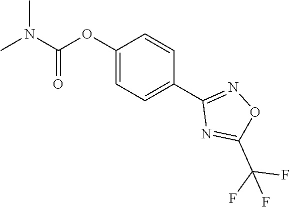 [SMILES: CN(C)C(=O)Oc1ccc(-c2noc(C(F)(F)F)n2)cc1]

To a suspension of 4-[5-(trifluoromethyl)-1,2,4-oxadiazol-3-yl]phenol (0.8 g, 3.5 mmol) in acetonitrile (15 ml), were added potassium carbonate (0.72 g, 5.2 mmol) and dimethylcarbamyl chloride (0.49 ml, 5.2 mmol). The mixture was refluxed for 3 h. The solvent was then evaporated and the residue was dissolved in water and extracted three times with ethyl acetate. The combined organic fractions were washed with a sodium hydroxide 1M solution, dried over magnesium sulfate and concentrated over reduced pressure. The residue was purified by column chromatography to afford 4-[5-(trifluoromethyl)-1,2,4-oxadiazol-3-yl]phenyl dimethylcarbamate (0.96 g, 89% yield) as a white solid.

### 5-[5-(Trifluoromethyl)-1,2,4-oxadiazol-3-yl]-1H-indazole

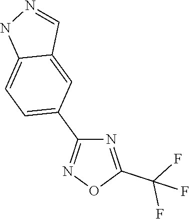

To a suspension of 1H-indazole-5-carbonitrile (1.0 g, 7.2 mmol) in ethanol (25 ml) was added hydroxylamine hydrochloride (1.0 g, 14 mmol), followed by triethylamine (4.5 ml, 33 mmol). The orange solution was stirred at room temperature for 18 h. The reaction mixture was concentrated under reduced pressure and the residue was suspended in tetrahydrofuran (30 ml). Trifluoroacetic anhydride (1.9 ml, 14 mmol) was then added and the reaction mixture was stirred at room temperature for 5 h. Trifluoroacetic anhydride (1.0 ml, 6.8 mmol) was then added and the reaction mixture was stirred at room temperature for 3 h. The solvent was evaporated under reduced pressure and the residue was diluted in ethyl acetate and washed with water, with a saturated sodium hydrogen carbonate solution and then with brine. The organic layer was dried over magnesium sulfate and concentrated under reduced pressure to afford 5-[5-(trifluoromethyl)-1,2,4-oxadiazol-3-yl]-1H-indazole (0.62 g, 34% yield) as a yellow solid.

MS (ESI): 255 ([M+H]+)

### 1-Benzyl-5-[5-(trifluoromethyl)-1,2,4-oxadiazol-3-yl]-1H-indazole (Intermediate IV-17)

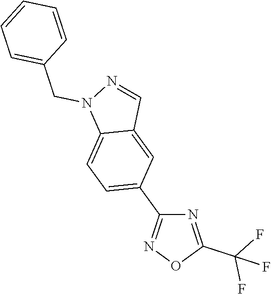 [SMILES: FC(F)(F)c1nc(-c2ccc3c(cnn3Cc3ccccc3)c2)no1]

To a solution of 5-[5-(trifluoromethyl)-1,2,4-oxadiazol-3-yl]-1H-indazole (0.12 g, 0.47 mmol) in N,N-dimethylformamide (3 ml) was added potassium carbonate (0.16 g, 1.2 mmol) and the mixture was stirred at room temperature for 30 minutes. Benzyl bromide (62 μl, 0.51 mmol) was then added and the mixture was stirred at room temperature for 2 days. Water was then added and the reaction mixture was extracted twice with ethyl acetate. The organic layers were combined, washed with brine, dried over magnesium sulfate and concentrated under vacuum. The residue was purified by column chromatography to afford the title compound 1-benzyl-5-[5-(trifluoromethyl)-1,2,4-oxadiazol-3-yl]-1H-indazole (75 mg, 46% yield) as a yellow solid and its regioisomer 2-(2-methoxyethyl)-5-[5-(trifluoromethyl)-1,2,4-oxadiazol-3-yl]-2H-indazole (40 mg, 24% yield) as a yellow solid.

MS (ESI): 345 ([M+H]+)

### 2-Benzyl-5-[5-(trifluoromethyl)-1,2,4-oxadiazol-3-yl]-1H-indazole (Intermediate IV-15)

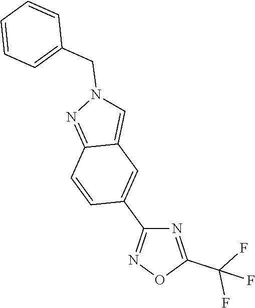 [SMILES: FC(F)(F)c1nc(-c2ccc3nn(Cc4ccccc4)cc3c2)no1]

MS (ESI): 345 ([M+H]+)

### Synthesis of Compounds of Formula (I)

### 4-[5-(Trifluoromethyl)-4,5-dihydro-1,2,4-oxadiazol-3-yl]phenyl dimethylcarbamate (Compound I-03)

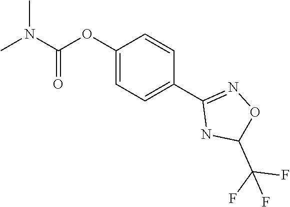

To a suspension of 4-[5-(trifluoromethyl)-1,2,4-oxadiazol-3-yl]phenyl dimethylcarbamate (120 mg, 0.39 mmol) in methanol (15 ml) was added sodium borohydride (72 mg, 1.9 mmol) at 0° C. The reaction was stirred at 0° C. for 30 min. The solvent was evaporated under reduced pressure and the residue was diluted in water and extracted three times with ethyl acetate. The combined organic fractions were washed with brine, dried over magnesium sulfate and concentrated over reduced pressure. The residue was purified by column chromatography to afford 4-[5-(trifluoromethyl)-4,5-dihydro-1,2,4-oxadiazol-3-yl]phenyl dimethylcarbamate (29 mg, 22% yield).

MS (ESI): 304 ([M+H]+)

### 4-[Acetyl-5-(trifluoromethyl)-4,5-dihydro-1,2,4-oxadiazol-3-yl]phenyl dimethylcarbamate (Compound I-16)

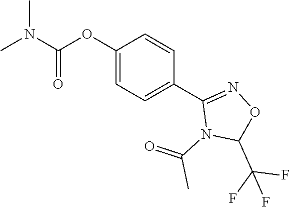 [SMILES: CC(=O)N1C(c2ccc(OC(=O)N(C)C)cc2)=NOC1C(F)(F)F]

To a solution of 4-[5-(trifluoromethyl)-4,5-dihydro-1,2,4-oxadiazol-3-yl]phenyl dimethylcarbamate (Compound I-03) (60 mg, 0.19 mmol) in dichloromethane (4 ml) were added triethylamine (55 μl, 0.39 mmol) and acetic anhydride (24 μl, 0.25 mmol). The reaction was stirred at 40° C. for 90 min. The reaction mixture was then diluted in dichloromethane, washed twice with water, dried over magnesium sulfate and concentrated over reduced pressure. The residue was purified by preparative HPLC to afford 4-[acetyl-5-(trifluoromethyl)-4,5-dihydro-1,2,4-oxadiazol-3-yl]phenyl dimethylcarbamate (41 mg, 59% yield).

### 1-Benzyl-5-[(trifluoromethyl)-4,5-dihydro-1,2,4-oxadiazol-3-yl]-2H-indazole (Compound I-23)

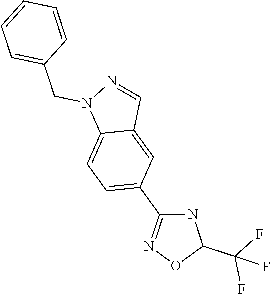

To a solution of 1-benzyl-5-[5-(trifluoromethyl)-1,2,4-oxadiazol-3-yl]-1H-indazole (Intermediate IV-17) (0.26 g, 0.74 mmol) in methanol (4 ml) was added borohydride polymer-supported (2.5 mmol BH₄⁻/g resin) (0.6 g, 1.5 mmol) and the mixture was shaked at room temperature for 22 h. The reaction mixture was filtered, washed with methanol and concentrated under vacuum. The residue was purified by column chromatography to afford the title compound 1-benzyl-5-[(trifluoromethyl)-4,5-dihydro-1,2,4-oxadiazol-3-yl]-2H-indazole (0.12 g, 45% yield) as a white solid.

MS (ESI): 347 ([M+H]+)

### 2-Benzyl-5-[(trifluoromethyl)-4,5-dihydro-1,2,4-oxadiazol-3-yl]-2H-indazole (Compound I-24)

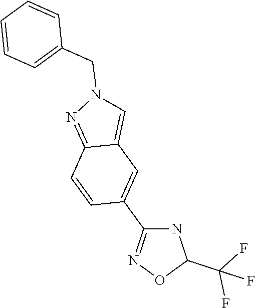

To a solution of 2-benzyl-5-[5-(trifluoromethyl)-1,2,4-oxadiazol-3-yl]-1H-indazole (Intermediate IV-15) (50 mg, 0.14 mmol) in methanol (4 ml) was added borohydride polymer-supported (2.5 mmol BH₄⁻/g resin) (0.11 g, 0.29 mmol) and the mixture was shaked at room temperature for 22 h. The reaction mixture was filtered, washed with methanol and concentrated under vacuum. The residue was purified by column chromatography to afford the title compound 2-benzyl-5-[(trifluoromethyl)-4,5-dihydro-1,2,4-oxadiazol-3-yl]-2H-indazole (40 mg, 78% yield) as a white solid.

MS (ESI): 347 ([M+H]+)

The compounds as shown in table 1 below were prepared in analogy with the examples provided above.

TABLE 1
Compounds according to formula (I)

| | | | | | (I) |
|---|---|---|---|---|---|
| 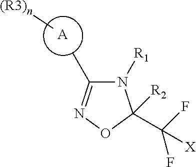 | | | | | |
| Ex N° | R₁ | R₂ | 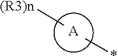 | X | Log P |
| I-01 | H | H | 4-[(methoxycarbonyl)amino]phenyl | F | 1.99^{[a]} |
| I-02 | H | H | 4-[[(cyclopropylcarbonyl)amino]methyl]phenyl | F | 1.86^{[a]} |
| I-03 | H | H | 4-(dimethylaminocarbonyl)oxyphenyl | F | 2.19^{[a]} |
| I-04 | H | H | 4-[[(cyclopropylcarbonyl)-methoxyamino]methyl]phenyl | F | 2.42^{[a]} |
| I-05 | H | H | 3-methyl-3H-benzimidazol-5-yl | F | 0.95^{[a]} |
| I-06 | H | H | 4-methylphenyl | F | 2.59^{[a]} |
| I-07 | H | H | 1-methyl-1H-benzimidazol-5-yl | F | 0.99^{[a]} |
| I-08 | H | H | 4-[[acetyl(methoxy)amino]methyl]phenyl | F | 1.98^{[a]} |
| I-09 | H | H | 3H-benzimidazol-5-yl | F | 0.80^{[a]} |
| I-10 | H | H | 4-[(2,4-difluoroanilino)carbonyl]phenyl | F | 2.68^{[a]} |
| I-11 | H | H | 4-[[(1-methylcyclopropyl)amino]carbonyl]phenyl | F | 2.01^{[a]} |
| I-12 | H | H | 4-(methylcarbamoyl)phenyl | F | 1.47^{[a]} |
| I-13 | H | H | 4-[(cyclopropylmethylamino)carbonyl]phenyl | F | 2.11^{[a]} |
| I-14 | H | H | 4-(methylcarbamothioyl)phenyl | F | 2.30^{[a]} |
| I-15 | H | H | 4-(cyclopropylmethylcarbamothioyl)phenyl | F | 2.92^{[a]} |
| I-16 | acetyl | H | 4-(dimethylaminocarbonyl)oxyphenyl | F | 2.62^{[a]} |
| I-17 | H | H | 4-[(N-methylanilino)carbonyl]oxyphenyl | F | 3.06^{[a]} |
| I-18 | H | H | 4-[[ethyl(methyl)amino]carbonyl]oxyphenyl | F | 2.66^{[a]} |
| I-19 | H | H | 1-(2-methoxyethyl)-1H-indazol-6-yl | F | 2.19^{[a]} |
| I-20 | H | H | 2-(methoxycarbonyl)-1,2,3,4-tetrahydroisoquinolin-7-yl | Cl | 2.50^{[a]} |
| I-21 | H | H | 1-benzyl-6-fluoro-1H-indazol-5-yl | F | 3.02^{[a]} |
| I-22 | H | H | 2-benzyl-6-fluoro-2H-indazol-5-yl | F | 2.75^{[a]} |
| I-23 | H | H | 1-benzyl-1H-indazol-5-yl | F | 3.00^{[a]} |
| I-24 | H | H | 2-benzyl-2H-indazol-5-yl | F | 2.69^{[a]} |
| I-25 | H | H | 4-[[isopropyl(methyl)amino]carbonyl]oxyphenyl | F | 2.86^{[a]} |
| I-26 | H | H | 4-[[(methoxycarbonyl)amino]methyl]phenyl | F | 1.91^{[a]} |
| I-27 | H | H | 4-[[[(4-fluorophenoxy)carbonyl]-methoxyamino]methyl]phenyl | F | 3.21^{[a]} |
| I-28 | H | H | 4-(hydroxyiminomethyl)phenyl | F | 1.88^{[a]} |
| I-29 | H | H | 4-[(2S)-2-(tert-butoxycarbonyl)-5-oxopyrrolidin-1-yl]phenyl | F | 2.78^{[a]} |
| I-30 | H | H | 4-[(2-methoxyethylamino)carbonyl]phenyl | F | 1.63^{[a]} |
| I-31 | H | H | 1-propionyl-1,2,3,4-tetrahydroquinolin-6-yl | F | 2.40^{[a]} |
| I-32 | H | H | 6-(d imethylaminocarbonyl)oxynaphthalen-2-yl | F | 2.84^{a]} |
| I-33 | H | H | 1H-indo1-6-yl | CI | 2.30^{[a]} |
| I-34 | H | H | 4-methylsulfanylphenyl | F | 2.70^{[a]} |
| I-35 | H | H | 1-oxo-1,2-dihydroisoquinolin-6-yl | F | 1.63^{[a]} |
| I-36 | H | H | 1H-indo1-6-yl | F | 2.19^{[a]} |
| I-37 | H | H | 4-methylsulfanylphenyl | Cl | 2.82^{[a]} |
| I-38 | H | H | 4-(dimethylaminomethyleneamino)phenyl | F | 0.84^{[a]} |
| I-39 | H | H | 3-benzyl-3H-benzimidazol-5-yl | F | 2.01^{[a]} |
| I-40 | H | H | 4-[hydroxyimino(methylsulfanyl)methyl]phenyl | F | 2.08^{[a]} |
| I-41 | H | H | 4-[[cyclohexyl(methyl)amino]carbonyl]oxyphenyl | F | 3.71^{[a]} |
| I-42 | H | H | 2-propionyl-1,2,3,4-tetrahydroisoquinolin-7-yl | Cl | 2.23^{[a]} |
| I-43 | H | H | 4-[1-(acetylamino)cyclopropyl]phenyl | F | 1.65^{[a]} |
| I-44 | H | H | 4-[[methyl-[(4-thiophen-2-yloxan-4-yl)methyl]amino] | F | 2.70^{[a]} |
| | | | carbonyl]phenyl | | |
| I-45 | H | H | 4-(propionylamino)phenyl | F | 1.91^{[a]} |
| I-46 | H | H | 1-(methoxycarbonyl)-1,2,3,4-tetrahydroguinolin-6-yl | Cl | 2.77^{[a]} |
| I-47 | H | H | 4-[[2(4-chlorophenoxy)-2-methylpropanoyl]amino]phenyl | F | 3.81^{[a]} |
| I-48 | H | H | 4-[[(1-methylcyclopropyl)carbonyl]amino]phenyl | F | 2.36^{[a]} |
| I-49 | H | H | 4-[(N-methylanilino)carbonyl]phenyl | F | 2.57^{[a]} |
| I-50 | H | H | 4-(methoxycarbonyl)phenyl | F | 2.46^{[a]} |
| I-51 | H | H | 4-[3-[(methoxycarbonyl)amino]phenyl]phenyl | F | 2.91^{[a]} |
| I-52 | H | H | 4-[2-[(methoxycarbonyl)amino]phenyl]phenyl | F | 2.80^{[a]} |
| I-53 | H | H | 4-[(4-methoxyphenyl)thio]phenyl | F | 3.81^{[a]} |
| I-54 | H | H | 4-(4-methoxyphenyl)sulfinylphenyl | F | 2.39^{[a]} |
| I-55 | H | H | 4-[(2,2-dimethylhydrazino)carbonyl]phenyl | F | 1.31^{[a]} |
| I-56 | H | H | 4-(3-ethyl-1,2,4-oxadiazol-5-yl)phenyl | F | 2.94^{[a]} |
| I-57 | H | H | 4-[1,1-difluoro-2-oxo-2-(prop-2-yn-1-ylamino)ethyl]phenyl | F | 2.36^{[a]} |
| I-58 | H | H | 4(4-methoxyphenyl)sulfonylphenyl | F | 2.89^{[a]} |

The intermediates as shown in table 2 below were prepared in analogy with the examples provided above.

TABLE 2
Intermediates according to formula (IV)

| | | | (IV) |
|---|---|---|---|
| 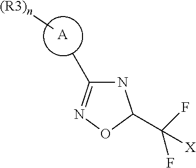 | | | |
| Ex N° | 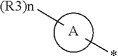 | X | LogP |
| IV-01 | 4-[[(cyclopropylcarbonyl)- | F | 3.53^{[a]} |
| | methoxyamino]methyl]phenyl | | |
| IV-02 | 4-methylphenyl | F | 4.33^{[a]} |
| IV-03 | 3-methyl-3H-benzimidazol-5-yl | F | 1.67^{[a]} |
| IV-04 | 4-[[acetyl(methoxy)amino]methyl]phenyl | F | 2.96^{[a]} |
| IV-05 | 4-(dimethylaminocarbonyl)oxyphenyl | F | 3.37^{[a]} |
| IV-06 | 1-methyl-1H-benzimidazol-5-yl | F | 1.70^{[a]} |
| IV-07 | 4-[[(cyclopropylcarbonyl)amino]methyl]phenyl | F | 2.71^{[a]} |
| IV-08 | 4-[(methoxycarbonyl)amino]phenyl | F | 3.06^{[a]} |
| IV-09 | 3H-benzimidazol-5-yl | F | 1.46^{[a]} |
| IV-10 | 4-(methoxycarbonyl)phenyl | F | |
| IV-11 | 4-[3-[(methoxycarbonyl)amino]phenyl]phenyl | F | 4.20^{[a]} |
| IV-12 | 4-[2-[(methoxycarbonyl)amino]phenyl]phenyl | F | 3.94^{[a]} |
| IV-13 | 6-(dimethylaminocarbonyl)oxynaphthalen-2-yl | F | 4.18^{[a]} |
| IV-14 | 1-(2-methoxyethyl)-1H-indazol-6-yl | F | 3.35^{[a]} |
| IV-15 | 2-benzyl-2H-indazol-5-yl | F | 4.03^{[a]} |
| IV-16 | 1-propionyl-1,2,3,4-tetrahydroguinolin-6-yl | F | 3.68^{[a]} |
| IV-17 | 1-benzyl-1H-indazol-5-yl | F | 4.39^{[a]} |
| IV-18 | 4-(dimethylaminomethyleneamino)phenyl | F | 1.79^{[b]} |
| IV-19 | 1H-indo1-6-yl | F | 3.50^{[a]} |
| IV-20 | 3-benzyl-3H-benzimidazol-5-yl | F | 3.06^{[a]} |
| IV-21 | 1-benzyl-6-fluoro-1H-indazol-5-yl | F | 4.28^{[a]} |
| IV-22 | 2-benzyl-6-fluoro-2H-indazol-5-yl | F | 3.92^{[a]} |
| IV-23 | 4-[[cyclohexyl(methyl)amino]carbonyl]oxyphenyl | F | 5.17^{[a]} |
| IV-24 | 1-(methoxycarbonyl)-1,2,3,4- | Cl | 4.27^{[a]} |
| | tetrahydroquinolin-6-yl | | |
| IV-25 | 2-(methoxycarbonyl)-1,2,3,4- | Cl | 3.83^{[a]} |
| | tetrahydroisoguinolin-7-yl | | |
| IV-26 | 2-propionyl-1,2,3,4- | Cl | 3.31^{[a]} |
| | tetrahydroisoguinolin-7-yl | | |
| IV-27 | 1H-indo1-6-yl | Cl | 3.68^{[a]} |
| IV-28 | 4-methylsulfanylphenyl | Cl | 4.61^{[a]} |
| IV-29 | 4-[[2-(4-chlorophenoxy)-2- | F | 5.11^{[a]} |
| | methylpropanoyl]amino]phenyl | | |
| IV-30 | 4-[[methyl-[(4-thiophen-2-yloxan-4-yl) | F | 3.71^{[a]} |
| | methyl]amino]carbonyl]phenyl | | |
| IV-31 | 1-oxo-1,2-dihydroisoquinolin-6-yl | F | 2.39^{[a]} |
| IV-32 | 4-[(N-methylanilino)carbonyl]oxyphenyl | F | 4.27^{[a]} |
| IV-33 | 4-[(2S)-2-(tert-butoxycarbonyl)-5- | F | 3.95^{[a]} |
| | oxopyrrolidin-1-yl]phenyl | | |
| IV-34 | 4-(4-methoxyphenyl)sulfonylphenyl | F | 3.72^{[a]} |
| IV-35 | 4-[[[(4-fluorophenoxy)carbonyl]- | F | 4.40^{[a]} |
| | methoxyamino]methyl]phenyl | | |
| IV-36 | 4-(4-methoxyphenyl)sulfinylphenyl | F | 3.26^{[a]} |
| IV-37 | 4-[(N-methylanilino)carbonyl]phenyl | F | 3.50^{[a]} |
| IV-38 | 4-[(cyclopropylmethylamino)carbonyl]phenyl | F | 3.04^{[a]} |
| IV-39 | 4-[[(1-methylcyclopropyl)carbonyl]amino]phenyl | F | 3.60^{[a]} |
| IV-40 | 4-(methylcarbamoyl)phenyl | F | 2.21^{[a]} |
| IV-41 | 4-(propionylamino)phenyl | F | 3.00^{[a]} |
| IV-42 | 4-[(4-methoxyphenyl)thio]phenyl | F | 5.37^{[a]} |
| IV-43 | 4-[(2-methoxyethylamino)carbonyl]phenyl | F | 2.42^{[a]} |
| IV-44 | 4-[[ethyl(methyl)amino]carbonyl]oxyphenyl | F | 3.79^{[a]} |
| IV-45 | 4-[1,1-difluoro-2-oxo-2-(prop-2- | F | |
| | yn-1-ylamino)ethyl]phenyl | | |
| IV-46 | 4-(3-ethyl-1,2,4-oxadiazol-5-yl) phenyl | F | |
| IV-47 | 4-[(2,2-dimethylhydrazino)carbonyl]phenyl | F | |
| IV-48 | 4-[1-(acetylamino)cyclopropyl]phenyl | F | |
| IV-49 | 4-[(2,4-difluoroanilino)carbonyl]phenyl | F | 3.72^{[a]} |
| IV-50 | 4-[[(1-methylcyclopropyl)amino]carbonyl]phenyl | F | 2.92^{[a]} |
| IV-51 | 4-(methylcarbamothioyl)phenyl | F | 3.19^{[a]} |
| IV-52 | 4-methylsulfanylphenyl | F | 4.24^{[a]} |
| IV-53 | 4-(hydroxyiminomethyl)phenyl | F | 2.90^{[a]} |
| IV-54 | 4-[hydroxyimino(methylsulfanyl)methyl]phenyl | F | 3.08^{[a]} |
| IV-55 | 4-(cyclopropylmethylcarbamothioyl)phenyl | F | 4.03^{[a]} |
| IV-56 | 4-[[isopropyl(methyl)amino]carbonyl]oxyphenyl | F | 4.18^{[a]} |

Measurement of Log P values was performed according to EEC directive 79/831 Annex V.A8 by HPLC (High Performance Liquid Chromatography) on reversed phase columns with the following methods:
[^{a}] Log P value is determined by measurement of LC-UV, in an acidic range, with 0.1% formic acid in water and acetonitrile as eluent (linear gradient from 10% acetonitrile to 95% acetonitrile).
[^{b}] Log P value is determined by measurement of LC-UV, in a neutral range, with 0.001 molar ammonium acetate solution in water and acetonitrile as eluent (linear gradient from 10% acetonitrile to 95% acetonitrile).
[^{c}] Log P value is determined by measurement of LC-UV, in an acidic range, with 0.1% phosphoric acid and acetonitrile as eluent (linear gradient from 10% acetonitrile to 95% acetonitrile).

If more than one Log P value is available within the same method, all the values are given and separated by “+”.

Calibration was done with straight-chain alkan2-ones (with 3 to 16 carbon atoms) with known Log P values (measurement of Log P values using retention times with linear interpolation between successive alkanones). Lambda-max-values were determined using UV-spectra from 200 nm to 400 nm and the peak values of the chromatographic signals.

### NMR-Peak Lists

1H-NMR data of selected examples are written in form of 1H-NMR-peak lists. To each signal peak are listed the δ-value in ppm and the signal intensity in round brackets. Between the δ-value—signal intensity pairs are semicolons as delimiters.

The peak list of an example has therefore the form:

δ₁ (intensity₁); δ₂ (intensity₂); . . . ; δᵢ (intensity); . . . ; δₙ (intensityₙ)

Intensity of sharp signals correlates with the height of the signals in a printed example of a NMR spectrum in cm and shows the real relations of signal intensities. From broad signals several peaks or the middle of the signal and their relative intensity in comparison to the most intensive signal in the spectrum can be shown.

For calibrating chemical shift for 1H spectra, we use tetramethylsilane and/or the chemical shift of the solvent used, especially in the case of spectra measured in DMSO. Therefore in NMR peak lists, tetramethylsilane peak can occur but not necessarily.

The 1H-NMR peak lists are similar to classical 1H-NMR prints and contains therefore usually all peaks, which are listed at classical NMR-interpretation.

Additionally they can show like classical 1H-NMR prints signals of solvents, stereoisomers of the target compounds, which are also object of the invention, and/or peaks of impurities.

To show compound signals in the delta-range of solvents and/or water the usual peaks of solvents, for example peaks of DMSO in DMSO-D₆ and the peak of water are shown in our 1H-NMR peak lists and have usually on average a high intensity.

The peaks of stereoisomers of the target compounds and/or peaks of impurities have usually on average a lower intensity than the peaks of target compounds (for example with a purity >90%).

Such stereoisomers and/or impurities can be typical for the specific preparation process. Therefore their peaks can help to recognize the reproduction of our preparation process via “side-products-fingerprints”.

An expert, who calculates the peaks of the target compounds with known methods (MestreC, ACD-simulation, but also with empirically evaluated expectation values) can isolate the peaks of the target compounds as needed optionally using additional intensity filters. This isolation would be similar to relevant peak picking at classical 1H-NMR interpretation.

Further details of NMR-data description with peak lists you find in the publication “Citation of NMR Peaklist Data within Patent Applications” of the Research Disclosure Database Number 564025.

I-01: ¹H-NMR(499.9 MHz, d₆-DMSO):
δ = 7.6426 (3.3); 7.6250 (5.3); 7.5718 (4.4); 7.5543 (2.8); 6.2412 (0.4); 6.2321 (1.4); 6.2225 (1.5); 6.2128 (0.6); 3.7089 (0.5);
3.7004 (0.3); 3.6879 (16.0); 3.6740 (0.6); 3.6661 (0.5); 3.3218 (2.0); 3.0940 (7.8); 3.0513 (11.6); 2.5013 (8.3); −0.0002 (5.9)
I-02: ¹H-NMR(499.9 MHz, d₆-DMSO):
δ = 8.6499 (1.9); 8.6382 (3.3); 8.6265 (1.8); 7.7377 (0.9); 7.7212 (1.0); 7.6925 (11.0); 7.6760 (11.8); 7.3803 (10.5); 7.3639 (9.0);
7.3214 (0.7); 7.3050 (0.6); 6.3006 (1.6); 6.2913 (4.4); 6.2818 (4.5); 6.2722 (1.6); 4.3871 (0.4); 4.3706 (0.5); 4.3398 (9.7);
4.3279 (10.0); 4.3159 (0.9); 3.3355 (12.0); 3.1773 (0.4); 3.1023 (13.2); 3.0787 (16.0); 2.5162 (4.5); 2.5127 (8.5); 2.5091 (11.1);
2.5056 (7.8); 2.5021 (3.6); 1.6442 (1.1); 1.6345 (2.1); 1.6288 (2.3); 1.6240 (1.8); 1.6193 (3.8); 1.6124 (1.7); 1.6097 (2.2);
1.6039 (2.1); 1.5941 (1.0); 0.7273 (1.8); 0.7170 (5.1); 0.7113 (10.3); 0.7076 (8.5); 0.7019 (9.2); 0.6975 (8.5); 0.6935 (10.0);
0.6881 (4.5); 0.6822 (4.9); 0.6779 (8.0); 0.6724 (3.8); 0.6623 (1.1); 0.6573 (0.5)
I-03: ¹H-NMR(300.2 MHz, d₆-DMSO):
δ = 8.6247 (0.6); 7.7644 (1.8); 7.7577 (0.6); 7.7419 (0.6); 7.7351 (2.1); 7.3026 (2.1); 7.2957 (0.6); 7.2800 (0.6); 7.2733 (1.8);
6.3328 (0.7); 6.3169 (0.8); 3.3388 (16.0); 3.0742 (3.4); 2.9418 (3.3); 2.5344 (2.8); 2.5284 (6.0); 2.5223 (8.2); 2.5162 (5.8);
2.5102 (2.6); 0.0320 (0.5); 0.0212 (15.0); 0.0102 (0.5)
I-04: ¹H-NMR(300.2 MHz, CDC13):
δ = 7.7307 (2.9); 7.7031 (3.4); 7.5913 (0.4); 7.4047 (0.4); 7.3656 (2.5); 7.3384 (2.2); 7.2986 (2.8); 5.8988 (0.4); 5.8841 (1.3);
5.8694 (1.4); 5.8547 (0.5); 4.8513 (0.9); 4.8299 (4.0); 3.7659 (2.2); 3.7549 (16.0); 3.2587 (4.5); 3.1761 (1.2); 2.2346 (0.3);
2.2229 (0.4); 2.2088 (0.6); 2.1943 (0.5); 2.1827 (0.4); 1.4645 (0.4); 1.0705 (0.6); 1.0555 (1.5); 1.0457 (2.2); 1.0362 (1.4);
1.0307 (1.8); 1.0199 (0.7); 0.9413 (0.7); 0.9304 (2.0); 0.9208 (1.4); 0.9154 (1.5); 0.9038 (2.2); 0.8940 (1.4); 0.8794 (0.6);
0.0329 (3.0)
I-05: ¹H-NMR(300.2 MHz, d₆-DMSO):
δ = 8.3437 (3.1); 7.9785 (1.8); 7.9751 (1.8); 7.7638 (1.2); 7.7353 (1.8); 7.6289 (1.4); 7.6236 (1.4); 7.6006 (1.0); 7.5954 (0.9);
6.3327 (0.4); 6.3163 (1.1); 6.3003 (1.2); 6.2841 (0.4); 3.9078 (10.5); 3.8820 (0.5); 3.3398 (16.0); 3.1137 (3.7); 3.0595 (7.9);
2.5343 (6.7); 2.5284 (13.9); 2.5223 (19.0); 2.5162 (13.7); 2.5103 (6.4); 0.0321 (0.6); 0.0213 (18.4); 0.0104 (0.8)
I-06: ¹H-NMR(300.2 MHz, CDC13):
δ = 7.6539 (4.3); 7.6265 (5.2); 7.3059 (4.5); 7.2993 (13.0); 7.2795 (3.5); 5.9395 (0.5); 5.9245 (1.5); 5.9086 (2.2); 5.8934 (1.7);
5.8784 (0.5); 5.1048 (0.8); 2.4437 (16.0); 1.6088 (4.3); 1.2917 (1.8); 0.0485 (0.4); 0.0377 (10.2); 0.0268 (0.4)
I-07: ¹H-NMR(300.2 MHz, d₆-DMSO):
δ = 8.3274 (4.6); 8.0283 (3.1); 7.7285 (0.5); 7.7025 (3.9); 7.6999 (4.3); 7.6976 (3.9); 7.6928 (3.0); 7.6645 (0.4); 6.3249 (0.5);
6.3091 (1.8); 6.2931 (1.9); 6.2771 (0.6); 3.9145 (0.3); 3.8972 (15.1); 3.8752 (1.3); 3.8677 (0.4); 3.3422 (16.0); 3.1140 (5.8);
3.0607 (12.0); 2.5344 (5.2); 2.5283 (11.3); 2.5222 (15.6); 2.5161 (11.3); 2.5101 (5.2); 0.0319 (0.7); 0.0211 (23.8); 0.0101 (0.8)
I-08: ¹H-NMR(300.2 MHz, CDC13):
δ = 7.7503 (2.0); 7.7230 (2.4); 7.4418 (1.5); 7.4160 (1.4); 7.2982 (35.7); 5.9441 (0.5); 5.9293 (1.5); 5.9146 (1.6); 5.8998 (0.6);
4.8788 (0.3); 4.8485 (5.0); 3.6935 (16.0); 3.3304 (3.6); 3.2681 (0.6); 2.2307 (11.6); 0.0479 (1.6); 0.0372 (46.7); 0.0294 (1.5);
0.0279 (1.4); 0.0263 (1.7)
I-09: ¹H-NMR(300.2 MHz, d₆-DMSO):
δ = 8.3394 (1.2); 8.2794 (2.8); 8.1924 (1.2); 8.0253 (0.6); 7.9504 (1.5); 7.9470 (1.5); 7.6581 (0.9); 7.6300 (1.6); 7.5769 (0.6);
7.5718 (0.6); 7.5606 (0.9); 7.5539 (1.2); 7.5486 (1.1); 7.5258 (0.7); 7.5205 (0.7); 6.2746 (1.0); 6.2586 (1.0); 6.2424 (0.4);
5.7774 (0.6); 3.1130 (7.5); 3.0980 (16.0); 2.5341 (2.9); 2.5282 (6.5); 2.5221 (9.1); 2.5160 (6.8); 2.5101 (3.4); 0.0208 (10.2);
0.0099 (0.5)
I-10: ¹H-NMR(300.2 MHz, d₆-DMSO):
δ = 8.1194 (1.0); 8.0912 (1.3); 7.9101 (1.4); 7.8818 (1.1); 7.6354 (0.4); 7.6147 (0.4); 6.4057 (0.5); 6.3898 (0.5); 3.3489 (16.0);
2.5345 (0.8); 2.5286 (1.8); 2.5225 (2.6); 2.5165 (1.9); 2.5106 (0.9); 0.0203 (3.1)
I-11: ¹H-NMR(300.2 MHz, d₆-DMSO):
δ = 8.8103 (1.1); 8.7254 (0.8); 8.7108 (0.8); 7.9498 (1.5); 7.9214 (2.2); 7.8030 (2.3); 7.7747 (1.5); 6.3756 (0.5); 6.3602 (0.7);
6.3449 (0.5); 3.3481 (16.0); 2.5345 (1.0); 2.5285 (2.1); 2.5225 (3.0); 2.5164 (2.2); 2.5105 (1.0); 1.3904 (5.8); 0.7864 (0.4);
0.7627 (1.3); 0.7494 (0.6); 0.6563 (0.7); 0.6423 (1.4); 0.6362 (1.2); 0.6179 (0.4); 0.0200 (4.5)
I-12: ¹H-NMR(300.2 MHz, d₆-DMSO):
δ = 8.7171 (1.0); 8.6156 (1.2); 8.6006 (1.2); 7.9740 (4.2); 7.9516 (1.9); 7.9456 (6.5); 7.8317 (6.5); 7.8033 (4.3); 6.3923 (0.7);
6.3766 (2.3); 6.3608 (2.5); 6.3448 (0.8); 3.3498 (16.0); 2.8251 (9.0); 2.8099 (9.0); 2.5345 (2.0); 2.5285 (4.3); 2.5224 (6.1);
2.5164 (4.4); 2.5106 (2.1); 0.0197 (8.3)
I-13: ¹H-NMR(300.2 MHz, d₆-DMSO):
δ = 8.7563 (1.0); 8.7289 (3.5); 8.0017 (5.0); 7.9734 (7.2); 7.8393 (7.2); 7.8110 (5.1); 6.3925 (0.6); 6.3778 (1.6); 6.3622 (1.7);
6.3463 (0.6); 3.3523 (16.0); 3.1958 (2.9); 3.1748 (4.4); 3.1543 (3.0); 2.5344 (1.1); 2.5285 (2.4); 2.5225 (3.3); 2.5165 (2.4);
2.5106 (1.2); 1.0852 (0.6); 1.0817 (0.7); 1.0749 (0.7); 1.0699 (0.6); 1.0589 (1.2); 1.0482 (0.6); 1.0428 (0.7); 1.0362 (0.7);
1.0325 (0.7); 1.0164 (0.4); 0.4917 (1.0); 0.4775 (3.0); 0.4714 (3.4); 0.4655 (1.6); 0.4581 (1.7); 0.4504 (3.2); 0.4443 (3.0);
0.4315 (1.3); 0.2799 (1.3); 0.2659 (3.6); 0.2618 (3.7); 0.2506 (3.2); 0.2456 (3.7); 0.2306 (0.9); 0.0187 (4.4)
I-14: ¹H-NMR(300.2 MHz, d₆-DMSO):
δ = 8.7213 (0.4); 8.7067 (0.4); 7.8976 (0.7); 7.8755 (0.3); 7.8691 (1.2); 7.7885 (1.2); 7.7598 (0.7); 6.3690 (0.4); 3.3474 (16.0);
3.1851 (1.6); 3.1698 (1.6); 2.5346 (1.0); 2.5286 (2.1); 2.5226 (2.9); 2.5165 (2.1); 2.5108 (1.0); 0.0207 (3.1)
I-15: ¹H-NMR(300.2 MHz, d₆-DMSO):
δ = 10.5592 (0.4); 8.7293 (0.8); 8.7148 (0.8); 7.8638 (1.2); 7.8418 (0.7); 7.8352 (3.0); 7.7898 (2.9); 7.7673 (0.5); 7.7611 (1.3);
6.3846 (0.5); 6.3692 (0.7); 6.3539 (0.5); 3.6131 (1.0); 3.5947 (1.2); 3.5901 (1.2); 3.5718 (1.0); 3.3479 (16.0); 2.5345 (0.9);
2.5286 (2.1); 2.5225 (2.9); 2.5165 (2.1); 2.5106 (1.0); 1.2635 (0.4); 0.5402 (0.9); 0.5341 (1.1); 0.5207 (0.6); 0.5132 (1.0);
0.5070 (1.0); 0.4941 (0.4); 0.3621 (0.4); 0.3479 (1.2); 0.3443 (1.2); 0.3328 (1.0); 0.3277 (1.2); 0.0207 (3.5)
I-16: ¹H-NMR(300.2 MHz, CDC13):
δ = 7.7103 (0.5); 7.7018 (3.3); 7.6952 (1.2); 7.6794 (1.3); 7.6727 (3.8); 7.6642 (0.6); 7.3590 (4.1); 7.3523 (1.3); 7.3361 (1.2);
7.3297 (3.4); 7.2986 (11.3); 6.6422 (0.6); 6.6260 (1.7); 6.6097 (1.8); 6.5935 (0.6); 3.1630 (10.0); 3.0735 (10.0); 1.9781 (16.0);
1.5897 (8.9); 0.0476 (0.6); 0.0369 (13.1); 0.0260 (0.6)
I-17: ¹H-NMR(400.1 MHz, d₆-DMSO):
δ = 8.6262 (4.5); 7.7508 (11.3); 7.7293 (13.1); 7.4989 (7.1); 7.4794 (16.0); 7.4597 (12.3); 7.4413 (14.8); 7.4209 (7.0); 7.3188
(9.6); 7.3009 (11.6); 7.2837 (4.6); 6.3348 (2.0); 6.3234 (6.2); 6.3117 (6.9); 6.2997 (2.8); 4.0459 (0.5); 4.0281 (0.5); 3.3577
(12.8); 3.3428 (53.5); 3.2238 (0.8); 3.0836 (0.3); 2.5146 (16.7); 2.5104 (23.3); 2.5061 (18.2); 1.9977 (2.0); 1.2004 (0.6); 1.1825
(1.1); 1.1648 (0.5)
I-18: ¹H-NMR(300.2 MHz, CDC13):
δ = 7.7264 (2.7); 7.6976 (3.1); 7.2996 (12.2); 7.2296 (1.6); 7.2072 (1.4); 5.8970 (0.8); 5.8813 (1.3); 5.8661 (1.0); 5.4011 (0.9);
5.3852 (0.8); 3.5351 (0.8); 3.5114 (0.9); 3.4900 (0.6); 3.4680 (0.8); 3.4439 (0.8); 3.1212 (3.2); 3.0418 (3.4); 1.5869 (16.0);
1.3186 (0.9); 1.2947 (1.9); 1.2691 (1.6); 1.2441 (1.7); 1.2204 (0.8); 0.0487 (0.6); 0.0392 (14.8)
I-19: ¹H-NMR(300.2 MHz, CDC13):
δ = 8.0966 (2.6); 7.8913 (2.0); 7.8318 (1.4); 7.8036 (1.6); 7.5672 (1.2); 7.5630 (1.4); 7.5391 (0.9); 7.5349 (1.1); 7.2997 (17.2);
5.9882 (0.6); 5.9723 (0.9); 5.9569 (0.6); 5.3393 (6.7); 5.2171 (0.5); 4.6403 (1.5); 4.6223 (2.8); 4.6049 (1.7); 3.9036 (1.8);
3.8858 (3.3); 3.8681 (1.6); 3.5400 (1.3); 3.5217 (1.3); 3.3308 (12.0); 2.0841 (0.8); 1.5971 (16.0); 1.2980 (0.5); 0.1081 (0.4);
0.0496 (0.8); 0.0387 (22.2)
I-20: ¹H-NMR(300.2 MHz, CDC13):
δ = 7.5698 (0.6); 7.5452 (0.7); 7.4735 (1.7); 7.2995 (1.7); 7.2487 (1.6); 7.2221 (1.4); 5.9643 (0.6); 5.9494 (1.3); 5.9304 (1.4);
5.9154 (0.7); 4.6397 (5.4); 3.7764 (16.0); 3.7405 (1.1); 3.7210 (2.0); 3.7022 (1.2); 2.9183 (1.5); 2.8987 (2.6); 2.8791 (1.4);
2.0763 (1.0); 1.7330 (3.4); 1.3153 (0.4); 1.2916 (0.9); 0.9151 (0.4); 0.0335 (1.9)
I-21: ¹H-NMR(300.2 MHz, d₆-DMSO):
δ = 8.3015 (0.4); 7.3178 (0.3); 7.2765 (0.3); 5.6860 (0.6); 3.3419 (16.0); 2.5343 (1.5); 2.5286 (3.0); 2.5226 (4.1); 2.5166 (3.1);
0.0213 (5.0)
I-22: ¹H-NMR(300.2 MHz, CDC13):
δ = 8.2915 (2.0); 8.2682 (2.0); 8.0393 (3.4); 7.4909 (1.7); 7.4479 (2.0); 7.4346 (1.7); 7.4161 (3.7); 7.4107 (4.2); 7.4016 (1.2);
7.3538 (2.3); 7.3442 (1.8); 7.3284 (1.5); 7.3224 (1.2); 7.2993 (18.9); 5.9809 (1.0); 5.9671 (1.3); 5.9551 (1.0); 5.9408 (0.3);
5.6274 (8.2); 2.0841 (0.9); 1.5866 (16.0); 1.2983 (0.6); 0.0484 (1.0); 0.0387 (25.1)
I-23: ¹H-NMR(300.2 MHz, d₆-DMSO):
δ = 8.6202 (0.4); 8.6050 (0.4); 8.2922 (0.9); 8.1897 (0.7); 7.8739 (0.3); 7.8442 (0.6); 7.7591 (0.4); 7.7543 (0.5); 7.3325 (0.6);
7.3084 (0.8); 7.2978 (0.5); 7.2919 (0.3); 7.2755 (0.3); 7.2516 (0.7); 7.2460 (0.7); 7.2254 (0.4); 6.3170 (0.4); 5.7285 (1.5);
3.3432 (16.0); 2.5287 (3.1); 2.5228 (4.4); 2.5169 (3.4); 0.0213 (5.9)
I-24: ¹H-NMR(300.2 MHz, d₆-DMSO):
δ = 8.7220 (0.8); 8.5538 (0.3); 8.1571 (0.6); 7.6761 (0.5); 7.5864 (0.4); 7.5812 (0.4); 7.3791 (0.3); 7.3647 (1.6); 7.3608 (1.6);
5.6981 (1.4); 3.3451 (16.0); 2.5285 (3.6); 2.5226 (4.9); 2.5168 (3.7); 0.0212 (5.0)
I-25: ¹H-NMR(300.2 MHz, CDC13):
δ = 7.7140 (4.2); 7.6854 (4.9); 7.3002 (13.5); 7.2227 (2.2); 7.1945 (2.1); 5.9033 (0.4); 5.8874 (1.2); 5.8722 (1.9); 5.8571 (1.4);
5.8416 (0.4); 5.4945 (1.2); 4.5286 (0.6); 4.5053 (0.6); 4.4837 (0.4); 4.1718 (0.4); 4.1481 (0.4); 2.9776 (4.6); 2.9151 (2.9);
2.0841 (1.8); 1.5951 (16.0); 1.3222 (0.7); 1.2970 (3.9); 1.2739 (4.1); 1.2501 (5.4); 1.2275 (4.8); 0.0389 (17.5)
I-26: ¹H-NMR(499.9 MHz, d₆-DMSO):
δ = 8.6117 (2.3); 7.8017 (2.0); 7.7364 (5.8); 7.7204 (6.3); 7.4339 (6.2); 7.4180 (5.6); 6.3416 (2.8); 6.3325 (2.8); 6.3234 (1.1);
4.2944 (5.4); 4.2826 (5.4); 3.6123 (16.0); 3.3753 (4.9); 2.5582 (4.5)
I-27: ¹H-NMR(300.2 MHz, CDC13):
δ = 7.7527 (3.0); 7.7252 (4.0); 7.6850 (0.4); 7.5057 (3.5); 7.4784 (2.9); 7.2996 (2.1); 7.1239 (0.7); 7.1076 (6.3); 7.0937 (3.4);
7.0825 (3.7); 7.0726 (0.6); 7.0513 (0.4); 5.8859 (1.0); 5.8697 (1.6); 5.8546 (1.3); 5.8398 (0.4); 5.4682 (1.2); 5.4517 (1.2);
4.8288 (6.9); 4.7106 (0.6); 3.8345 (1.4); 3.7811 (16.0); 3.6601 (1.4); 1.6614 (4.1); 1.3029 (0.7); 1.2945 (0.6); 0.9200 (0.6);
0.0382 (2.4)
I-28: ¹H-NMR(499.9 MHz, d₆-DMSO):
δ = 11.4973 (16.0); 8.6274 (3.9); 8.6188 (3.7); 8.2018 (12.7); 7.7552 (5.0); 7.7420 (4.4); 7.7381 (15.9); 7.7231 (15.1); 7.7193
(3.4); 7.7060 (4.3); 6.3384 (0.8); 6.3296 (2.2); 6.3204 (3.1); 6.3113 (2.1); 6.3019 (0.6); 3.3257 (84.8); 2.5142 (4.2); 2.5106
(8.0); 2.5070 (10.5); 2.5034 (7.4); 2.4998 (3.4); 1.9934 (0.5)
I-29: ¹H-NMR(300.2 MHz, CDC13):
δ = 7.7632 (1.2); 7.7538 (1.2); 7.7342 (1.8); 7.7245 (1.7); 7.5945 (2.3); 7.5668 (1.5); 7.2993 (1.8); 5.9047 (0.7); 5.8900 (1.0);
5.8753 (0.7); 4.7137 (0.4); 4.7028 (0.4); 4.6846 (0.6); 4.6779 (0.6); 4.6488 (0.5); 4.6390 (0.3); 2.8209 (0.3); 2.7922 (0.5);
2.7648 (0.8); 2.6613 (0.4); 2.6492 (0.4); 2.6300 (0.6); 2.6183 (0.7); 2.5747 (0.8); 2.5664 (0.6); 2.5594 (0.5); 2.5278 (0.5);
2.5175 (0.4); 2.4970 (0.4); 2.2445 (0.5); 2.2356 (0.3); 2.2147 (0.7); 2.2040 (0.4); 2.1720 (0.3); 1.6980 (0.5); 1.4362 (16.0);
1.4153 (15.5); 0.0336 (1.8)
I-30: ¹H-NMR(300.2 MHz, d₆-DMSO):
δ = 7.9939 (0.7); 7.9661 (1.0); 7.8348 (1.0); 7.8071 (0.7); 6.3757 (0.4); 6.3602 (0.4); 3.4883 (0.8); 3.4759 (1.3); 3.4646 (0.6);
3.4463 (0.4); 3.3461 (16.0); 3.2910 (4.0); 3.1125 (0.4); 3.0763 (0.5); 2.5283 (4.0); 2.5226 (5.4); 2.5170 (4.1); 0.0208 (5.4)
I-31: ¹H-NMR(300.2 MHz, CDC13):
δ = 7.5729 (4.8); 7.5361 (4.0); 7.2984 (19.7); 5.9178 (1.2); 5.9043 (1.7); 5.8906 (1.2); 5.3306 (1.4); 3.8592 (0.4); 3.8380 (2.4);
3.8295 (2.2); 3.8173 (4.3); 3.8080 (4.2); 3.7956 (2.3); 3.7874 (2.4); 3.7659 (0.4); 2.9951 (1.3); 2.9188 (1.1); 2.8301 (3.0);
2.8079 (6.2); 2.7862 (3.4); 2.6487 (1.1); 2.6095 (2.2); 2.5848 (7.4); 2.5603 (7.6); 2.5358 (2.5); 2.0818 (0.6); 2.0611 (1.3);
2.0395 (4.2); 2.0180 (5.6); 1.9966 (3.8); 1.9746 (1.0); 1.6154 (4.2); 1.2956 (0.4); 1.2512 (7.8); 1.2267 (16.0); 1.2020 (7.2);
0.0465 (0.7); 0.0358 (19.2); 0.0249 (0.7)
I-32: ¹H-NMR(400.1 MHz, d₆-DMSO):
δ = 8.7199 (3.7); 8.7092 (3.6); 8.5018 (0.4); 8.3274 (6.7); 8.1410 (0.5); 8.1192 (0.6); 8.0844 (4.3); 8.0622 (4.6); 8.0094 (3.9);
7.9878 (4.9); 7.8350 (4.4); 7.8132 (3.2); 7.7476 (5.7); 7.4336 (3.1); 7.4294 (3.0); 7.4116 (2.9); 7.4072 (2.8); 6.3677 (2.1);
6.3567 (3.1); 6.3451 (2.2); 3.3110 (20.2); 3.1149 (16.0); 2.9614 (15.9); 2.8987 (0.6); 2.7405 (0.5); 2.5481 (0.5); 2.5090 (14.4)
I-33: ¹H-NMR(300.2 MHz, CDC13):
δ = 7.8602 (1.0); 7.7506 (0.7); 7.7230 (0.8); 7.5040 (0.7); 7.4991 (0.7); 7.4765 (0.6); 7.4715 (0.6); 7.4010 (0.6); 7.3907 (0.9);
7.3822 (0.6); 7.2989 (20.5); 6.6601 (0.5); 6.6530 (0.7); 6.6461 (0.5); 5.9751 (0.6); 5.9699 (0.4); 5.9602 (0.4); 5.9551 (0.6);
5.9402 (0.3); 3.9886 (0.3); 3.9338 (1.1); 1.5891 (16.0); 1.2918 (1.5); 0.0492 (0.8); 0.0384 (21.3); 0.0276 (0.8)
I-34: ¹H-NMR(300.2 MHz, CDC13):
δ = 7.6746 (0.5); 7.6676 (3.1); 7.6611 (1.0); 7.6452 (1.2); 7.6388 (3.6); 7.6317 (0.6); 7.3347 (0.6); 7.3279 (3.7); 7.3215 (1.2);
7.2985 (17.2); 5.9273 (1.0); 5.9122 (1.2); 5.8951 (1.0); 5.0473 (0.5); 5.0317 (0.5); 2.5546 (16.0); 1.5818 (14.0); 0.0488 (0.7);
0.0380 (18.4); 0.0271 (0.7)
I-35: ¹H-NMR(499.9 MHz, d₆-DMSO):
δ = 8.2625 (2.6); 8.2458 (2.8); 8.1777 (0.5); 8.1609 (0.6); 8.1439 (0.3); 8.0146 (4.4); 7.8448 (0.4); 7.8280 (0.4); 7.7833 (2.2);
7.7802 (2.4); 7.7666 (2.2); 7.7634 (2.4); 7.2587 (3.2); 7.2444 (3.5); 7.2025 (0.7); 7.1883 (0.7); 6.6458 (3.0); 6.6314 (3.0);
6.5706 (0.7); 6.5563 (0.6); 6.3779 (1.6); 6.3688 (1.7); 6.3595 (0.7); 3.3274 (4.0); 3.2372 (0.5); 3.1702 (0.8); 3.0954 (16.0);
2.5244 (0.5); 2.5208 (0.8); 2.5066 (22.2); 2.5031 (30.5); 2.4997 (23.8); −0.0002 (0.4)
I-36: ¹H-NMR(300.2 MHz, d₆-DMSO):
δ = 7.7961 (0.7); 7.6576 (0.5); 7.6298 (0.6); 7.5525 (0.4); 7.5436 (0.6); 7.5342 (0.4); 7.4010 (0.5); 7.3960 (0.5); 7.3734 (0.4);
7.3683 (0.4); 6.5325 (0.4); 3.3381 (16.0); 2.5343 (2.6); 2.5283 (5.7); 2.5223 (7.9); 2.5162 (5.7); 2.5103 (2.6); 0.0322 (0.4);
0.0213 (12.9); 0.0103 (0.5)
I-37: ¹H-NMR(300.2 MHz, d₆-DMSO):
δ = 8.7301 (0.5); 8.7167 (0.5); 7.6737 (1.1); 7.6674 (0.4); 7.6515 (0.4); 7.6449 (1.4); 7.3989 (1.4); 7.3925 (0.4); 7.3764 (0.4);
7.3701 (1.1); 6.2896 (0.4); 6.2754 (0.3); 3.3384 (16.0); 2.5408 (6.2); 2.5347 (2.3); 2.5285 (4.2); 2.5223 (5.8); 2.5163 (4.2);
2.5103 (1.9); 0.0213 (9.4); 0.0103 (0.3)
I-38: ¹H-NMR(300.2 MHz, CDC13):
δ = 7.6553 (0.4); 7.6473 (2.7); 7.6408 (1.1); 7.6252 (1.0); 7.6185 (2.9); 7.6104 (0.5); 7.5999 (2.7); 7.2987 (10.2); 7.0544 (0.5);
7.0463 (2.8); 7.0398 (0.9); 7.0239 (0.9); 7.0175 (2.5); 7.0097 (0.3); 5.8975 (0.7); 5.8814 (0.9); 5.8658 (0.7); 5.1212 (0.5);
5.1058 (0.5); 3.1175 (0.8); 3.0937 (16.0); 1.6475 (1.8); 1.2925 (0.7); 0.1071 (1.9); 0.0373 (7.2); 0.0264 (0.3)
I-39: ¹H-NMR(300.2 MHz, CDC13):
δ = 7.9842 (5.6); 7.8344 (3.0); 7.8316 (3.0); 7.7965 (1.8); 7.7953 (1.8); 7.7682 (2.9); 7.6813 (2.2); 7.6762 (2.1); 7.6530 (1.4);
7.6479 (1.4); 7.3810 (0.5); 7.3750 (0.5); 7.3635 (0.7); 7.3560 (1.1); 7.3432 (3.1); 7.3392 (3.0); 7.3282 (4.9); 7.3207 (6.0);
7.3066 (1.1); 7.2986 (12.6); 7.1860 (0.4); 7.1697 (2.2); 7.1584 (2.1); 7.1452 (1.8); 7.1380 (1.5); 5.9323 (0.6); 5.9175 (1.8);
5.9026 (1.9); 5.8876 (0.7); 5.3836 (0.4); 5.3524 (0.4); 5.3119 (7.3); 3.7772 (0.8); 3.2145 (16.0); 3.1892 (0.5); 3.0550 (2.6);
0.1046 (0.4); 0.0320 (4.1)
I-40: ¹H-NMR(300.2 MHz, CDC13):
δ = 7.9280 (1.1); 7.8384 (2.7); 7.8109 (3.5); 7.6393 (3.4); 7.6118 (2.6); 7.2989 (17.7); 5.9720 (0.8); 5.9565 (1.3); 5.9411 (0.9);
5.1516 (0.7); 5.1356 (0.7); 2.1742 (13.2); 2.0840 (0.3); 1.5949 (16.0); 1.2948 (0.7); 0.1076 (1.3); 0.0490 (0.8); 0.0382 (19.1)
I-41: ¹H-NMR(300.2 MHz, CDC13):
δ = 7.7286 (1.6); 7.6999 (1.9); 7.2997 (13.7); 7.2350 (1.1); 7.2101 (1.0); 5.9032 (0.6); 5.8870 (0.9); 5.8718 (0.7); 5.3696 (0.6);
5.3528 (0.6); 4.0454 (0.5); 4.0336 (0.4); 2.9969 (2.2); 2.9378 (1.5); 2.0844 (0.5); 1.8852 (0.9); 1.8800 (0.9); 1.8491 (0.9);
1.7968 (0.5); 1.7424 (0.4); 1.7038 (0.4); 1.5888 (16.0); 1.5284 (0.6); 1.4831 (0.8); 1.4386 (0.9); 1.3964 (0.7); 0.0494 (0.7);
0.0386 (17.9)
I-42: ¹H-NMR(300.2 MHz, CDC13):
δ = 8.1375 (0.4); 8.0538 (0.8); 7.6446 (0.9); 7.6271 (2.6); 7.6003 (3.0); 7.5396 (7.8); 7.4877 (0.4); 7.4117 (0.4); 7.3831 (0.4);
7.3626 (0.6); 7.2988 (129.4); 7.2740 (6.0); 7.2465 (3.4); 7.1846 (0.4); 6.9474 (0.6); 5.9807 (1.8); 5.9672 (3.9); 5.9480 (4.1);
5.9343 (2.0); 5.6372 (1.9); 5.5857 (0.4); 5.3206 (1.3); 5.2740 (0.3); 4.8593 (0.8); 4.8020 (10.3); 4.7397 (0.6); 4.6816 (6.8);
3.9763 (0.9); 3.9311 (0.3); 3.9053 (2.2); 3.8861 (4.2); 3.8667 (2.5); 3.8080 (0.4); 3.7799 (0.7); 3.7545 (2.8); 3.7378 (5.7);
3.7186 (3.1); 3.6861 (0.8); 3.6685 (0.6); 3.6508 (0.5); 3.6309 (0.5); 3.6099 (0.5); 3.5837 (0.5); 3.5689 (0.6); 3.5214 (0.4);
3.4509 (0.3); 3.1996 (0.4); 3.1035 (0.6); 3.0259 (1.9); 3.0115 (1.3); 2.9963 (8.2); 2.9791 (6.0); 2.9599 (3.7); 2.9226 (7.0);
2.9091 (2.1); 2.8570 (0.4); 2.8195 (0.4); 2.6547 (6.2); 2.5108 (2.8); 2.4860 (8.2); 2.4617 (8.6); 2.4369 (3.4); 2.2149 (0.3);
2.1834 (0.5); 2.1584 (0.4); 1.7788 (0.4); 1.6213 (7.5); 1.4542 (0.6); 1.4386 (0.6); 1.3611 (0.5); 1.3120 (0.7); 1.2909 (2.5);
1.2720 (1.3); 1.2533 (8.3); 1.2414 (6.4); 1.2286 (16.0); 1.2173 (10.5); 1.2040 (8.4); 1.1924 (5.3); 1.1659 (1.1); 1.1405 (1.1);
1.1223 (0.9); 1.1090 (0.7); 0.9489 (0.3); 0.9147 (0.5); 0.8896 (0.4); 0.8773 (0.3); 0.2314 (0.5); 0.0479 (4.0); 0.0369 (128.5);
0.0262 (6.1); −0.0289 (0.8); −0.1616 (0.6)
I-43: ¹H-NMR(400.1 MHz, d₆-DMSO):
δ = 8.6049 (3.7); 8.5191 (2.6); 7.6188 (4.4); 7.5984 (4.8); 7.2328 (4.8); 7.2123 (4.3); 6.2838 (0.8); 6.2725 (1.9); 6.2608 (1.9);
6.2495 (0.7); 3.3117 (15.2); 2.8992 (0.9); 2.7405 (0.8); 2.5090 (7.2); 1.8690 (16.0); 1.7769 (0.8); 1.3677 (0.6); 1.2477 (1.1);
1.2258 (4.3); 1.2180 (3.5); 1.1887 (3.5); 1.1810 (4.2); 1.1591 (0.9)
I-44: ¹H-NMR(300.2 MHz, CDC13):
δ = 8.0453 (1.9); 7.6579 (3.3); 7.6450 (0.8); 7.6306 (3.8); 7.5193 (0.4); 7.4915 (0.4); 7.3574 (2.0); 7.3423 (2.3); 7.3195 (4.2);
7.2989 (17.8); 7.2928 (3.8); 7.1137 (1.4); 7.1018 (1.8); 7.0971 (1.5); 7.0849 (1.5); 7.0037 (2.1); 6.9946 (1.7); 6.9034 (0.4);
6.8785 (0.4); 6.4063 (0.8); 5.9347 (1.2); 5.9227 (1.2); 3.9641 (0.9); 3.9523 (1.7); 3.9392 (1.0); 3.9258 (1.2); 3.9132 (2.1);
3.9009 (1.1); 3.7933 (0.9); 3.7489 (2.7); 3.7094 (2.6); 3.6909 (1.0); 3.6648 (2.0); 3.6524 (1.5); 3.6269 (0.9); 3.6141 (0.7);
3.5170 (0.7); 3.4507 (0.3); 3.0228 (1.4); 2.9931 (16.0); 2.9159 (13.3); 2.6471 (3.6); 2.4022 (13.9); 2.1526 (3.9); 1.9311 (0.4);
1.6439 (0.9); 0.0464 (0.8); 0.0356 (21.1); 0.0246 (0.7)
I-46: ¹H-NMR(300.2 MHz, d₆-DMSO):
δ = 8.6787 (0.4); 8.6654 (0.4); 7.8298 (0.6); 7.7990 (0.7); 7.5208 (0.6); 7.5136 (0.8); 7.5066 (0.7); 7.4976 (0.5); 6.2709 (0.3);
3.7559 (0.5); 3.7454 (6.0); 3.7375 (0.9); 3.7265 (0.7); 3.7154 (0.4); 3.7073 (0.4); 3.3462 (16.0); 2.9111 (1.6); 2.7953 (0.5);
2.7878 (0.5); 2.7521 (1.4); 2.7504 (1.2); 2.5342 (1.1); 2.5282 (2.4); 2.5221 (3.3); 2.5160 (2.4); 2.5101 (1.1); 1.9049 (0.5);
1.8845 (0.7); 1.8644 (0.5); 0.0203 (2.5)
I-47: ¹H-NMR(300.2 MHz, d₆-DMSO):
δ = 10.3128 (0.5); 7.8639 (1.0); 7.8344 (1.5); 7.6916 (1.5); 7.6622 (1.1); 7.3821 (1.5); 7.3747 (0.5); 7.3597 (0.5); 7.3522 (1.7);
6.9761 (1.7); 6.9686 (0.5); 6.9536 (0.4); 6.9462 (1.4); 6.2924 (0.4); 6.2765 (0.5); 3.3468 (16.0); 2.9107 (1.1); 2.7520 (0.9);
2.7503 (0.9); 2.5342 (1.4); 2.5282 (2.9); 2.5221 (4.0); 2.5160 (2.9); 2.5101 (1.4); 1.5652 (7.1); 0.0199 (2.9)
I-48: ¹H-NMR(300.2 MHz, d₆-DMSO):
δ = 9.4246 (1.6); 8.5253 (0.5); 7.8123 (2.4); 7.7827 (3.6); 7.6706 (3.6); 7.6643 (1.1); 7.6412 (2.4); 6.2952 (0.4); 6.2793 (1.2);
6.2633 (1.3); 6.2473 (0.4); 3.3478 (16.0); 2.9105 (0.6); 2.7517 (0.6); 2.5339 (1.6); 2.5279 (3.4); 2.5218 (4.6); 2.5158 (3.3);
2.5099 (1.5); 1.4336 (9.6); 1.1479 (0.8); 1.1354 (2.3); 1.1264 (2.5); 1.1146 (0.9); 0.6960 (1.0); 0.6836 (3.0); 0.6744 (2.9);
0.6617 (0.9); 0.0196 (6.1)
I-49: ¹H-NMR(300.2 MHz, d₆-DMSO):
δ = 7.5751 (0.6); 7.5472 (0.9); 7.3953 (0.8); 7.3675 (0.6); 7.2983 (0.5); 7.2943 (0.6); 7.2703 (0.7); 7.2204 (1.0); 7.2130 (0.6);
7.1937 (0.6); 7.1865 (0.4); 6.3069 (0.3); 6.2909 (0.4); 3.4038 (3.5); 3.3460 (16.0); 2.9107 (0.4); 2.7518 (0.4); 2.5339 (1.4);
2.5279 (2.9); 2.5219 (3.9); 2.5158 (2.8); 2.5099 (1.3); 0.0201 (4.0)
I-50: ¹H-NMR(300.2 MHz, d₆-DMSO):
δ = 8.8080 (0.4); 8.1167 (0.5); 8.1110 (2.3); 8.1048 (0.9); 8.0887 (1.0); 8.0822 (3.0); 8.0767 (0.7); 7.9079 (0.6); 7.9025 (3.1);
7.8961 (1.0); 7.8800 (0.9); 7.8736 (2.3); 7.8681 (0.5); 6.4199 (0.4); 6.4042 (1.1); 6.3883 (1.2); 6.3725 (0.4); 3.9256 (0.5);
3.9062 (10.5); 3.3467 (16.0); 2.5341 (1.6); 2.5281 (3.2); 2.5220 (4.3); 2.5158 (3.1); 2.5099 (1.5); 0.0191 (4.0)
I-51: ¹H-NMR(300.2 MHz, d₆-DMSO):
δ = 9.8021 (0.6); 7.8541 (1.8); 7.8260 (2.2); 7.7714 (2.1); 7.7428 (1.0); 7.5267 (0.4); 7.5000 (0.6); 7.4532 (0.5); 7.4278 (1.0);
7.4018 (0.6); 7.3855 (0.6); 7.3806 (0.9); 7.3757 (0.5); 7.3551 (0.4); 6.3595 (0.6); 6.3437 (0.6); 3.7086 (6.7); 3.3476 (16.0);
2.9103 (0.4); 2.5338 (1.5); 2.5279 (3.1); 2.5219 (4.2); 2.5158 (3.0); 2.5099 (1.4); 0.0194 (3.5)
I-52: ¹H-NMR(300.2 MHz, d₆-DMSO):
δ = 8.8448 (0.4); 7.9733 (0.5); 7.8012 (1.1); 7.7730 (1.3); 7.5289 (1.3); 7.5007 (1.1); 7.4468 (0.5); 7.4367 (0.4); 7.4288 (0.3);
7.4158 (0.4); 7.4082 (0.4); 7.3817 (0.3); 7.3562 (0.7); 7.3470 (0.9); 7.3208 (0.3); 6.3607 (0.4); 6.3448 (0.4); 3.5111 (1.8);
3.3469 (16.0); 2.9110 (3.8); 2.7522 (3.2); 2.7506 (3.0); 2.5342 (1.4); 2.5283 (2.8); 2.5222 (3.9); 2.5161 (2.8); 2.5102 (1.3);
0.0207 (4.0)
I-53: ¹H-NMR(300.2 MHz, d₆-DMSO):
δ = 8.5530 (0.4); 7.6380 (1.4); 7.6317 (0.5); 7.6154 (0.5); 7.6092 (1.6); 7.5228 (1.5); 7.5157 (0.5); 7.5006 (0.5); 7.4933 (1.7);
7.1911 (1.6); 7.1848 (0.5); 7.1684 (0.5); 7.1624 (1.4); 7.0982 (1.7); 7.0909 (0.5); 7.0758 (0.5); 7.0686 (1.5); 6.3036 (0.6);
6.2877 (0.6); 3.8300 (6.8); 3.3459 (16.0); 2.5342 (1.7); 2.5282 (3.6); 2.5222 (5.0); 2.5161 (3.6); 2.5103 (1.7); 0.0202 (4.8)
I-54: ¹H-NMR(300.2 MHz, d₆-DMSO):
δ = 8.7073 (0.8); 7.8890 (1.8); 7.8827 (0.8); 7.8668 (1.1); 7.8602 (4.4); 7.8222 (4.4); 7.8157 (1.1); 7.7994 (0.8); 7.7934 (1.7);
7.7006 (0.3); 7.6911 (2.6); 7.6858 (1.0); 7.6626 (2.9); 7.6533 (0.4); 7.1319 (0.4); 7.1222 (3.6); 7.1153 (1.1); 7.0993 (1.0);
7.0925 (3.2); 7.0826 (0.4); 6.3913 (0.3); 6.3757 (1.0); 6.3599 (1.1); 6.3440 (0.4); 3.8044 (14.1); 3.3476 (16.0); 2.5342 (1.7);
2.5282 (3.6); 2.5222 (4.9); 2.5161 (3.5); 2.5102 (1.6); 0.0192 (7.0)
I-55: ¹H-NMR(300.2 MHz, d₆-DMSO):
δ = 9.5609 (1.3); 8.7280 (0.4); 7.9140 (1.1); 7.8857 (2.1); 7.8191 (2.1); 7.7908 (1.1); 6.3752 (0.7); 6.3594 (0.7); 3.3472 (16.0);
2.9108 (0.6); 2.7518 (0.5); 2.6121 (11.2); 2.5341 (1.6); 2.5281 (3.2); 2.5221 (4.4); 2.5161 (3.1); 2.5102 (1.5); 0.0199 (3.1)
I-56: ¹H-NMR(300.2 MHz, CDC13):
δ = 8.2669 (6.2); 8.2606 (2.1); 8.2443 (2.5); 8.2379 (6.9); 8.2327 (1.3); 7.9344 (7.5); 7.9279 (2.3); 7.9116 (2.3); 7.9053 (5.8);
7.8999 (1.0); 7.2984 (22.8); 5.9818 (1.2); 5.1853 (1.1); 2.9317 (2.2); 2.9064 (7.1); 2.8811 (7.4); 2.8559 (2.5); 1.5990 (5.1);
1.4637 (7.8); 1.4384 (16.0); 1.4131 (7.3); 0.0475 (0.9); 0.0367 (25.2); 0.0258 (0.9)
I-57: ¹H-NMR(300.2 MHz, CDC13):
δ = 7.8700 (0.6); 7.8412 (1.0); 7.7670 (1.1); 7.7381 (0.6); 7.2986 (22.6); 4.1844 (0.6); 4.1759 (0.6); 4.1668 (0.6); 4.1587 (0.6);
2.3644 (0.4); 2.3559 (0.9); 2.3474 (0.4); 1.5836 (16.0); 0.0484 (0.8); 0.0376 (23.5); 0.0267 (0.9)
I-58: ¹H-NMR(300.2 MHz, CDC13):
δ = 7.9714 (2.5); 7.9656 (1.0); 7.9490 (1.2); 7.9427 (4.4); 7.9155 (0.4); 7.9057 (3.7); 7.8990 (1.2); 7.8825 (1.2); 7.8757 (4.1);
7.8643 (4.6); 7.8580 (1.4); 7.8355 (2.5); 7.2989 (10.9); 7.0429 (0.4); 7.0330 (3.9); 7.0263 (1.2); 7.0097 (1.1); 7.0029 (3.6);
6.9933 (0.4); 5.9762 (0.7); 5.9608 (1.0); 5.9449 (0.8); 5.4173 (0.8); 5.4004 (0.8); 3.8907 (16.0); 2.9950 (1.1); 2.9135 (0.9);
1.6018 (3.0); 0.0481 (0.4); 0.0372 (11.5); 0.0262 (0.5)
IV-01: ¹H-NMR(300.2 MHz, CDC13):
δ = 8.1395 (2.8); 8.1337 (1.0); 8.1175 (1.0); 8.1116 (3.0); 7.5304 (2.6); 7.5023 (2.4); 7.2987 (5.9); 4.9172 (6.1); 3.7785 (16.0);
2.2550 (0.3); 2.2435 (0.4); 2.2284 (0.6); 2.2131 (0.4); 2.2018 (0.4); 1.5962 (1.4); 1.4701 (0.4); 1.1268 (0.5); 1.1127 (1.6);
1.1030 (2.1); 1.0975 (1.5); 1.0876 (2.0); 1.0767 (0.7); 0.9502 (0.6); 0.9391 (1.9); 0.9292 (1.4); 0.9124 (1.9); 0.9024 (1.3);
0.8887 (0.5); 0.0377 (7.8); 0.0268 (0.3)
IV-02: ¹H-NMR(300.2 MHz, CDC13):
δ = 8.0439 (4.8); 8.0384 (1.7); 8.0164 (5.2); 7.5902 (0.4); 7.5629 (0.5); 7.3653 (4.2); 7.3387 (3.9); 7.3150 (0.4); 7.3134 (0.4);
7.2982 (0.4); 7.2870 (0.3); 2.4719 (16.0); 2.4554 (1.7); 0.0515 (0.4)
IV-03: ¹H-NMR(499.9 MHz, CDC13):
δ = 8.1969 (2.3); 8.0811 (1.4); 8.0783 (1.3); 8.0642 (1.6); 8.0615 (1.5); 7.9981 (1.9); 7.9205 (1.6); 7.9036 (1.3); 7.2653 (2.2);
3.9371 (16.0); 1.7329 (0.6); −0.0002 (2.4)
IV-04: ¹H-NMR(300.2 MHz, CDC13):
δ = 8.1404 (2.8); 8.1345 (1.0); 8.1185 (1.0); 8.1125 (3.1); 7.5272 (2.7); 7.4991 (2.5); 7.2989 (13.4); 4.8948 (6.1); 3.7077 (16.0);
2.2545 (13.3); 1.5849 (6.7); 1.4707 (0.7); 0.0491 (0.7); 0.0383 (17.7); 0.0273 (0.7)
IV-05: ¹H-NMR(300.2 MHz, d₆-DMSO):
δ = 8.1320 (0.5); 8.1232 (4.2); 8.1163 (1.4); 8.1007 (1.4); 8.0937 (4.6); 8.0850 (0.6); 7.4339 (0.6); 7.4250 (4.7); 7.4181 (1.4);
7.4025 (1.3); 7.3956 (4.2); 7.3867 (0.5); 3.3377 (16.0); 3.1118 (0.7); 3.0930 (7.8); 3.0042 (0.6); 2.9589 (7.7); 2.5345 (2.6);
2.5285 (5.4); 2.5224 (7.5); 2.5163 (5.4); 2.5103 (2.5); 0.0320 (0.4); 0.0211 (11.3); 0.0102 (0.4)
IV-06: ¹H-NMR(499.9 MHz, CDC13):
δ = 8.5866 (2.5); 8.1062 (1.5); 8.1043 (1.6); 8.0893 (1.7); 8.0874 (1.7); 7.9734 (2.6); 7.5250 (2.0); 7.5080 (2.0); 7.2650 (2.2);
3.9091 (16.0); 1.7349 (0.5); −0.0002 (2.3)
IV-07: ¹H-NMR(300.2 MHz, CDC13):
δ = 8.1393 (7.8); 8.1115 (8.6); 7.5026 (7.4); 7.4745 (6.8); 7.2987 (45.2); 7.2322 (0.4); 6.0119 (1.0); 4.6019 (8.7); 4.5820 (8.5);
2.0831 (0.6); 1.5841 (16.0); 1.4809 (0.7); 1.4655 (1.5); 1.4542 (1.7); 1.4394 (3.1); 1.4240 (1.9); 1.4130 (1.8); 1.3979 (1.0);
1.1081 (1.6); 1.0939 (5.4); 1.0842 (6.2); 1.0791 (5.0); 1.0693 (5.5); 1.0573 (1.7); 0.8654 (1.8); 0.8533 (5.7); 0.8434 (4.7);
0.8271 (5.6); 0.8170 (4.5); 0.8030 (1.5); 0.0490 (2.2); 0.0382 (59.9); 0.0273 (2.7); −0.0282 (0.6)
IV-08: ¹H-NMR(499.9 MHz, CDC13):
δ = 8.0785 (0.5); 8.0738 (4.0); 8.0701 (1.3); 8.0600 (1.4); 8.0563 (4.3); 8.0517 (0.6); 7.5646 (2.6); 7.5473 (2.5); 7.2624 (3.6);
6.8409 (0.9); 3.8162 (16.0); 1.5938 (13.6); −0.0002 (3.2)
IV-09: ¹H-NMR(300.2 MHz, d₆-DMSO):
δ = 12.8685 (0.4); 8.4604 (0.4); 8.4465 (16.0); 8.3062 (6.0); 8.3029 (6.2); 7.9548 (3.6); 7.9495 (3.5); 7.9267 (5.7); 7.9214 (5.6);
7.8361 (6.5); 7.8081 (4.1); 3.3580 (1.4); 2.5343 (2.5); 2.5284 (5.3); 2.5223 (7.3); 2.5163 (5.3); 2.5103 (2.5); 2.0082 (0.4);
0.0170 (7.8)
IV-11: ¹H-NMR(400.1 MHz, d₆-DMSO):
δ = 9.7911 (2.2); 8.1851 (4.0); 8.1642 (4.6); 7.8952 (2.4); 7.8754 (4.6); 7.8545 (4.0); 7.5294 (1.1); 7.5100 (1.7); 7.4561 (1.1);
7.4370 (2.5); 7.4177 (1.8); 7.4056 (2.4); 7.3864 (0.9); 3.7039 (16.0); 3.3104 (5.3); 2.8979 (1.2); 2.7395 (1.2); 2.5126 (3.5);
2.5086 (4.6); 2.5047 (3.5)
IV-12: ¹H-NMR(400.1 MHz, d₆-DMSO):
δ = 8.8891 (11.5); 8.1285 (14.6); 8.1078 (16.0); 7.6353 (15.8); 7.6146 (14.6); 7.4559 (1.8); 7.4372 (10.5); 7.4212 (5.5); 7.4177
(6.1); 7.3992 (5.7); 7.3837 (8.6); 7.3548 (4.7); 7.3501 (3.9); 7.3350 (4.8); 7.3194 (1.6); 7.3152 (1.7); 3.4822 (19.3); 3.3097
(19.6); 2.8985 (0.5); 2.7400 (0.4); 2.5125 (11.3); 2.5086 (14.7); 2.5047(11.1)
IV-13: ¹H-NMR(300.2 MHz, d₆-DMSO):
δ = 8.7918 (1.2); 8.2698 (1.0); 8.2401 (1.0); 8.1484 (2.9); 8.1449 (2.8); 7.8310 (1.0); 7.8232 (1.0); 7.4954 (0.7); 7.4877 (0.7);
7.4658 (0.7); 7.4580 (0.7); 3.3387 (16.0); 3.1368 (3.4); 2.9805 (3.4); 2.5342 (3.0); 2.5283 (6.0); 2.5222 (8.1); 2.5161 (5.8);
2.5102 (2.7); 0.0317 (0.5); 0.0210 (10.8); 0.0100 (0.4)
IV-14: ¹H-NMR(499.9 MHz, CDC13):
δ = 8.3035 (2.3); 8.3021 (2.3); 8.0879 (2.9); 8.0866 (2.9); 7.8960 (1.0); 7.8936 (0.9); 7.8791 (2.1); 7.8767 (2.1); 7.8556 (2.4);
7.8551 (2.4); 7.8388 (1.0); 7.8383 (1.0); 7.2609 (2.6); 4.6493 (2.3); 4.6384 (4.3); 4.6275 (2.2); 3.8849 (2.5); 3.8741 (4.3);
3.8632 (2.2); 3.3065 (16.0); 1.5892 (3.0); 0.0719 (0.5); −0.0002 (2.6)
IV-15: ¹H-NMR(499.9 MHz, d₆-DMSO):
δ = 8.7477 (8.2); 8.5930 (5.6); 7.8608 (2.3); 7.8576 (2.3); 7.8427 (4.9); 7.8395 (5.2); 7.8142 (5.7); 7.7961 (2.5); 7.3940 (1.4);
7.3838 (16.0); 7.3777 (10.0); 7.3735 (10.0); 7.3617 (1.2); 7.3583 (1.4); 7.3566 (1.5); 7.3519 (0.7); 7.3425 (1.6); 7.3368 (1.9);
7.3326 (1.6); 7.3295 (1.5); 7.3251 (2.0); 7.3189 (1.0); 7.3149 (0.8); 7.3135 (0.7); 7.3081 (0.4); 5.7167 (15.8); 3.3477 (5.7);
3.3441 (5.7); 2.5162 (0.5); 2.5127 (1.1); 2.5091 (1.6); 2.5055 (1.2); 2.5020 (0.6)
IV-16: ¹H-NMR(400.1 MHz, d₆-DMSO):
δ = 7.8768 (6.5); 7.8405 (12.2); 3.7642 (4.7); 3.7488 (7.3); 3.7332 (4.9); 3.3075 (9.0); 2.8531 (3.9); 2.8368 (7.7); 2.8206 (4.2);
2.6057 (2.2); 2.5875 (7.3); 2.5692 (7.5); 2.5510 (2.5); 2.5126 (5.9); 2.5086 (7.7); 2.5046 (5.8); 1.9519 (1.2); 1.9357 (4.0);
1.9200 (5.5); 1.9044 (3.8); 1.8883 (1.1); 1.0814 (7.7); 1.0632 (16.0); 1.0449 (7.5)
IV-17: ¹H-NMR(400.1 MHz, d₆-DMSO):
δ = 8.5803 (6.6); 8.3355 (9.1); 8.0484 (2.6); 8.0449 (2.7); 8.0264 (4.6); 8.0228 (4.7); 7.9647 (5.6); 7.9425 (3.5); 7.3418 (1.7);
7.3234 (4.7); 7.3062 (7.1); 7.2825 (3.8); 7.2705 (11.0); 7.2513 (5.9); 5.7404 (16.0); 3.3083 (19.8); 2.5067 (4.1); 2.5025 (5.7);
2.4983 (4.5); −0.0002 (2.1)
IV-18: ¹H-NMR(300.2 MHz, d₆-DMSO):
δ = 7.9408 (6.0); 7.9253 (6.2); 7.9187 (2.1); 7.9029 (2.1); 7.8963 (6.5); 7.8883 (0.9); 7.1536 (0.9); 7.1455 (6.5); 7.1389 (2.1);
7.1231 (2.0); 7.1165 (6.0); 7.1085 (0.8); 3.3397 (16.0); 3.0798 (8.3); 2.9813 (8.3); 2.5338 (2.4); 2.5278 (5.1); 2.5217 (7.0);
2.5156 (5.0); 2.5096 (2.4); 0.0308 (0.4); 0.0199 (10.2); 0.0089 (0.4)
IV-19: ¹H-NMR(499.9 MHz, d₆-DMSO):
δ = 11.5342 (5.5); 8.1444 (15.7); 7.7638 (9.4); 7.7472 (16.0); 7.7057 (13.1); 7.7028 (12.7); 7.6892 (7.5); 7.6862 (7.4); 7.6136
(8.6); 7.6083 (13.5); 7.6028 (8.4); 6.5783 (10.1); 3.3737 (0.4); 3.3232 (138.3); 2.5128 (7.5); 2.5093 (14.9); 2.5057 (20.0);
2.5021 (14.4); 2.4987 (6.9)
IV-20: ¹H-NMR(499.9 MHz, d₆-DMSO):
δ = 8.6331 (15.6); 8.2472 (7.0); 8.2454 (7.2); 7.9301 (2.7); 7.9270 (2.5); 7.9131 (6.8); 7.9100 (6.8); 7.8922 (9.2); 7.8753 (3.5);
7.3706 (2.8); 7.3675 (1.2); 7.3557 (6.9); 7.3453 (2.4); 7.3413 (7.5); 7.3318 (0.3); 7.3107 (10.0); 7.3057 (2.6); 7.3027 (3.6);
7.2968 (5.9); 7.2908 (2.2); 7.2865 (3.9); 7.2818 (0.9); 7.2750 (0.9); 7.2722 (1.3); 5.6720 (16.0); 3.3267 (39.8); 2.5259 (0.4);
2.5222 (0.6); 2.5181 (0.8); 2.5116 (9.8); 2.5080 (20.5); 2.5044 (27.9); 2.5007 (19.8); 2.4972 (9.0); 2.0763 (1.2); −0.0002 (0.6)
IV-21: ¹H-NMR(499.9 MHz, d₆-DMSO):
δ = 8.6158 (5.4); 8.6024 (5.5); 8.3561 (9.3); 8.3544 (9.0); 8.0160 (3.8); 7.9932 (3.9); 7.3568 (1.4); 7.3544 (2.2); 7.3513 (0.9);
7.3421 (4.0); 7.3399 (5.8); 7.3375 (4.3); 7.3304 (2.1); 7.3258 (7.8); 7.3163 (0.7); 7.3075 (10.0); 7.3011 (4.0); 7.2939 (4.7);
7.2905 (3.1); 7.2846 (3.4); 7.2788 (0.7); 7.2741 (0.6); 7.2708 (1.0); 7.2676 (0.5); 5.7002 (16.0); 3.3273 (19.5); 2.5171 (1.7);
2.5135 (3.6); 2.5099 (5.0); 2.5062 (3.6); 2.5026 (1.6)
IV-22: ¹H-NMR(499.9 MHz, d₆-DMSO):
δ = 8.8112 (6.0); 8.8098 (6.0); 8.6761 (3.7); 8.6620 (3.8); 8.3321 (0.4); 8.3303 (0.4); 7.6603 (2.8); 7.6360 (2.8); 7.3955 (0.4);
7.3869 (12.2); 7.3780 (16.0); 7.3710 (0.8); 7.3675 (0.5); 7.3631 (0.5); 7.3603 (0.6); 7.3579 (0.6); 7.3501 (1.5); 7.3422 (2.0);
7.3393 (0.9); 7.3333 (1.8); 7.3299 (0.8); 7.3254 (0.9); 7.3223 (0.6); 7.3157 (0.6); 7.2810 (0.4); 5.7066 (12.9); 5.6827 (0.7);
3.3290 (25.9); 2.5172 (1.3); 2.5136 (2.7); 2.5099 (3.8); 2.5062 (2.7); 2.5026 (1.2)
IV-23: ¹H-NMR(300.2 MHz, CDC13):
δ = 8.1760 (7.6); 8.1471 (8.0); 7.3493 (4.5); 7.3228 (4.6); 7.2989 (24.9); 4.1148 (0.6); 4.1039 (1.1); 4.0916 (0.9); 4.0763 (1.1);
4.0650 (2.0); 4.0536 (1.4); 4.0395 (0.8); 4.0258 (1.0); 3.0096 (8.7); 2.9524 (5.6); 1.8955 (3.9); 1.8549 (4.2); 1.8131 (2.2);
1.7491 (1.5); 1.7083 (1.6); 1.5848 (16.0); 1.5358 (2.2); 1.4876 (3.3); 1.4457 (3.8); 1.4030 (2.7); 1.3703 (0.8); 1.3594 (0.9);
1.2166 (0.6); 1.1748 (1.2); 1.1387 (1.0); 0.0486 (1.6); 0.0379 (32.4); 0.0272 (1.8)
IV-24: ¹H-NMR(400.2 MHz, d₆-DMSO):
δ = 7.9528 (1.4); 7.9399 (0.4); 7.9297 (2.0); 7.8361 (1.8); 7.8278 (2.1); 7.8230 (2.2); 3.7637 (2.0); 3.7486 (16.0); 3.7335 (2.1);
3.3261 (50.2); 2.8619 (1.4); 2.8459 (2.7); 2.8299 (1.5); 2.5074 (36.7); 2.5034 (45.9); 1.9256 (0.5); 1.9097 (1.5); 1.8943 (1.9);
1.8792 (1.4); 1.8633 (0.4)
IV-25: ¹H-NMR(300.2 MHz, CDC13):
δ = 7.9581 (0.9); 7.9316 (1.2); 7.9094 (1.2); 7.3404 (1.2); 7.3140 (1.1); 7.2987 (3.4); 4.7419 (2.6); 3.8008 (16.0);
3.7819 (1.2); 2.9836 (1.0); 2.9643 (1.7); 2.9453 (0.9); 1.6212 (0.7); 1.4689 (0.6); 0.1076 (2.6); 0.0366 (4.0)
IV-26: ¹H-NMR(400.1 MHz, d₆-DMSO):
δ = 12.0508 (2.2); 11.8731 (0.4); 8.1631 (1.2); 8.0151 (0.7); 8.0113 (0.7); 7.9953 (0.8); 7.9440 (4.0); 7.8986
(5.2); 7.8850 (2.8); 7.8685 (4.1); 7.8531 (3.3); 7.4808 (1.0); 7.4604 (1.0); 7.4389 (5.3); 7.4192 (4.8); 6.8929 (0.3);
6.6269 (1.4); 4.7832 (8.0); 4.7248 (10.2); 3.7274 (6.6); 3.7127 (13.4); 3.6979 (7.4); 3.3094 (21.3); 3.1954 (0.3);
3.1837 (0.4); 2.9771 (3.1); 2.9632 (5.3); 2.9488 (3.2); 2.8782 (2.7); 2.8637 (4.3); 2.8497 (2.5); 2.8134 (0.4); 2.7260 (0.3);
2.7036 (0.5); 2.6851 (0.5); 2.6283 (0.5); 2.6095 (0.6); 2.5876 (0.5); 2.5125 (8.3); 2.5087 (10.7); 2.5047 (8.4); 2.4651 (4.5);
2.4466 (14.2); 2.4281 (14.7); 2.4097 (4.9); 1.0518 (8.3); 1.0453 (8.0); 1.0338 (16.0); 1.0274 (13.1); 1.0157 (8.4)
IV-27: ¹H-NMR(300.2 MHz, d₆-DMSO):
δ = 11.5477 (1.2); 8.1573 (3.0); 8.1550 (3.3); 8.1528 (3.1); 7.7837 (1.7); 7.7560 (3.9); 7.7187 (3.5); 7.7137 (3.4); 7.6910 (1.6);
7.6861 (1.5); 7.6348 (2.2); 7.6255 (3.2); 7.6163 (2.3); 6.6031 (1.3); 6.6002 (1.6); 6.5968 (1.8); 6.5935 (2.5); 6.5902 (1.7);
6.5868 (1.6); 6.5839 (1.3); 3.3427 (16.0); 2.5339 (1.8); 2.5279 (3.8); 2.5218 (5.2); 2.5156 (3.7); 2.5097 (1.8); 0.0191 (6.3)
IV-28: ¹H-NMR(300.2 MHz, d₆-DMSO):
δ = 8.0120 (0.6); 8.0048 (3.9); 7.9984 (1.4); 7.9824 (1.5); 7.9759 (4.4); 7.9689 (0.7); 7.5117 (0.7); 7.5047 (4.4); 7.4984 (1.5);
7.4823 (1.4); 7.4759 (3.9); 7.4689 (0.6); 3.3390 (16.0); 2.5796 (19.0); 2.5560 (1.0); 2.5342 (2.1); 2.5284 (4.4); 2.5222 (5.8);
2.5161 (4.3); 2.5101 (2.1); 2.5049 (1.1); 1.3761 (0.3); 0.0203 (8.2); 0.0093 (0.3)
IV-29: ¹H-NMR(300.2 MHz, CDC13):
δ = 8.8217 (0.9); 8.1319 (2.1); 8.1256 (0.7); 8.1090 (0.8); 8.1027 (2.5); 8.0951 (0.4); 7.8306 (0.4); 7.8231 (2.6); 7.8166 (0.8);
7.8000 (0.7); 7.7937 (2.1); 7.3180 (2.4); 7.3106 (0.8); 7.2957 (0.9); 7.2882 (2.8); 6.9825 (2.9); 6.9750 (0.9); 6.9601 (0.8);
6.9527 (2.4); 1.6110 (16.0)
IV-30: ¹H-NMR(400.2 MHz, d₆-DMSO):
δ = 8.1219 (4.7); 8.1014 (5.2); 7.9886 (0.7); 7.9693 (0.7); 7.9539 (0.7); 7.5862 (5.0); 7.5657 (4.8); 7.5129 (2.6); 7.5005
(2.8); 7.4743 (0.4); 7.1775 (0.7); 7.1511 (2.7); 7.1437 (3.0); 7.0897 (2.1); 7.0774 (2.5); 7.0683 (1.8); 7.0466 (0.5); 6.8303
(0.5); 3.8080 (2.2); 3.7982 (1.4); 3.7880 (1.6); 3.7789 (2.6); 3.6666 (6.9); 3.6009 (0.5); 3.5744 (0.5); 3.5292 (1.1); 3.4639
(1.5); 3.4391 (2.6); 3.4145 (1.4); 3.3249 (24.9); 3.2466 (0.3); 3.2179 (0.6); 2.9041 (2.0); 2.8936 (5.1); 2.7350 (3.9); 2.5265
(0.9); 2.5217 (1.6); 2.5131 (18.0); 2.5087 (35.7); 2.5041 (47.0); 2.4995 (34.8); 2.4951 (17.3); 2.3861 (16.0); 2.3309 (0.4);
2.0916 (1.5); 2.0570 (2.8); 2.0075 (1.5); 1.9980 (1.6); 1.9822 (1.6); 1.9728 (2.1); 1.9477 (0.9); 1.9379 (0.8); 1.8892 (0.5);
1.8558 (0.6); 1.6599 (0.4); 1.2393 (0.8); −0.0002 (2.6)
IV-31: ¹H-NMR(300.2 MHz, d₆-DMSO):
δ = 8.4573 (0.5); 8.4525 (0.5); 8.4131 (0.4); 8.3853 (0.5); 8.1360 (0.4); 8.1305 (0.3); 6.8253 (0.3); 3.3409 (16.0); 2.5337 (1.3);
2.5277 (2.6); 2.5217 (3.6); 2.5156 (2.6); 2.5097 (1.2); 0.0199 (3.8)
IV-32: ¹H-NMR(300.2 MHz, CDC13):
δ = 8.1629 (4.8); 8.1344 (5.2); 7.4903 (2.0); 7.4628 (5.6); 7.4394 (6.3); 7.4102 (6.8); 7.3858 (3.3); 7.3580 (2.6); 7.3352 (4.4);
7.2994 (31.4); 3.4806 (5.7); 1.5803 (16.0); 0.0498 (1.4); 0.0392 (38.7)
IV-33: ¹H-NMR(300.1 MHz, d₆-DMSO):
δ = 8.1062 (1.4); 8.0998 (0.5); 8.0830 (0.6); 8.0766 (1.8); 7.7819 (1.7); 7.7755 (0.5); 7.7585 (0.5); 7.7522 (1.4); 4.9716 (0.4);
4.9604 (0.3); 4.9424 (0.5); 3.3259 (2.4); 2.6010 (0.5); 2.5952 (0.4); 2.5780 (0.4); 2.5655 (0.6); 2.5522 (0.4); 2.5144 (1.6);
2.5089 (3.2); 2.5028 (4.0); 2.4970 (2.6); 1.3669 (16.0); −0.0002 (0.7)
IV-34: ¹H-NMR(400.1 MHz, CDC13):
δ = 8.2749 (3.2); 8.2705 (1.3); 8.2580 (1.4); 8.2534 (4.0); 8.1000 (4.1); 8.0955 (1.4); 8.0829 (1.3); 8.0785 (3.2); 7.9525 (0.5);
7.9451 (3.7); 7.9401 (1.3); 7.9278 (1.3); 7.9227 (3.9); 7.9154 (0.5); 7.2843 (1.9); 7.0394 (0.5); 7.0321 (3.9); 7.0271 (1.3);
7.0147 (1.3); 7.0097 (3.7); 7.0024 (0.5); 3.8807 (16.0)
IV-35: ¹H-NMR(300.2 MHz, d₆-DMSO):
δ = 9.3543 (0.4); 8.1397 (3.6); 8.1339 (1.3); 8.1176 (1.3); 8.1118 (4.0); 7.6586 (3.5); 7.6307 (3.0); 7.2862 (8.1); 7.2639 (12.4);
4.9481 (5.5); 3.7686 (16.0); 3.3387 (8.3); 2.5343 (2.0); 2.5283 (4.2); 2.5222 (5.8); 2.5161 (4.2); 2.5101 (1.9); 0.0203 (6.0)
IV-36: ¹H-NMR(400.1 MHz, CDC13):
δ = 8.1579 (0.4); 8.1538 (0.7); 8.1496 (3.3); 8.1449 (1.2); 8.1381 (0.3); 8.1327 (1.3); 8.1280 (3.6); 8.1238 (0.7); 7.7126 (0.7);
7.7085 (3.7); 7.7038 (1.3); 7.6915 (1.2); 7.6869 (3.3); 7.6827 (0.6); 7.5508 (0.5); 7.5435 (3.6); 7.5383 (1.2); 7.5264 (1.2);
7.5212(3.8); 7.5140 (0.5); 7.1911 (1.4); 6.9266 (0.5); 6.9194 (3.9); 6.9142 (1.4); 6.9023 (1.2); 6.8971 (3.5); 6.8899 (0.5);
3.7851 (1.1); 3.7575 (16.0)
IV-37: ¹H-NMR(499.9 MHz, d₆-DMSO):
δ = 7.9042 (13.1); 7.8879 (13.9); 7.4883 (12.3); 7.4722 (11.3); 7.2879 (5.7); 7.2721 (14.4); 7.2572 (12.4); 7.2228 (16.0); 7.2078
(9.5); 7.1824 (5.7); 7.1679 (8.5); 7.1535 (3.4); 3.9149 (0.5); 3.5507 (0.4); 3.4132 (65.1); 3.3922 (0.7); 3.3781 (0.4); 3.3373
(20.7); 3.2712 (0.4); 2.8931 (1.1); 2.7354 (1.0); 2.5120 (19.3); 2.5085 (25.1); 2.5051 (18.7); 1.2425 (0.5); 1.0938 (0.4); 0.8591
(0.8); 0.8453 (0.4)
IV-38: ¹H-NMR(300.2 MHz, CDC13):
δ = 8.2472 (2.0); 8.2416 (12.4); 8.2355 (5.0); 8.2194 (4.9); 8.2130 (16.0); 7.9834 (15.2); 7.9772 (5.3); 7.9609 (4.5);
7.9548 (12.4);7.2989 (8.8); 6.3808 (1.9); 3.4056 (8.1); 3.3876 (8.8); 3.3818 (9.3); 3.3637 (8.2); 1.6716 (8.4); 1.1758 (0.5);
1.1670 (0.8); 1.1601 (0.5); 1.1502 (1.6); 1.1411 (1.4); 1.1358 (1.2); 1.1253 (2.7); 1.1150 (1.2); 1.1091 (1.5); 1.0992
(1.9); 1.0908 (0.5); 1.0831 (1.0); 1.0748 (0.7); 1.0587 (0.4); 0.6557 (2.3); 0.6402 (6.3); 0.6358 (7.6); 0.6207 (3.4);
0.6133 (7.2); 0.6093 (6.3); 0.5942 (2.8); 0.5708 (0.4); 0.5452 (0.3); 0.3641 (2.7); 0.3486 (8.6); 0.3292 (8.7);
0.3134 (1.9); 0.0438 (0.4); 0.0329 (11.5); 0.0221 (0.4)
IV-39: ¹H-NMR(400.2 MHz, d₆-DMSO):
δ = 9.5010 (2.8); 8.0157 (0.6); 8.0102 (4.1); 8.0055 (1.6); 7.9931 (1.9); 7.9881 (7.0); 7.9827 (1.2); 7.9276 (1.1); 7.9222 (6.7);
7.9172 (2.0); 7.9049 (1.4); 7.9000 (4.2); 7.8945 (0.6); 3.3285 (12.6); 2.8937 (0.5); 2.7347 (0.4); 2.5273 (0.5); 2.5226 (0.7);
2.5140 (7.3); 2.5095 (14.4); 2.5050 (19.0); 2.5004 (14.0); 2.4959 (6.9); 1.4322 (16.0); 1.1480 (1.4); 1.1385 (4.2); 1.1318 (4.3);
1.1227 (1.6); 0.6896 (1.8); 0.6803 (5.4); 0.6735 (5.4); 0.6636 (1.6)
IV-40: ¹H-NMR(400.2 MHz, d₆-DMSO):
δ = 8.6762 (1.9); 8.6651 (1.9); 8.1661 (7.5); 8.1495 (3.5); 8.1448 (11.6); 8.0610 (11.3); 8.0566 (3.6); 8.0441 (2.9); 8.0397 (7.6);
3.3366 (37.2); 2.8269 (16.0); 2.8155 (15.8); 2.7974 (0.5); 2.7860 (0.3); 2.5567 (0.4); 2.5286 (0.6); 2.5151 (11.0); 2.5108 (21.4);
2.5062 (27.9); 2.5017 (20.9); 2.4973 (10.6); 1.2375 (0.4); −0.0002 (1.3)
IV-41: ¹H-NMR(400.2 MHz, d₆-DMSO):
δ = 10.2419 (3.3); 8.0197 (0.8); 8.0138 (6.3); 8.0090 (2.2); 7.9967 (2.3); 7.9918 (8.4); 7.9861 (1.2); 7.8535 (1.2); 7.8477 (7.9);
7.8428 (2.4); 7.8304 (2.0); 7.8255 (6.3); 7.8198 (0.8); 3.3285 (23.4); 2.5277 (0.6); 2.5229 (0.9); 2.5143 (10.0); 2.5098 (20.1);
2.5052 (26.5); 2.5006 (19.5); 2.4960 (9.6); 2.4081 (2.0); 2.3892 (6.7); 2.3704 (6.8); 2.3516 (2.2); 1.1222 (7.5); 1.1035 (16.0);
1.0846 (7.2)
IV-42: ¹H-NMR(400.3 MHz, CDC13):
δ = 7.8588 (3.6); 7.8544 (1.4); 7.8418 (1.4); 7.8374 (3.8); 7.8326 (0.8); 7.4323 (0.4); 7.4246 (3.6); 7.4195 (1.3); 7.4078
(1.3); 7.4026 (3.9); 7.3951 (0.5); 7.1793 (0.6); 7.1017 (3.8); 7.0972 (1.5); 7.0847 (1.3); 7.0803 (3.7); 7.0757 (0.8); 6.9074
(0.4); 6.8999 (3.9); 6.8947 (1.4); 6.8830 (1.2); 6.8779 (3.6); 6.8703 (0.4); 3.7796 (16.0); 1.5018 (1.6)
IV-43: ¹H-NMR(499.9 MHz, d₆-DMSO):
δ = 8.7699 (2.0); 8.7594 (3.6); 8.7501 (2.0); 8.1692 (10.7); 8.1558 (5.1); 8.1523 (16.0); 8.0792 (15.9); 8.0757 (5.5); 8.0622
(10.8); 3.5020 (1.5); 3.4968 (2.7); 3.4925 (2.1); 3.4863 (11.6); 3.4780 (12.2); 3.4723 (7.8); 3.4637 (9.3); 3.4531 (6.2);
3.4474 (2.3); 3.4423 (1.5); 3.4379 (1.1); 3.4201 (0.4); 3.3197 (37.0); 3.2817 (65.0); 3.1388 (0.3); 2.5253 (0.4); 2.5215
(0.5); 2.5108 (10.3); 2.5073 (21.6); 2.5037 (29.8); 2.5002 (22.2); 2.4968 (11.2); 2.0755 (0.4); −0.0002 (0.8)
IV-44: ¹H-NMR(300.2 MHz, CDC13):
δ = 8.1780 (6.1); 8.1497 (6.6); 7.3562 (2.7); 7.3464 (3.1); 7.3271 (2.9); 7.3185 (2.9); 7.3002 (28.1); 7.2983 (28.5); 3.5722 (0.6);
3.5475 (1.8); 3.5245 (2.0); 3.5065 (1.1); 3.4834 (2.0); 3.4592 (1.9); 3.4360 (0.6); 3.1340 (7.1); 3.0562 (7.5); 1.5816 (15.8);
1.5797 (16.0); 1.3283 (1.9); 1.3049 (3.9); 1.2783 (3.3); 1.2529 (3.8); 1.2289 (1.8); 0.0481 (1.6); 0.0394 (37.3); 0.0374 (37.5)
IV-45: ¹H-NMR(400.0 MHz, d₆-DMSO):
δ = 9.6982 (0.3); 9.6046 (6.9); 8.2429 (14.6); 8.2237 (16.0); 8.0172 (0.3); 7.9967 (0.3); 7.9454 (0.4); 7.9101 (0.5); 7.9005 (0.3);
7.8945 (1.9); 7.8374 (16.0); 7.8181 (15.2); 7.6593 (0.3); 3.9483 (15.7); 3.7782 (0.3); 3.4225 (10.0); 3.3122 (0.7); 3.1690 (8.6);
2.7177 (0.4); 2.6643 (0.7); 2.5381 (10.3); 2.4994 (58.9); 2.3271 (0.4); 1.2561 (0.9); 1.2432 (0.9)
IV-46: ¹H-NMR(400.0 MHz, d₆-DMSO):
δ = 8.3332 (4.0); 8.3140 (14.8); 8.3047 (16.0); 8.2841 (4.6); 7.8960 (0.8); 3.3176 (27.7); 2.8611 (2.3); 2.8426 (6.4); 2.8239
(6.8);2.8055 (2.9); 2.6664 (0.3); 2.5386 (5.5); 2.4990 (35.1); 2.3290 (0.3); 1.3276 (6.3); 1.3088 (12.2); 1.2902 (6.7)
IV-47: ¹H-NMR(400.0 MHz, d₆-DMSO):
δ = 9.6259 (2.3); 8.1534 (2.6); 8.1339 (3.2); 8.0878 (0.3); 8.0707 (0.3); 7.9978 (3.2); 7.9781 (2.7); 3.3204 (8.2); 2.6041 (16.0);
2.5390 (2.0); 2.4995 (10.9)
IV-48: ¹H-NMR(499.9 MHz, d₆-DMSO):
δ = 8.5603 (0.4); 7.9668 (0.4); 7.9503 (0.5); 7.3307 (0.4); 7.3147 (0.4); 3.1570 (16.0); 2.5157 (2.2); 1.8965 (1.5);
1.2756 (0.5); 1.2530 (0.6)
IV-49: ¹H-NMR(300.2 MHz, CDC13):
δ = 8.4859 (0.5); 8.4656 (0.7); 8.4539 (1.2); 8.4342 (1.2); 8.4246 (0.8); 8.4048 (0.7); 8.3344 (4.9); 8.3284 (1.8); 8.3122 (1.9);
8.3059 (6.2); 8.0914 (6.2); 8.0853 (2.0); 8.0689 (1.8); 8.0629 (4.8); 8.0016 (1.2); 7.2987 (14.2); 7.0374 (0.5); 7.0228 (1.4);
7.0127 (1.1); 6.9982 (2.1); 6.9850 (2.0); 6.9731 (1.4); 6.9635 (0.4); 6.9571 (1.2); 6.9480 (0.8); 1.5881 (16.0); 0.0486 (0.6);
0.0378 (17.2); 0.0268 (0.6)
IV-50: ¹H-NMR(300.2 MHz, CDC13):
δ = 8.2284 (4.7); 8.2225 (1.8); 8.2059 (2.2); 8.1999 (5.6); 7.9275 (5.7); 7.8990 (4.5); 7.2985 (9.0); 6.5382 (1.2); 1.6084 (4.4);
1.5459 (16.0); 1.2920 (0.4); 0.9514 (1.0); 0.9282 (4.0); 0.9125 (1.8); 0.8815 (0.6); 0.8621 (0.6); 0.8316 (2.1); 0.8160 (4.2);
0.7921 (1.1); 0.0468 (0.6); 0.0363 (11.8); 0.0254 (0.5)
IV-51: ¹H-NMR(300.2 MHz, d₆-DMSO):
δ = 10.5649 (1.4); 10.5504 (1.4); 8.1535 (8.0); 8.1476 (2.9); 8.1315 (3.4); 8.1252 (11.5); 7.9994 (11.8); 7.9931 (3.5);
7.9770 (2.8); 7.9709 (7.8); 3.8170 (1.1); 3.8101 (0.6); 3.3970 (4.3); 3.2026 (16.0); 3.1873 (16.0); 2.5343 (5.1);
2.5283 (11.1); 2.5223 (15.6); 2.5162 (11.3); 2.5103 (5.4); 0.0311 (0.5); 0.0201 (18.2); 0.0091 (0.7)
IV-52: ¹H-NMR(499.9 MHz, CDC13):
δ = 8.0253 (0.5); 8.0211 (3.4); 8.0174 (1.1); 8.0077 (1.2); 8.0039 (3.5); 7.9998 (0.5); 7.3540 (0.5); 7.3499 (3.5); 7.3462 (1.1);
7.3364 (1.1); 7.3327 (3.3); 7.3286 (0.4); 7.2597 (3.4); 2.5435 (16.0); 2.5121 (0.7); 2.0052 (1.2); 1.5518 (0.4); −0.0002 (3.3)
IV-53: ¹H-NMR(300.2 MHz, d₆-DMSO):
δ = 11.6193 (1.2); 8.2811 (0.9); 8.1326 (0.7); 8.1047 (0.8); 7.8677 (0.8); 7.8397 (0.7); 3.3391 (16.0); 2.5281 (3.4); 2.5221 (4.8);
2.5162 (3.6); 0.0208 (5.0)
IV-54: ¹H-NMR(300.2 MHz, CDC13):
δ = 8.2475 (2.3); 8.2254 (0.8); 8.2191 (2.5); 7.8491 (0.7); 7.7201 (2.5); 7.6917 (2.2); 7.2987 (40.4); 2.2012 (11.7);
1.5879 (16.0); 1.5235 (0.4); 0.1072 (0.7); 0.0488 (1.5); 0.0380 (47.8); 0.0271 (1.7); −0.0287 (0.4)
IV-55: ¹H-NMR(300.2 MHz, d₆-DMSO):
δ = 10.6627 (0.9); 8.1550 (2.6); 8.1491 (1.1); 8.1327 (1.3); 8.1265 (3.5); 7.9639 (3.6); 7.9578 (1.2); 7.9414 (1.1);
7.9353 (2.6); 3.6206 (2.5); 3.5972 (2.6); 3.3471 (16.0); 2.5338 (1.2); 2.5279 (2.3); 2.5219 (3.0); 2.5159 (2.2);
2.5102 (1.0); 1.3020 (0.4); 1.2921 (0.4); 1.2870 (0.3); 1.2761 (0.6); 1.2602 (0.4); 1.2500 (0.4); 0.5644 (0.5);
0.5500 (1.4); 0.5439 (1.6); 0.5303 (0.9); 0.5228 (1.5); 0.5170 (1.4); 0.5038 (0.6); 0.3731 (0.7); 0.3590 (1.8);
0.3559 (1.8); 0.3437 (1.6); 0.3388 (1.7); 0.3235 (0.4); 0.0197 (3.0)
IV-56: ¹H-NMR(300.2 MHz, CDC13):
δ = 8.1850 (2.4); 8.1765 (14.5); 8.1699 (5.9); 8.1538 (5.4); 8.1470 (16.0); 8.1387 (2.7); 7.3506 (6.9);
7.3231 (6.8); 7.2991 (12.5); 4.5771 (1.2); 4.5539 (2.0); 4.5313 (2.2); 4.5091 (1.4); 4.4867 (0.6);
2.9883 (12.6); 2.9288 (7.8); 1.6028 (9.2); 1.3019 (9.1); 1.2797 (11.0); 1.2558 (16.0); 1.2333 (13.9);
1.1503 (0.3); 0.0480 (0.7); 0.0373 (15.3); 0.0265 (1.0)

### BIOLOGICAL DATA

### Example A: In Vivo Preventive Test on Puccinia recondita (Brown Rust on Wheat)

Solvent: 5% by volume of Dimethyl sulfoxide
10% by volume of Acetone
   Emulsifier: 1 μl of Tween® 80 per mg of active ingredient

The active ingredients were made soluble and homogenized in a mixture of Dimethyl sulfoxide/Acetone//Tween80 and then diluted in water to the desired concentration.

The young plants of wheat were treated by spraying the active ingredient prepared as described above. Control plants were treated only with an aqueous solution of Acetone/Dimethyl sulfoxide/Tween®80.

After 24 hours, the plants were contaminated by spraying the leaves with an aqueous suspension of Puccinia recondita spores. The contaminated wheat plants were incubated for 24 hours at 20° C. and at 100% relative humidity and then for 10 days at 20° C. and at 70-80% relative humidity.

The test was evaluated 11 days after the inoculation. 0% means an efficacy which corresponds to that of the control plants while an efficacy of 100% means that no disease was observed.

In this test, the following compound according to the invention showed efficacy between 70% and 79% at a concentration of 500 ppm of active ingredient: I-08.

In this test, the following compounds according to the invention showed efficacy between 80% and 89% at a concentration of 500 ppm of active ingredient: I-17; I-35.

In this test the following compounds according to the invention showed efficacy between 90% and 100% at a concentration of 500 ppm of active ingredient: I-02; I-03; I-04; I-07; I-11; I-12; I-13; I-14; I-15; I-18; I-27; I-34.

In this test, the following compounds according to the invention showed efficacy between 80% and 89% at a concentration of 100 ppm of active ingredient: I-15; I-18; I-30; I-38.

In this test the following compounds according to the invention showed efficacy between 90% and 100% at a concentration of 100 ppm of active ingredient: I-03; I-11; I-12; I-13; I-14; I-16; I-26; I-28; I-34.

### Example B: In Vivo Preventive Test on Uromyces appendiculatus (Bean Rust)

Solvent: 5% by volume of Dimethyl sulfoxide
10% by volume of Acetone
   Emulsifier: 1 μl of Tween® 80 per mg of active ingredient

The active ingredients were made soluble and homogenized in a mixture of Dimethyl sulfoxide/Acetone//Tween80 and then diluted in water to the desired concentration.

The young plants of bean were treated by spraying the active ingredient prepared as described above. Control plants were treated only with an aqueous solution of Acetone/Dimethyl sulfoxide/Tween®80.

After 24 hours, the plants were contaminated by spraying the leaves with an aqueous suspension of Uromyces appendiculatus spores. The contaminated bean plants were incubated for 24 hours at 20° C. and at 100% relative humidity and then for 10 days at 20° C. and at 70-80% relative humidity.

The test was evaluated 11 days after the inoculation. 0% means an efficacy which corresponds to that of the control plants while an efficacy of 100% means that no disease was observed.

In this test, the following compound according to the invention showed efficacy between 80% and 89% at a concentration of 500 ppm of active ingredient: I-21.

In this test the following compounds according to the invention showed efficacy between 90% and 100% at a concentration of 500 ppm of active ingredient: I-01; I-02; I-03; I-04; I-07; 1-08; I-10; I-11; I-12; I-13; I-14; I-15; I-17; I-18; I-19; I-20; I-27; I-34; I-35; I-37.

In this test, the following compound according to the invention showed efficacy between 80% and 89% at a concentration of 100 ppm of active ingredient: I-36.

In this test the following compounds according to the invention showed efficacy between 90% and 100% at a concentration of 100 ppm of active ingredient: I-03; I-10; I-11; I-12; I-13; 1-14; I-15; I-16; I-17; I-18; I-20; I-27; I-34.

### Example C: In Vivo Preventive Test on Phakospora pachyrhizi (Soybean Rust)

Solvent: 5% by volume of Dimethyl sulfoxide
10% by volume of Acetone
   Emulsifier: 1 μl of Tween® 80 per mg of active ingredient

The active ingredients were made soluble and homogenized in a mixture of Dimethyl sulfoxide/Acetone//Tween80 and then diluted in water to the desired concentration.

The young plants of soybean were treated by spraying the active ingredient prepared as described above. Control plants were treated only with an aqueous solution of Acetone/Dimethyl sulfoxide/Tween®80.

After 24 hours, the plants were contaminated by spraying the leaves with an aqueous suspension of Phakospora pachyrhizi spores. The contaminated soybean plants were incubated for 24 hours at 24° C. and at 100% relative humidity and then for 11 days at 24° C. and at 70-80% relative humidity.

The test was evaluated 12 days after the inoculation. 0% means an efficacy which corresponds to that of the control plants while an efficacy of 100% means that no disease was observed.

In this test, the following compound according to the invention showed efficacy between 80% and 89% at a concentration of 500 ppm of active ingredient: I-14.

In this test the following compounds according to the invention showed efficacy between 90% and 100% at a concentration of 500 ppm of active ingredient: I-03; I-10; I-11; I-12; I-13; 1-15; I-17; I-18; I-19; I-20; I-22; I-24; I-27; I-34.

In this test, the following compounds according to the invention showed efficacy between 80% and 89% at a concentration of 100 ppm of active ingredient: I-27; I-38; I-43.

In this test the following compounds according to the invention showed efficacy between 90% and 100% at a concentration of 100 ppm of active ingredient: I-03; I-10; I-11; I-12; I-13; 1-14; I-15; I-16; I-17; I-18; I-22; I-24; I-25; I-26; I-28; I-29; I-30; I-34.

### Example D: In Vivo Preventive Test on Phakopsora Test (Soybeans)

Solvent: 24.5 parts by weight of acetone
24.5 parts by weight of dimethylacetamide
   Emulsifier: 1 part by weight of alkylaryl polyglycol ether

To produce a suitable preparation of active ingredient, 1 part by weight of active ingredient was mixed with the stated amounts of solvent and emulsifier, and the concentrate was diluted with water to the desired concentration.

To test for preventive activity, young plants were sprayed with the preparation of active ingredient at the stated rate of application. After the spray coating had dried on, the plants were inoculated with an aqueous spore suspension of the causal agent of soybean rust (Phakopsora pachyrhiz) and stay for 24 h without light in an incubation cabinet at approximately 24° C. and a relative atmospheric humidity of 95%.

The plants remained in the incubation cabinet at approximately 24° C. and a relative atmospheric humidity of approximately 80% and a day/night interval of 12 h.

The test was evaluated 7 days after the inoculation. 0% means an efficacy which corresponds to that of the untreated control, while an efficacy of 100% means that no disease is observed.

In this test, the following compounds according to the invention showed efficacy between 90% and 100% at a concentration of 250 ppm of active ingredient: I-01; I-02; I-03; I-04; I-08; I-35; I-37

### Example E: Pyricularia oryzae In Vitro Cell Test

Solvent: DMSO
Culture medium: 14.6 g anhydrous D-glucose (VWR), 7.1 g Mycological Peptone (Oxoid), 1.4 g granulated Yeast Extract (Merck), QSP 1 liter
Inoculum: spore suspension

Active ingredients were solubilized in DMSO and the solution used to prepare the required range of concentrations. The final concentration of DMSO used in the assay was ≤1%.

A spore suspension of P. oryzae was prepared and diluted to the desired spore density.

The active ingredients were evaluated for their ability to inhibit spore germination and mycelium growth in liquid culture assay. The active ingredients were added in the desired concentration to the culture medium with spores. After 5 days incubation, fungi-toxicity of compounds was determined by spectrometric measurement of mycelium growth. Inhibition of fungal growth was determined by comparing the absorbance values in wells containing the active ingredients with the absorbance in control wells without active ingredients.

In this test, the following compounds according to the invention showed efficacy between 80% and 89% at a concentration of 20 ppm of active ingredient: I-32

In this test, the following compounds according to the invention showed efficacy between 90% and 100% at a concentration of 20 ppm of active ingredient: I-17; I-27; I-39

### Example F: Colletotrichum lindemuthianum In Vitro Cell Test

Solvent: DMSO
Culture medium: 14.6 g anhydrous D-glucose (VWR), 7.1 g Mycological Peptone (Oxoid), 1.4 g granulated Yeast Extract (Merck), QSP 1 liter
Inoculum: spores suspension

Active ingredients were solubilized in DMSO and the solution used to prepare the required range of concentrations. The final concentration of DMSO used in the assay was:1%.

A spore suspension of C. lindemuthianum was prepared and diluted to the desired spore density.

The active ingredients were evaluated for their ability to inhibit spores germination and mycelium growth in liquid culture assay. The active ingredients were added in the desired concentration to the culture medium with spores. After 6 days incubation, fungi-toxicity of compounds was determined by spectrometric measurement of mycelium growth. Inhibition of fungal growth was determined by comparing the absorbance values in wells containing the active ingredients with the absorbance in control wells without active ingredients.

In this test, the following compounds according to the invention showed efficacy between 70% and 79% at a concentration of 20 ppm of active ingredient: I-06; I-07; I-17; I-33; I-34

In this test, the following compounds according to the invention showed efficacy between 80% and 89% at a concentration of 20 ppm of active ingredient: I-02; I-04; I-14; I-15; I-27

1. A compound of formula (I) or a salt, N-oxide, and/or solvate thereof: 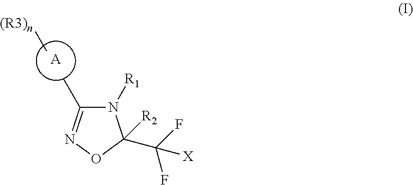
wherein:
A is selected from the group consisting of carbocyclyl, heterocyclyl, aryl and heteroaryl group;
R1 is selected from the group consisting of hydrogen, C₁-C₆-alkyl, C₂-C₆-alkenyl, C₂-C₆-haloalkenyl, C₂-C₆-alkynyl, C₂-C₆-haloalkynyl, C₃-C₈-cycloalkyl, —C(═O)R^{a}, —C(═O)OR^{a} and —C(═O)N(R^{a})₂,
wherein said C₁-C₆-alkyl may be substituted by one or more substituents independently selected from the group consisting of halogen, hydroxy, cyano, mercapto, sulfinyl, sulfonyl, nitro, amino, C₁-C₆-alkoxy, C₁-C₆-haloalkoxy, C₁-C₆-alkylsulfanyl, C₁-C₆-alkylsulfinyl, C₁-C₆-alkylsulfonyl, —C(═O)—C₁-C₆-alkyl, —C(═O)—C₁-C₆-alkoxy, —C═O—C₁-C₆-alkylamino, —C═O—C₁-C₆-dialkylamino, carbocyclyl, heterocyclyl, aryl and heteroaryl,
wherein said carbocyclyl, heterocyclyl, aryl and heteroaryl may be substituted by one or more substituents independently selected from the group consisting of halogen, hydroxy, oxo, cyano, C₁-C₆-alkyl, C₁-C₆-haloalkyl, C₁-C₆-alkoxy and C₁-C₆-haloalkoxy,
wherein said C₃-C₈-cycloalkyl may be substituted by one or more substituents independently selected from the group consisting of halogen, hydroxy, oxo, cyano, C₁-C₆-alkyl, C₁-C₆-haloalkyl, C₁-C₆-alkoxy and C₁-C₆-haloalkoxy,
wherein R^{a} is independently selected from the group consisting of hydrogen, C₁-C₆-alkyl, C₁-C₆-haloalkyl, C₁-C₆-hydroxyalkyl, C₁-C₆-cyanoalkyl, C₂-C₆-alkenyl, C₂-C₆-haloalkenyl, C₂-C₆-hydroxyalkenyl, C₂-C₆-cyanoalkenyl, C₂-C₆-alkynyl, C₂-C₆-haloalkynyl, C₂-C₆-hydroxyalkynyl, C₂-C₆-cyanoalkynyl, carbocyclyl, heterocyclyl, aryl, heteroaryl, —C₁-C₆-alkyl-aryl, —C₁-C₆-alkyl-heterocyclyl, —C₁-C₆-alkyl-heteroaryl and —C₁-C₆-alkyl-C₁-C₆-alkoxy,
wherein any carbocyclyl, heterocyclyl, aryl and heteroaryl group may be substituted by one or more substituents independently selected from the group consisting of halogen, hydroxy, oxo, cyano, C₁-C₆-alkyl, C₁-C₆-haloalkyl, C₁-C₆-alkoxy and C₁-C₆-haloalkoxy;
R2 is selected from the group consisting of hydrogen and C₁-C₆-alkyl,
wherein said C₁-C₆-alkyl may be substituted by one or more substituents independently selected from the group consisting of halogen, hydroxy, cyano, mercapto, sulfinyl, sulfonyl, nitro, amino, C₁-C₆-alkoxy, C₁-C₆-haloalkoxy, C₁-C₆-alkylsulfanyl, C₁-C₆-alkylsulfinyl, C₁-C₆-alkylsulfonyl, —C(═O)—C₁-C₆-alkyl, —C(═O)—C₁-C₆-alkoxy, —C═O—C₁-C₆-alkylamino, —C═O—C₁-C₆-dialkylamino, carbocyclyl, heterocyclyl, aryl and heteroaryl,
wherein said carbocyclyl, heterocyclyl, aryl and heteroaryl may be substituted by one or more substituents independently selected from the group consisting of halogen, hydroxy, oxo, cyano, C₁-C₆-alkyl, C₁-C₆-haloalkyl, C₁-C₆-alkoxy and C₁-C₆-haloalkoxy;
X is F or Cl;
n is 0, 1, 2, 3, 4 or 5;
R3 is independently selected from the group consisting of halogen, hydroxy, oxo, cyano, mercapto, sulfinyl, sulfonyl, nitro, C₁-C₆-alkyl, C₁-C₆-haloalkyl, C₁-C₆-hydroxyalkyl, C₁-C₆-cyanoalkyl, C₁-C₆-aminoalkyl, C₂-C₆-alkenyl, C₂-C₆-haloalkenyl, C₂-C₆-hydroxyalkenyl, C₂-C₆-cyanoalkenyl, C₂-C₆-alkynyl, C₂-C₆-haloalkynyl, C₂-C₆-hydroxyalkynyl, C₂-C₆-cyanoalkynyl, C₁-C₆-alkoxy, C₁-C₆-haloalkoxy, C₁-C₆-alkylsulfanyl, arylsulfanyl, C₁-C₆-alkylsulfinyl, arylsulfinyl, C₁-C₆-alkylsulfonyl, arylsulfonyl, amino, carbocyclyl, heterocyclyl, aryl, heteroaryl, —O-carbocyclyl, —O— heterocyclyl, —O-aryl, —O-heteroaryl, ═N(OR^{b}), —N(OR^{b})C(═O)OR^{b}, —N(OR^{b})C(═O)R^{b}, —SO₂N(R^{b})₂, —OC(═O)R^{b}, —N(R^{b})₂, —NR^{b}C(═O)OR^{b}, —NR^{b}C(═O)N(R^{b})₂, —NR^{b}C(═O)R^{b}, —NR^{b}C(═O)OR^{b}, —NR^{b}C(═S)R^{b}, —NR^{b}C(═S)N(R^{b})₂, —NR^{b}C(═NR^{b})R^{b}, —N═CR^{b}—N(R^{b})₂, —NR^{b}S(═O)₂R^{b}, —OC(═O)N(R^{b})₂, —C(═NR^{b})R^{b}, —C(═NOH)R^{b}, —C(═NOH)N(R^{b})₂, —C(═O)R^{b}, —C(═O)(OR^{b}), —C(═O)N(R^{b})₂, —C(═O)NR^{b}N(R^{b})₂, —C(═O)N(OR^{b})R^{b}, —C(═S)N(R^{b})₂, —C₁-C₆-(halo)alkyl-OR^{b}, —C₁-C₆-(halo)alkyl-O—C₁-C₆-(halo)alkyl, —C₁-C₆-(halo)alkyl-OC(═O)R^{b}, —C₁-C₆-(halo)alkyl-N(R^{b})₂, —C₁-C₆-(halo)alkyl-NR^{b}C(═O)OR^{b}, —C₁-C₆-(halo)alkyl-N(OR^{b})C(═O)OR^{b}, —C₁-C₆-(halo)alkyl-NR^{b}C(═O)N(R^{b})₂, —C₁-C₆-(halo)alkyl-NR^{b}C(═S)N(R^{b})₂, —C₁-C₆-(halo)alkyl-NR^{b}C(═O)R^{b}, —C₁-C₆-(halo)alkyl-NR^{b}C(═S)R^{b}, —C₁-C₆-(halo)alkyl-NR^{b}C(═S)N(R^{b})₂, —C₁-C₆-(halo)alkyl-NR^{b}C(═NR^{b})R^{b}, —C₁-C₆-(halo)alkyl-NR^{b}S(═O)₂R^{b}, —C₁-C₆-(halo)alkyl-N(OR^{b})C(═O)R^{b}, —C₁-C₆-(halo)alkyl-carbocyclyl, —C₁-C₆-(halo)alkyl-aryl, —C₁-C₆-(halo)alkyl-heterocyclyl, —C₁-C₆-(halo)alkyl-heteroaryl, —C₁-C₆-(halo)alkyl-O-carbocyclyl, —C₁-C₆-(halo)alkyl-O-aryl, —C₁-C₆-(halo)alkyl-O-heterocyclyl, —C₁-C₆-(halo)alkyl-O-heteroaryl, —C₁-C₆-(halo)alkyl-C(═O)R^{b}, —C₁-C₆-(halo)alkyl-C(═O)OR^{b}, —C₁-C₆-(halo)alkyl-C(═O)N(R^{b})₂, —C₁-C₆-(halo)alkyl-C(═O)N(OR^{b})R^{b}, —C₁-C₆-(halo)alkyl-C(═S)N(R^{b})₂, —C₁-C₆-(halo)alkyl-OC(═O)N(R^{b})₂, —C₁-C₆-(halo)alkyl-S—R^{b}, —C₁-C₆-(halo)alkyl-S(═O)₂R^{b}, —C₁-C₆-(halo)alkyl-S(═O)₂N(R^{b})₂, —C₁-C₆-(halo)alkyl-C(═NR^{b})R^{b} and —C₁-C₆-alkyl-R^{c} wherein the C₁-C₆-alkyl in said C₁-C₆-alkyl-R^{c} is substituted with two substituents on a same carbon atom that form together with the carbon atom to which they are attached a carbocyclyl,
wherein any carbocyclyl, heterocyclyl, aryl and heteroaryl group may be substituted with one or more R³¹ substituents that may be the same or different,
wherein R³¹ is independently selected from the group consisting of halogen, hydroxy, oxo, cyano, mercapto, sulfinyl, sulfonyl, nitro, C₁-C₆-alkyl, C₁-C₆-haloalkyl, C₁-C₆-hydroxyalkyl, C₁-C₆-cyanoalkyl, C₂-C₆-alkenyl, C₂-C₆-haloalkenyl, C₂-C₆-hydroxyalkenyl, C₂-C₆-cyanoalkenyl, C₂-C₆-alkynyl, C₂-C₆-haloalkynyl, C₂-C₆-hydroxyalkynyl, C₂-C₆-cyanoalkynyl, C₁-C₆-alkoxy, C₁-C₆-haloalkoxy, C₁-C₆-alkylsulfanyl, arylsulfanyl, C₁-C₆-alkylsulfinyl, arylsulfinyl, C₁-C₆-alkylsulfonyl, arylsulfonyl, amino, carbocyclyl, heterocyclyl, aryl, heteroaryl, —O-carbocyclyl, —O-heterocyclyl, —O-aryl, —O-heteroaryl, ═N(OR³¹¹), —N(OR³¹¹)C(═O)OR³¹¹, —N(OR³¹¹)C(═O)R³¹¹, —SO₂N(R³¹¹)₂, —OC(═O)R³¹¹, —N(R³¹¹)₂, —NR³¹¹C(═O)OR³¹¹, —NR³¹¹C(═O)N(R³¹¹)₂, —NR³¹¹C(═O)R³¹¹, —NR³¹¹C(═S)R³¹¹, —NR³¹¹C(═S)N(R³¹¹)₂, —NR³¹¹C(═NR³¹¹)R³¹¹, —N═C—N(R³¹¹)₂, —NR³¹¹S(═O)₂R³¹¹, —OC(═O)N(R³¹¹)₂, —C(═NR³¹¹)R³¹¹, —C(═NOH)R³¹¹, —C(═O)R³¹¹, —C(═O)(OR³¹¹), —C(═O)N(R³¹¹)₂, —C(═O)NR³¹¹N(R³¹¹)₂, —C(═O)N(OR³¹¹)R³¹¹, —C(═S)N(R³¹¹)₂, —C₁-C₆-alkyl-OR³¹¹, —C₁-C₆-alkyl-N(R³¹¹)₂, —C₁-C₆-alkyl-NR³¹¹C(═O)OR³¹¹, —C₁-C₆-alkyl-NR³¹¹C(═O)N(R³¹¹)₂, —C₁-C₆-alkyl-NR³¹¹C(═S)N(R³¹¹)₂, —C₁-C₆-alkyl-NR³¹¹C(═O)R³¹¹, —C₁-C₆-alkyl-NR³¹¹C(═S)R³¹¹, —C₁-C₆-alkyl-NR³¹¹C(═S)N(R³¹¹)₂, —C₁-C₆-alkyl-NR³¹¹C(═NR³¹¹)R³¹¹, —C₁-C₆-alkyl-NR³¹¹S(═O)₂R³¹¹, —C₁-C₆-alkyl-N(OR³¹¹)C(═O)R³¹¹, —C₁-C₆-alkyl-carbocyclyl, —C₁-C₆-alkyl-aryl, —C₁-C₆-alkyl-heterocyclyl, —C₁-C₆-alkyl-heteroaryl, —C₁-C₆-alkyl-C(═O)R³¹¹, —C₁-C₆-alkyl-C(═O)OR³¹¹, —C₁-C₆-alkyl-C(═O)N(R³¹¹)₂, —C₁-C₆-alkyl-C(═O)N(OR³¹¹)R³¹¹, —C₁-C₆-alkyl-C(═S)N(R³¹¹)₂, —C₁-C₆-alkyl-OC(═O)N(R³¹¹)₂, —C₁-C₆-alkyl-S(═O)₂R³¹¹, —C₁-C₆-alkyl-S(═O)₂N(R³¹¹)₂ and —C₁-C₆-alkyl-C(═NR³¹¹)R³¹¹, wherein R³¹¹ is independently hydrogen or C₁-C₆-alkyl,
wherein R^{c} is selected from the group consisting of —OR^{b}, —OC(═O)R^{b}, —N(R^{b})₂, —NR^{b}C(═O)OR^{b}, —N(OR^{b})C(═O)OR^{b}, —NR^{b}C(═O)N(R^{b})₂, —NR^{b}C(═S)N(R^{b})₂, —NR^{b}C(═O)R^{b}, —NR^{b}C(═S)R^{b}, —NR^{b}C(═S)N(R^{b})₂, —NR^{b}C(═NR^{b})R^{b}, —NR^{b}S(═O)₂R^{b}, —N(OR^{b})C(═O)R^{b}, -carbocyclyl, -aryl, -heterocyclyl, -heteroaryl, —O-carbocyclyl, —O-aryl, —O-heterocyclyl, —O— heteroaryl, —C(═O)R^{b}, —C(═O)OR^{b}, —C(═O)N(R^{b})₂, —C(═O)N(OR^{b})R^{b}, —C(═S)N(R^{b})₂, —OC(═O)N(R^{b})₂, —S—R^{b}, —S(═O)₂R^{b}, —S(═O)₂N(R^{b})₂ and —C(═NR^{b})R^{b},
wherein any carbocyclyl, heterocyclyl, aryl and heteroaryl group may be substituted by one or more R^{c1} substituents that may be the same or different,
R^{c1} is independently selected from the group consisting of halogen, hydroxy, oxo, cyano, mercapto, sulfinyl, sulfonyl, nitro, C₁-C₆-alkyl, C₁-C₆-haloalkyl, C₁-C₆-hydroxyalkyl, C₁-C₆-cyanoalkyl, C₂-C₆-alkenyl, C₂-C₆-haloalkenyl, C₂-C₆-hydroxyalkenyl, C₂-C₆-cyanoalkenyl, C₂-C₆-alkynyl, C₂-C₆-haloalkynyl, C₂-C₆-hydroxyalkynyl, C₂-C₆-cyanoalkynyl, C₁-C₆-alkoxy, C₁-C₆-haloalkoxy, C₁-C₆-alkylsulfanyl, arylsulfanyl, C₁-C₆-alkylsulfinyl, arylsulfinyl, C₁-C₆-alkylsulfonyl, arylsulfonyl, amino, carbocyclyl, heterocyclyl, aryl, heteroaryl, —O-carbocyclyl, —O-heterocyclyl, —O-aryl, —O-heteroaryl, ═N(OR^{c11}), —N(OR^{c11})C(═O)OR^{c11}, —N(OR^{c11})C(═O)R^{c11}, —SO₂N(R^{c11})₂, —OC(═O)R^{c11}, —N(R^{c11})₂, —NR^{c11}C(═O)OR^{c11}, —NR^{c11}C(═O)N(R^{c11})₂, —NR^{c11}C(═O)R^{c11}, —NR^{c11}C(═S)R^{c11}, —NR^{c11}C(═S)N(R^{c11})₂, —NR^{c11}C(═NR^{c11})R^{c11}, —N═C—N(R^{c11})₂, —NR^{c11}S(═O)₂R^{c11}, —OC(═O)N(R^{c11})₂, —C(═NR^{c11})R^{c11}, —C(═NOH)R^{c11}, —C(═O)R^{c11}, —C(═O)(OR^{c11}), —C(═O)N(R^{c11})₂, —C(═O)NR^{c11}N(R^{c11})₂, —C(═O)N(OR^{c11})R^{c11}, —C(═S)N(R^{c11})₂, —C₁-C₆-alkyl-OR^{c11}, —C₁-C₆-alkyl-N(R^{c11})₂, —C₁-C₆-alkyl-NR¹¹C(═O)OR^{c11}, —C₁-C₆-alkyl-NR^{c11}C(═O)N(R^{c11})₂, —C₁-C₆-alkyl-NR^{c11}C(═S)N(R^{c11})₂, —C₁-C₆-alkyl-NR^{c11}C(═O)R^{c11}, —C₁-C₆-alkyl-NR^{c11}C(═S)R^{c11}, —C₁-C₆-alkyl-NR^{c11}C(═S)N(R^{c11})₂, —C₁-C₆-alkyl-NR^{c11}C(═NR^{c11})R^{c11}, —C₁-C₆-alkyl-NR^{b11}S(═O)₂R^{c11}, —C₁-C₆-alkyl-N(OR^{c11})C(═O)R^{c11}, —C₁-C₆-alkyl-carbocyclyl, —C₁-C₆-alkyl-aryl, —C₁-C₆-alkyl-heterocyclyl, —C₁-C₆-alkyl-heteroaryl, —C₁-C₆-alkyl-C(═O)R^{c11}, —C₁-C₆-alkyl-C(═O)OR^{c11}, —C₁-C₆-alkyl-C(═O)N(R^{c11})₂, —C₁-C₆-alkyl-C(═O)N(OR^{c11})R^{c11}, —C₁-C₆-alkyl-C(═S)N(R^{c11})₂, —C₁-C₆-alkyl-OC(═O)N(R^{c11})₂, —C₁-C₆-alkyl-S(═O)₂R^{c11}, —C₁-C₆-alkyl-S(═O)₂N(R^{c11})₂ and —C₁-C₆-alkyl-C(═NR^{c11})R^{c11}, wherein R^{c11} is independently hydrogen or C₁-C₆-alkyl,
wherein R^{b} is independently selected from the group consisting of hydrogen, halogen, hydroxy, cyano, mercapto, sulfinyl, sulfonyl, nitro, C₁-C₆-alkyl, C₁-C₆-haloalkyl, C₁-C₆-hydroxyalkyl, C₁-C₆-cyanoalkyl, C₂-C₆-alkenyl, C₂-C₆-haloalkenyl, C₂-C₆-hydroxyalkenyl, C₂-C₆-cyanoalkenyl, C₂-C₆-alkynyl, C₂-C₆-haloalkynyl, C₂-C₆-hydroxyalkynyl, C₂-C₆-cyanoalkynyl, C₁-C₆-alkoxy, C₁-C₆-haloalkoxy, C₁-C₆-alkylsulfanyl, arylsulfanyl, C₁-C₆-alkylsulfinyl, arylsulfinyl, C₁-C₆-alkylsulfonyl, arylsulfonyl, amino, C₁-C₆-alkylamino, carbocyclyl, heterocyclyl, aryl, heteroaryl, —O-carbocyclyl, —O— heterocyclyl, —O-aryl, —O-heteroaryl, —C₁-C₆-alkyl-carbocyclyl, —C₁-C₆-alkyl-aryl, —C₁-C₆-alkyl-heterocyclyl, —C₁-C₆-alkyl-heteroaryl, —C₁-C₆-alkyl-O-carbocyclyl, —C₁-C₆-alkyl-O-aryl, —C₁-C₆-alkyl-O-heterocyclyl, —C₁-C₆-alkyl-O-heteroaryl and —C₁-C₆-alkyl-C₁-C₆-alkoxy,
wherein any carbocyclyl, heterocyclyl, aryl and heteroaryl group may be substituted by one or more R^{b1} substituents that may be the same or different,
R^{b1} is independently selected from the group consisting of halogen, hydroxy, oxo, cyano, mercapto, sulfinyl, sulfonyl, nitro, C₁-C₆-alkyl, C₁-C₆-haloalkyl, C₁-C₆-hydroxyalkyl, C₁-C₆-cyanoalkyl, C₂-C₆-alkenyl, C₂-C₆-haloalkenyl, C₂-C₆-hydroxyalkenyl, C₂-C₆-cyanoalkenyl, C₂-C₆-alkynyl, C₂-C₆-haloalkynyl, C₂-C₆-hydroxyalkynyl, C₂-C₆-cyanoalkynyl, C₁-C₆-alkoxy, C₁-C₆-haloalkoxy, C₁-C₆-alkylsulfanyl, arylsulfanyl, C₁-C₆-alkylsulfinyl, arylsulfinyl, C₁-C₆-alkylsulfonyl, arylsulfonyl, amino, carbocyclyl, heterocyclyl, aryl, heteroaryl, —O-carbocyclyl, —O— heterocyclyl, —O-aryl, —O-heteroaryl, ═N(OR^{b11}) —N(OR^{b11})C(═O)OR^{b11}, —N(OR^{b11})C(═O)R^{b11}, —SO₂N(R^{b11})₂, —OC(═O) R^{b11}, —N(R^{b11})₂, —NR^{b11}C(═O)OR^{b11}, —NR^{b11}C(═O)N(R^{b11})₂, —NR^{b11}C(═O)R^{b11}, —NR^{b11}C(═S)R^{b11}, —NR^{b11}C(═S)N(R^{b11})₂, —NR^{b11}C(═NR^{b11})R^{b11}, —N═C—N(R^{b11})₂, —NR^{b11}S(═O)₂R^{b11}, —OC(═O)N(R^{b11})₂, —C(═NR^{b11})R^{b11}, —C(═NOH)R^{b11}, —C(═O)R^{b11}, —C(═O)(OR^{b11}), —C(═O)N(R^{b11})₂, —C(═O)NR^{b11}N(R^{b11})₂, —C(═O)N(OR^{b11})R^{b11}, —C(═S)N(R^{b11})₂, —C₁-C₆-alkyl-OR^{b11}, —C₁-C₆-alkyl-N(R^{b11})₂, —C₁-C₆-alkyl-NR^{b11}C(═O)OR^{b11}, —C₁-C₆-alkyl-NR^{b11}C(═O)N(R^{b11})₂, —C₁-C₆-alkyl-NR^{b11}C(═S)N(R^{b11})₂, —C₁-C₆-alkyl-NR^{b11}C(═O)R^{b11}, —C₁-C₆-alkyl-NR^{b11}C(═S)R^{b11}, —C₁-C₆-alkyl-NR^{b11}C(═S)N(R^{b11})₂, —C₁-C₆-alkyl-NR^{b11}C(═NR^{b11})R^{b11}, —C₁-C₆-alkyl-NR^{b11}S(═O)₂R^{b11}, —C₁-C₆-alkyl-N(OR^{b11})C(═O)R^{b11}, —C₁-C₆-alkyl-carbocyclyl, —C₁-C₆-alkyl-aryl, —C₁-C₆-alkyl-heterocyclyl, —C₁-C₆-alkyl-heteroaryl, —C₁-C₆-alkyl-C(═O)R^{b11}, —C₁-C₆-alkyl-C(═O)OR^{b11}, —C₁-C₆-alkyl-C(═O)N(R^{b11})₂, —C₁-C₆-alkyl-C(═O)N(OR^{b11})R^{b11}, —C₁-C₆-alkyl-C(═S)N(R^{b11})₂, —C₁-C₆-alkyl-OC(═O)N(R^{b11})₂, —C₁-C₆-alkyl-S(═O)₂R^{b11}, —C₁-C₆-alkyl-S(═O)₂N(R^{b11})₂ and —C₁-C₆-alkyl-C(═NR^{b11})R^{b1}, wherein R^{b11} is independently hydrogen or C₁-C₆-alkyl;
provided compound of formula (I) is not
1-{4-[5-methyl-5-(trifluoromethyl)-4,5-dihydro-1,2,4-oxadiazol-3-yl]phenyl}methanamine;
di-tert-butyl{4-[5-methyl-5-(trifluoromethyl)-4,5-dihydro-1,2,4-oxadiazol-3-yl]benzyl}-2-imidodicarbonate;
1-{4-[5,5-bis(trifluoromethyl)-4,5-dihydro-1,2,4-oxadiazol-3-yl]phenyl}methanamine;
2-{6-[3-amino-5-(trifluoromethyl)phenyl]-3-(isopropylamino)-2-oxopyrazin-1(2H)-yl}-N-{4-[5-methyl-5-(trifluoromethyl)-4,5-dihydro-1,2,4-oxadiazol-3-yl]benzyl}acetamide;
2-{6-[3-amino-5-(trifluoromethyl)phenyl]-3-(isopropylamino)-2-oxopyrazin-1(2H)-yl}-N-{4-[5,5-bis(trifluoromethyl)-4,5-dihydro-1,2,4-oxadiazol-3-yl]benzyl}acetamide;
phenyl[3-phenyl-5,5-bis(trifluoromethyl)-1,2,4-oxadiazol-4(5H)-yl]methanone; and
3-phenyl-5,5-bis(trifluoromethyl)-4,5-dihydro-1,2,4-oxadiazole.

2. The compound according to claim 1 wherein A is selected from the group consisting of 9- or 10-membered bicyclic heterocyclyl, C₆-C₁₀-aryl, 5- or 6-membered monocyclic heteroaryl and 9- or 10-membered bicyclic heteroaryl.

3. The compound according to claim 1 wherein n is different from 0 and R3 is selected from the group consisting of hydroxy, halogen, oxo, C₁-C₆-alkyl, C₁-C₆-alkylsulfanyl, arylsulfanyl, arylsulfinyl, C₁-C₆-alkylsulfonyl, arylsulfonyl, carbocyclyl, heterocyclyl, aryl, heteroaryl, —NR^{b}C(═O)R^{b}, —OC(═O)R^{b}, —N(R^{b})₂, —NR^{b}C(═O)OR^{b}, —N═CR^{b}—N(R^{b})₂, —OC(═O)N(R^{b})₂, —C(═NOH)R^{b}, —C(═NOH)N(R^{b})₂, —C(═O)R^{b}, —C(═O)(OR^{b}), —C(═O)N(R^{b})₂, —C(═O)NR^{b}N(R^{b})₂, —C(═S)N(R^{b})₂, —C₁-C₆-(halo)alkyl-O—C₁-C₆-(halo)alkyl, —C₁-C₆-(halo)alkyl-NR^{b}C(═O)OR^{b}, —C₁-C₆-(halo)alkyl-N(OR^{b})C(═O)OR^{b}, —C₁-C₆-(halo)alkyl-NR^{b}C(═O)R^{b}, —C₁-C₆-(halo)alkyl-N(OR^{b})C(═O)R^{b}, —C₁-C₆-(halo)alkyl-aryl, —C₁-C₆-(halo)alkyl-C(═O)N(R^{b})₂ and —C₁-C₆-alkyl-R^{c} wherein the C₁-C₆-alkyl in said C₁-C₆-alkyl-R^{c} is substituted with two substituents on a same carbon atom that form together with the carbon atom to which they are attached a carbocyclyl, wherein any carbocyclyl, heterocyclyl, aryl and heteroaryl groups may be substituted with one or more R³¹ substituents, as recited in claim 1 and wherein R^{b} is as recited in claim 1.

4. The compound according to claim 4 wherein R^{b} is selected from the group consisting of hydrogen, C₁-C₆-alkyl, C₂-C₆-alkynyl, C₁-C₆-alkylsulfanyl, carbocyclyl, aryl, —C₁-C₆-alkyl-carbocyclyl, —C₁-C₆-alkyl-O-aryl and —C₁-C₆-alkyl-C₁-C₆-alkoxy, wherein any carbocyclyl or aryl, group may be substituted by one or more R^{b1} substituents.

5. The compound according to claim 1 wherein R1 is selected from the group consisting of hydrogen, C₁-C₆-alkyl and —C(═O)R^{a}.

6. The compound according to claim 1 wherein R2 is hydrogen or C₁-C₆-alkyl.

7. The compound according to claim 1 wherein n is 0, 1 or 2.

8. A composition comprising a compound as recited in claim 1 and one or more agriculturally acceptable carriers.

9. A method for controlling one or more unwanted phytopathogenic microorganisms which comprises applying one or more compounds according to claim 1 or a composition thereof plants, plant parts, seeds, fruits or to soil in which the plants grow.

10. A process for preparing a compound according claim 1 which comprises reacting one or more chloroxymes of formula (II) with one or more imines of formula (III) in a solvent

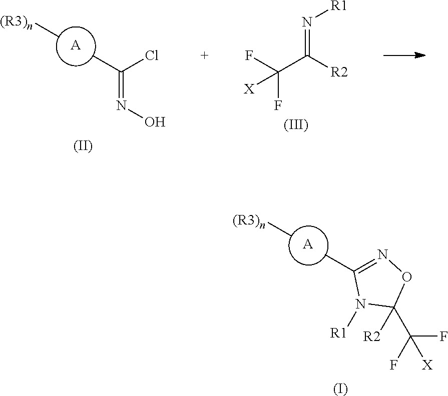

11. A process for preparing a compound as recited in claim 1 wherein R1 and R2 are hydrogen atom (compound of formula I-a) which comprises reacting one or more oxadiazoles of formula (IV) with a reducing agent in a solvent [chemical structure]
